(19) United States
(12) Patent Application Publication
(10) Pub. No.: US 2016/0279175 A1
(43) Pub. Date: Sep. 29, 2016

(54) METHODS FOR REDUCING DEVELOPMENT OF RESISTANCE TO ANTIBIOTICS

(71) Applicant: REGENTS OF THE UNIVERSITY OF MINNESOTA, Minneapolis, MN (US)

(72) Inventors: Yiannis John Kaznessis, New Brighton, MN (US); Brittany Anne Forkus, Minneapolis, MN (US); Kathryn Gayle Geldart, St. Paul, MN (US)

(21) Appl. No.: 15/079,520

(22) Filed: Mar. 24, 2016

Related U.S. Application Data
(60) Provisional application No. 62/138,249, filed on Mar. 25, 2015.

(51) Int. Cl. A61K 35/74; C07K 14/315; A01N 63/00; A61K 31/395

(52) U.S. Cl. CPC A61K 35/74; A61K 31/395; C07K 14/315; A01N 63/00; A61K 2035/11

(57) ABSTRACT

Provided herein are vectors having a polynucleotide encoding a polycistronic mRNA operably linked to a heterologous promoter, where the polycistronic mRNA includes at least two coding regions encoding a first and a second antimicrobial peptide. In one embodiment, the heterologous promoter is controlled by a modulatory protein, such as a GadR, PROTEON, or PROTEOFF modulatory protein. Also provided is a genetically modified microbe that includes a vector described herein, and methods of using the genetically modified microbe. The methods include increasing activity of antimicrobial peptides against a microbial pathogen, reducing development of resistance of a microbial pathogen, and treating a subject having a pathogenic microbe.

### CROSS-REFERENCE TO RELATED APPLICATIONS

This application claims the benefit of U.S. Provisional Application Ser. No. 62/138,249, filed Mar. 25, 2015, which is incorporated by reference herein.

### GOVERNMENT FUNDING

This invention was made with government support under GM111358, awarded by the NIH, and CBET-1412283, awarded by the NSF. The government has certain rights in the invention.

### SEQUENCE LISTING

This application contains a Sequence Listing electronically submitted to the United States Patent and Trademark Office via EFS-Web as an ASCII text file entitled “11004810101_SequenceListing_ST25.txt” having a size of 72 KB and created on Mar. 24, 2016. Due to the electronic filing of the Sequence Listing, the electronically submitted Sequence Listing serves as both the paper copy required by 37 CFR §1.821(c) and the CRF required by §1.821(e). The information contained in the Sequence Listing is incorporated by reference herein.

### SUMMARY OF THE APPLICATION

Provided herein are vectors. In one embodiment, a vector includes a polynucleotide that encodes a polycistronic mRNA operably linked to a heterologous promoter, wherein the polycistronic mRNA includes at least two coding regions encoding a first and a second antimicrobial peptide. In one embodiment, the polycistronic mRNA includes at least three coding regions encoding a first, a second, and a third antimicrobial peptide. In one embodiment, the heterologous promoter is a first promoter, and expression of the operably linked polynucleotide by the first promoter is controlled by a modulatory protein, wherein the vector further includes a second promoter operably linked to an additional coding region that encodes the modulator protein. In one embodiment, the first promoter is a chloride-inducible promoter, and the modulator protein includes a GadR protein. In one embodiment, the heterologous promoter is regulated by a PROTEON or a PROTEOFF modulatory protein. In one embodiment, the polycistronic mRNA is at least 3,000 nucleotides in length.

In one embodiment, the vector further includes a cvaA coding region and a cvaB coding region, and wherein each antimicrobial peptide includes a leader sequence that is recognized by the CvaA and CvaB proteins and is exported from an E. coli cell when the vector is present in the E. coli cell.

Also provided is a genetically modified microbe that includes a vector described herein. In one embodiment, the vector is not integrated into the genomic DNA of the genetically modified microbe. The genetically modified microbe can be a Gram positive microbe, including a lactic acid bacterium such as a Lactococcus spp. or a Lactobacillus spp. The genetically modified microbe can be a Gram negative microbe, such as E. coli.

Also provided are methods. In one embodiment, a method is for increasing activity against a microbial pathogen, and includes exposing a pathogenic microbe to a genetically modified microbe described herein. Expression of the antimicrobial peptides by the genetically modified microbe results in an increase in the amount of time for regrowth of the pathogenic microbe compared to the amount of time for regrowth of the pathogenic microbe when exposed to a genetically modified microbe expressing a single antimicrobial peptide.

In one embodiment, a method is for reducing development of resistance, and includes exposing a pathogenic microbe to a genetically modified microbe described herein. Expression of the antimicrobial peptides by the genetically modified microbe results in a decrease of the fraction of the population of the pathogenic microbe with resistance to an administered antimicrobial peptide compared to the fraction of the population of the pathogenic microbe with resistance to an administered antimicrobial peptide when exposed to a genetically modified microbe expressing a single antimicrobial peptide.

The methods may occur in vitro or in vivo. The pathogenic microbe can be a Gram positive microbe or a Gram negative microbe.

In one embodiment, a method is for treating a subject having a pathogenic microbe, and includes administering to the subject having a pathogenic microbe infection a genetically modified microbe described herein. In one embodiment, the pathogenic microbe is a member of the genus Enterococcus, wherein the antimicrobial peptides include Enterocin A, Enterocin B, Enterocin P, and Hiracin JM79, and the method further includes administering a rifamycin to the subject. In one embodiment, the pathogenic microbe is a member of the genus Salmonella, wherein the antimicrobial peptides includes Microcin V, Microcin 24, and Microcin 25. In one embodiment, the pathogenic microbe is a member of the genus Clostridia, wherein the antimicrobial peptides are selected from Endolysin 170 (Lys170), PlyV12, EFAL-1, ORF9, and Lys168.

As used herein, the term “protein” refers broadly to a polymer of two or more amino acids joined together by peptide bonds. The term “protein” also includes molecules which contain more than one protein joined by a disulfide bond, or complexes of proteins that are joined together, covalently or noncovalently, as multimers (e.g., dimers, trimers, tetramers). Thus, the terms peptide, oligopeptide, enzyme, subunit, and protein are all included within the definition of protein and these terms are used interchangeably. It should be understood that these terms do not connote a specific length of a polymer of amino acids, nor are they intended to imply or distinguish whether the protein is produced using recombinant techniques, chemical or enzymatic synthesis, or is naturally occurring.

As used herein, the term “polynucleotide” refers to a polymeric form of nucleotides of any length, either ribonucleotides or deoxynucleotides, and includes both double- and single-stranded RNA and DNA. A polynucleotide can be obtained directly from a natural source, or can be prepared with the aid of recombinant, enzymatic, or chemical techniques. A polynucleotide can be linear or circular in topology. A polynucleotide may be, for example, a portion of a vector, such as an expression or cloning vector, or a fragment. A polynucleotide may include nucleotide sequences having different functions, including, for instance, coding regions, and non-coding regions such as regulatory regions.

As used herein, the terms “coding region,” “coding sequence,” and “open reading frame” are used interchangeably and refer to a nucleotide sequence that encodes a protein and, when placed under the control of appropriate regulatory sequences expresses the encoded protein. The boundaries of a coding region are generally determined by a translation start codon at its 5′ end and a translation stop codon at its 3′ end. A “regulatory sequence” is a nucleotide sequence that regulates expression of a coding sequence to which it is operably linked. Non-limiting examples of regulatory sequences include promoters, enhancers, transcription initiation sites, translation start sites, translation stop sites, and transcription terminators. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

As used herein, a “polycistronic mRNA” refers to a transcription product that includes two or more coding regions. Expression of the two or more coding regions is controlled by a single promoter, and the series of the two or more coding regions that are transcribed to produce a polycistronic mRNA is referred to as an operon.

As used herein, “genetically modified microbe” refers to a microbe which has been altered “by the hand of man.” A genetically modified microbe includes a microbe into which has been introduced an exogenous polynucleotide, e.g., an expression vector.

As used herein, a “vector” is a replicating polynucleotide, such as a plasmid, to which another polynucleotide may be attached so as to bring about the replication of the attached polynucleotide.

As used herein, an “exogenous protein” and “exogenous polynucleotide” refers to a protein and polynucleotide, respectively, which is not normally or naturally found in a microbe, and/or has been introduced into a microbe. An exogenous polynucleotide may be separate from the genomic DNA of a cell (e.g., it may be a vector, such as a plasmid), or an exogenous polynucleotide may be integrated into the genomic DNA of a cell.

As used herein, a “heterologous” polynucleotide, such as a heterologous promoter, refers to a polynucleotide that is not normally or naturally found in nature operably linked to another polynucleotide, such as a coding region. As used herein, a “heterologous” protein or “heterologous” amino acids refers to amino acids that are not normally or naturally found in nature flanking an amino acid sequence.

As used herein, a protein may be “structurally similar” to a reference protein if the amino acid sequence of the protein possesses a specified amount of sequence similarity and/or sequence identity compared to the reference protein. Thus, a protein may be “structurally similar” to a reference protein if, compared to the reference protein, it possesses a sufficient level of amino acid sequence identity, amino acid sequence similarity, or a combination thereof.

Structural similarity of two proteins can be determined by aligning the residues of the two proteins (for example, a candidate protein and any appropriate reference protein described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A reference protein may be a protein described herein. A candidate protein is the protein being compared to the reference protein. A candidate protein may be isolated, for example, from a microbe, or can be produced using recombinant techniques, or chemically or enzymatically synthesized.

Unless modified as otherwise described herein, a pair-wise comparison analysis of amino acid sequences can be carried out using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on. Alternatively, polypeptides may be compared using the BESTFIT algorithm in the GCG package (version 10.2, Madison Wis.).

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions.

Thus, as used herein, a candidate protein useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to a reference amino acid sequence.

Alternatively, as used herein, a candidate protein useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference amino acid sequence.

Conditions that are “suitable” for an event to occur, such as expression of an exogenous polynucleotide in a cell to produce a protein, or production of a product, or “suitable” conditions are conditions that do not prevent such events from occurring. Thus, these conditions permit, enhance, facilitate, and/or are conducive to the event.

As used herein, an “animal” includes members of the class Mammalia and members of the class Ayes, such as human, avian, bovine, caprine, ovine, porcine, equine, canine, and feline.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of and/or “consisting essentially of are also provided.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Maps of pNZC and inserts used in this study. Chloride-inducible promoter (CIP) was inserted between BglII and NcoI. PgadR is a constitutive promoter controlling the production of the activator protein GadR. LacZ and AMP expression are controlled by chloride-inducible promoter Pgad (activated by GadR). LacZ and Bac are inserted between cut sites NcoI and SpeI in pNZC.

FIG. 2. Comparison of β-galactosidase production under the nisin-inducible promoter and the chloride-inducible promoter in L. lactis NZ9000. The nisin-inducible promoter was induced with 0 ng/mL, 5 ng/mL, or 40 ng/mL nisin and the chloride-inducible promoter was induced with 0.01 M, 0.05 M, 0.1 M, 0.3 M, and 0.5 M Cl—. Cells were induced at an OD600 of ˜0.45 then sampled at the designated time post-induction. Error bars represent +/− one standard deviation of assay triplicates.

FIG. 3. Agar diffusion inhibition test of L. lactis NZ9000 producing Enterocin A, Enterocin P, and Hiracin JM79 under the CIP (pNZCA3) or with no AMPs under the CIP (pNZC) at different chloride concentrations. E. faecium 8-E9 was used as the indicator strain. Modified GM17 medium was used to obtain a lower basal level chloride concentration (0.005 M).

FIG. 4. L. lactis pNZCA3 growth in modified GM17 medium with 0.005 M, 0.05 M, 0.125 M, 0.3 M, and 0.5 M NaCl. Error bars represent +/− one standard deviation calculated from biological triplicates.

FIG. 5. E. faecium 8-E9 growth in modified GM17 medium with 0.005 M, 0.05 M, 0.125 M, 0.3 M, and 0.5 M NaCl. Error bars represent +/− one standard deviation calculated from biological triplicates.

FIG. 6. Agar diffusion inhibition test of L. lactis NZ9000 producing Enterocin A, Enterocin P, and Hiracin JM79 under the CIP (pNZCA3) against 10 strains of Enterococcus. Indicator strain is shown below each halo. See Methods section for descriptions of strains. Halo diameters range from ˜8-13 mm in diameter. Tests were done in BHI+agar (˜0.15 M chloride).

FIG. 7a. E. faecium growth when grown alone (red) or with (teal) L. lactis producing Enterocin A, Enterocin P, and Hiracin JM79 under the chloride-inducible promoter at 0.01 M, 0.1 M, and 0.3 M chloride. Data points represent the averages of three technical replicates. Error bars represent +/− one standard deviation calculated from the sample triplicates.

FIG. 7b. L. lactis pNZCA3 counts from the co-culture test in FIG. 5a. Note the reduced growth at higher salt concentrations. Data points represent the averages of three technical replicates. Error bars represent +/− one standard deviation calculated from the sample triplicates.

FIG. 8. Stability of E. faecium resistance to AMPS after 10 generations of re-growth. Blue curve shows wild type E. faecium growth in the absence of AMPs and red curve shows wild type E. faecium growth in the presence of AMPs. After 15 hours, E. faecium grown in the presence of AMPS (red) were inoculated into GM17 with no AMPs and grown for 10 generations. The green curve shows the growth of the resulting E. faecium grown in the absence of AMPs and the purple curve shows the growth of this same E. faecium grown in the presence of AMPs. Results indicate resistance is maintained for at least 10 generations. Error bars represent +/− one standard deviation of biological triplicates.

FIG. 9. E. faecium inhibition by the addition of 30 μg/mL rifampicin, supernatant from L. lactis producing Enterocin A, Enterocin P, Hiracin JM79 under the CIP (L. lactis pNZCA3), supernatant and rifampicin, or culture containing L. lactis pNZCA3 induced with 0.15 M chloride. Data points represent the averages of three technical replicates. Error bars represent +/− one standard deviation calculated from the sample triplicates.

FIG. 10. Timeline of animal model experiment.

FIG. 11. CFU/g feces of Enterococcus faecium JL282. Results are shown for 6 time points. Fecal samples were collected and plated on Enterococcus faecium JL282 selective media.

FIG. 12. Left panel: Average and standard error of CFU/g feces of Enterococcus faecium JL282 in two groups of mice; treated with sterile water (JL282) and treated with L. lactis NZ9000+PNZCA3 (JL282+Lactis). Right panel: CFU/g of Enterococcus faecium JL282 in the two groups of mice on day 34.

FIG. 13. Combination treatments with enterocin B. E. faecium 8E9 growth in medium containing 1 μg/mL purified enterocin B, 10% supernatant from L. lactis pNZCA3 culture (SN), 10% supernatant and 1 μg/mL purified enterocin B, or 10% supernatant and 10 μg/mL purified enterocin B. No treatment was added to the control culture.

FIG. 14. Enterocin B is active against class IIa resistant mutants. Halo test of L. lactis pNZCA3, 1 μg enterocin B, and 0.1 μg enterocin B. E. faecium 6E6 wild type and class IIa resistant mutant were used as indicator strains.

FIG. 15. Production and secretion of Enterocin B from L. lactis. From the left, halo test of L. lactis producing Enterocin B with Divergicin A secretion peptide, Enterocin B with usp45 secretion peptide, and Enterocin A with the usp45 secretion peptide. E. faecium 8E9 was used as the indicator strain.

FIG. 16. Vector map of pMPES. HindIII-SalI contains the 9.4 kb region encoding all necessary MicV secretion machinery (cvaA and cvaB). A point mutation was inserted in the MicV structural gene, cvac, to eliminate native AMP expression. Cm encodes the chloramphenicol resistance gene and cvi encodes the native MicV immunity protein. The multiple cloning site (MCS) is shown in detail in FIG. 18.

FIG. 17. Production and secretion of Microcin V, Microcin L, and Microcin N, and Enterocin A from pMPES machinery. White dots are E. coli MC1061 F′ transformed with pHK22ΔPP expressing AMPs with the native signal peptide or the AMP with the MicV signal peptide (Vsp). E. coli DH5α was used as the indicator strain in the left panel of FIG. 16 for MicV, MicL, and MicN and E. faecium 8E9 was used as the indicator in the right panel of FIG. 16 for EntA. Note, pHK22ΔPP is operationally identical to pMPES, and pNZCA encodies EntA under control of the chloride promoter.

### DETAILED DESCRIPTION OF ILLUSTRATIVE EMBODIMENTS

The inventors have observed in recent studies that cocktails of antimicrobial peptides (AMPs) result in stronger inhibition and higher reduction of pathogenic cell counts. Peptides such as PG-1, Microcin J25 and colicin have varying mechanisms of action, and a combination can act synergistically against microbes. Initial results include L. lactis producing enterocins that target Enterococcus faecium, and establishing that three peptides, Enterocin A, Enterocin P and Hiracin JM79, expressed from a single poly-cistronic DNA construct, inhibit enterococcus more effectively than each of the peptides acting alone.

Interestingly, a combination of the three peptides with traditional antibiotics resulted in remarkable synergies. In particular, the combination of rifampicin with the peptides drastically reduced E. faecium and stopped the regrowth of the surviving E. faecium. Other antibiotics, including ampicillin, erythromycin, and chloramphenicol did not exhibit similarly impressive reductions in counts of E. faecium.

Provided herein are vectors and genetically modified microbes that include one or more of the vectors. In one embodiment, a vector includes a transcription unit that encodes a polycistronic mRNA and an operably linked heterologous promoter. The transcription unit and resulting polycistronic mRNA includes at least two, at least three, or at least four coding regions, where each coding region encodes a different antimicrobial peptide. In one embodiment, the polycistronic mRNA includes a first coding region encoding a first antimicrobial protein, a second coding region encoding a second antimicrobial protein, and a third coding region encoding a third antimicrobial protein. In one embodiment, the polycistronic mRNA includes additional coding regions encoding additional antimicrobial proteins, for instance, a fourth, a fifth, or a sixth antimicrobial protein. The use of multiple antimicrobial peptides is expected to decrease target microbes in environments such as an animal's gastrointestinal tract, and to reduce the likelihood a target microbe will develop resistance and circumvent therapy.

The heterologous promoter is a controllable promoter. The vector may optionally include another coding region that is operably linked to a second promoter, where the coding region encodes a protein (e.g., a modulatory protein) that regulates the expression of the first promoter. Thus, in one embodiment the vectors described herein are a system for controlling expression of antimicrobial peptides. In one embodiment described herein, a genetically modified microbe expresses antimicrobial peptides only when it has sensed the presence of a specific environment.

Antimicrobial peptides are small proteins, typically between 10 and 100 amino acids in length that inhibit, and often kill, certain bacteria. In one embodiment, an antimicrobial peptide has antimicrobial activity for Gram negative microbes that is greater than the antimicrobial activity it has against Gram positive microbes. In one embodiment, an antimicrobial peptide has essentially similar antimicrobial activity for Gram negative microbes and Gram positive microbes. In one embodiment, an antimicrobial peptide has antimicrobial activity for Gram positive microbes that is greater than the antimicrobial activity it has against Gram negative microbes.

An antimicrobial peptide has antimicrobial activity that inhibits or kills a target microbe. The target microbe may be a Gram negative such as E. coli or a member of the genus Salmonella. Examples of Salmonella include, for instance, Salmonella enterica serotypes Typhimurium, Enteritidis, Gallinarum, Pullorum, Saintpaul, Kentuky, Indiana, Hadar and Heidelberg. Examples of E. coli include, for instance, strains O157:H7, O104:H4, O121, O26, O103, O111, O145, and O104:H21. The target microbe may be a Gram positive such as a member of the genus Enterococcus. Examples of Enterococcus spp. include, for instance, E. faecalis and E. faecium. The target microbe may be in vitro or in vivo. For instance, in one embodiment, a target microbe may be one that is present in the gastrointestinal tract or urogenital system of a subject, and optionally may be pathogenic to the subject. For instance, in another embodiment, a target microbe may be one that is present in the ovaries of hens, contaminating the eggs inside the chicken before the shells are formed.

Whether an antimicrobial peptide has antimicrobial activity can be determined using different indicator strains. Examples of indicator strains include, but are not limited to, pathogenic Salmonella, enterohemorrhagic E. coli O157:H7, lactic acid bacteria such as Lactococcus lactis, Lactobacillus acidophilus, Lb. reuteri, Lb. sakei and Lb. bulgaricus, and Enterococcus spp. Examples of suitable indicator strains include, but are not limited to, those listed in Table 1. In one embodiment, an indicator strain is a member of the genus Enterococcus, such as E. faecalis and E. faecium. Methods for testing the activity of an antimicrobial peptide include, but are not limited to, the stab-on-agar test as well as other methods useful for evaluating the activity of bacteriocins. Such methods are known in the art and are routine.

TABLE 1
Examples of indicator strains.

```
Escherichia coli serotype O157:H7
Salmonella enterica subsp enterica
  serovar Typhimurium
  serovar Tennessee
  serovar St. Paul
  serovar Infantis
Lactococcus lactis subsp lactis IL1403
Lactobacillus acidophilus ATCC 4356
Lactobacillus bulgaricus ATCC 11842
Enterococcus faecalis ATCC 700802
Enterococcus faecalis ATCC 47077
```

An antimicrobial peptide may be naturally occurring or may be engineered. Antimicrobial peptides are produced by all classes of organisms, including mammals, bacteria, and phage. Examples of antimicrobial peptides are shown in Table 2. Examples of antimicrobial peptides from bacteria are bacteriocins, and include class I and class II bacteriocins. An example of class II bacteriocins includes members of the subclass IIa bacteriocins. Class IIa bacteriocins are small (usually 37 to 48 amino acid), heat-stable, and non-post-translationally modified proteins that are typically positively charged and may contain an N-terminal consensus sequence -Tyr-Gly-Asn-Gly-(Val/Lys)-Xaa-Cys-. Examples of class IIa bacteriocins include, but are not limited to, those described in Table 2. Another example of class II bacteriocins includes members of the subclass IIb bacteriocins. Class IIb bacteriocins are heterodimeric bacteriocins that require two different molecules at approximately equal concentrations to exhibit optimal activity.

An interesting structural characteristic of all class IIb bacteriocins characterized so far is that they all contain GxxxG and GxxxG-like motifs. These are generally prevalent in transmembrane peptides and membrane proteins. It has been suggested that these motifs facilitate helix—helix interactions and promote the oligomerization of transmembrane helical peptides or domains of membrane proteins. The hypothesis is that two glycine residues, when separated by three other amino acids, lie at the same face of the helix. Due to their small size, they create a relatively flat surface that may mediate more pronounced helix—helix interactions. Examples of class IIb bacteriocins include, but are not limited, to those described in Table 2. Another example of antimicrobial peptides includes endolysins. Endolysins are double-stranded DNA bacteriophage-encoded peptidoglycan hydrolases produced in phage-infected bacterial cells, and cause rapid lysis when applied to Gram-positive bacteria (Fenton et al., 2010, Bioeng Bugs. 1:9-16; Fischetti, 2008, Curr Opin Microbiol. 11:393-400). Other antimicrobial peptides are known to the person skilled in the art and may also be used as described herein. The present disclosure is not limited by the antimicrobial peptide.

TABLE 2
Examples of antimicrobial peptides.

| Antimicrobial | | |
|---|---|---|
| Peptide | Amino acid sequence | Origin |
| Enterocin A | TTHSGKYYGNGVYCTKNKCTVDWAKATTCIAGMSIGGFLGGAI | E. faecium (1) |
| (EntA) | PGKC (SEQ ID NO: 11) | |
| Enterocin P | ATRSYGNGVYCNNSKCWVNWGEAKENIAGIVISGWASGLAG | E. faecium (2) |
| (EntP) | MGH (SEQ ID NO: 12) | |
| Enterocin B | ENDHRMPNELNRPNNLSKGGAKCGAAIAGGLFGIPKGPLAW | E. faecium (23) |
| | AAGLANVYSKCN (SEQ ID NO: 7) | |
| Hiracin JM79 | ATYYGNGLYCNKEKCWVDWNQAKGEIGKIIVNGWVNHGPW | E. hirae (3) |
| (HirJM79) | APRR (SEQ ID NO: 13) | |
| Protegrin 1 | RGGRLCYCRRRFCVCVGR (SEQ ID NO: 14) | Pig leukocyte (5) |
| (PG-1) | | |
| PC64A | LTYCRRRFCVTV (SEQ ID NO: 15) | PG-1 analogue (6) |
| A3-APO | RPDKPRPYLPRPRPPRPVR (SEQ ID NO: 16) | Engineered antimicrobial |
| | | peptide (7) |
| Alyteserin-1a | GLKDIFKAGLGSLVKGIAAHVAN (SEQ ID NO: 17) | Peptide from the skin of the |
| | | frog Alytes obstetricans (8) |
| Fowlicidin | RVKRVWPLVIRTVIAGYNLYRAIKKK (SEQ ID NO: 18) | Cathelicidin from chicken (9) |
| Microcin 24 | AGDPLADPNSQIVRQIMSNAAWGPPLVPERFRGMAVGAAG | Escherichia coli (10) |
| | GVTQTVLQGAAAHMPVNVPIPKVPMGPSWNGSKG | |
| | (SEQ ID NO: 19) | |
| Colicin V | ASGRDIAMAIGTLSGQFVAGGIGAAAGGVAGGAIYDYASTHK | Escherichia coli (11) |
| (Microcin V) | PNPAMSPSGLGGTIKQKPEGIPSEAWNYAAGRLCNWSPNNLS | |
| | DVCL (SEQ ID NO: 20) | |
| Acidocin LCHV | NVGVLNPPPLV (SEQ ID NO: 21) | Bacteriocin from Lb. |
| NP | | acidophilus n.v. Er 317/402 |
| | | strain Narine (12) |
| Acidocin LCHV | NVGVLNPPMLV (SEQ ID NO: 22) | Bacteriocin from Lb. |
| HV | | acidophilus n.v. Er 317/402 |
| | | strain Narine (12) |
| Acidocin LCHV | NVGVLLPPPLV (SEQ D NO: 23) | Bacteriocin from Lb. |
| LP | | acidophilus n.v. Er 317/402 |
| | | strain Narine (12) |
| Acidocin LCHV | NVGVLLPPMLV (SEQ ID NO: 24) | Bacteriocin from Lb. |
| LM | | acidophilus n.v. Er 317/402 |
| | | strain Narine (12) |
| LGG NPSRQERR | NPSRQERR (SEQ ID NO: 25) | Small bioactive peptide from |
| | | Lactobacillus GG (12) |
| LGG PDENK | PDENK (SEQ ID NO: 26) | Small bioactive peptide from |
| | | Lactobacillus GG (13) |
| Endolysin 170 | MAGEVFSSLITSVNPNPMNAGSRNGIPIDTIILHHNATTNKDV | E. faecalis phage F170/08 (4) |
| (Lys170) | AMNTWLLGGGAGTSAHYECTPTEIIGCVGEQYSAFHAGGTGG | |
| | IDVPKIANPNQRSIGIENVNSSGAPNWSVDPRTITNCARLVADI | |
| | CTRYGIPCDRQHVLGHNEVTATACPGGMDVDEVVRQAQQF | |
| | MAGGSNNAVKPEPSKPTPSKPSNNKNKEGVATMYCLYERPIN | |
| | SKTGVLEWNGDAWTVMFCNGVNCRRVSHPDEMKVIEDIYRK | |
| | NNGKDIPFYSQKEWNKNAPWYNRLETVCPVVGITKKS | |
| | (SEQ ID NO: 27) | |
| PlyV12 | MSNINMETAIANMYALKARGITYSMNYSRTGADGTGDCSGTV | Encoded by phage F1 (14) |
| | YDSLRKAGASDAGWVLNTDSMHSWLEKNGFKLIAQNKEWSA | |
| | KRGDVVIFGKKGASGGSAGHVVIFISSTQIIHCTWKSATANGVY | |
| | VDNEATTCPYSMGWYVYRLNGGSTPPKPNTKKVKVLKHATN | |
| | WSPSSKGAKMASFVKGGTFEVKQQRPISYSYSNQEYLIVNKGT | |
| | VLGWVLSQDIEGGYGSDRVGGSKPKLPAGFTKEEATFINGNAP | |
| | ITTRKNKPSLSSQTATPLYPGQSVRYLGWKSAEGYIWIYATDGR | |
| | YIPVRPVGKEAWGTFKQDIEGGYGSDRVGGSKPKLPAGFTKEE | |
| | ATFINGNAPITTRKNKPSLSSQTATPLYPGQSVRYLGWKSAEGY | |
| | IWIYATDGRYIPVRPVGKEAWGTFK (SEQ ID NO: 28) | |
| EFAL-1 | MKLKGILLSVVTTFGLLFGATNVQAYEVNNEFNLQPWEGSQQ | Produced by phage EFAP-1 (15) |
| | LAYPNKIILHETANPRATGRNEATYMKNNWFNAHTTAIVGDG | |
| | GIVYKVAPEGNVSWGAGNANPYAPVQIELQHTNDPELFKANY | |
| | KAYVDYTRDMGKKFGIPMTLDQGGSLWEKGVVSHQWVTDF | |
| | VWGDHTDPYGYLAKMGISKAQLAHDLANGVSGNTATPTPKP | |
| | DKPKPTQPSKPSNKKRFNYRVDGLEYVNGMWQIYNEHLGKID | |
| | FNWTENGIPVEVVDKVNPATGQPTKDQVLKVGDYFNFQENST | |
| | GVVQEQTPYMGYTLSHVQLPNEFIWLFTDSKQALMYQ | |
| | (SEQ ID NO: 29) | |
| ORF9 | MAGEVFSSLITSVNPNPMNAGSRNGIPIDTIILHHNATTNKDV | From phage jEF24C (16) |
| | AMNTWLLGGGAGTSAHYECTPTEIIGCVGEQYSAFHAGGTGG | |
| | IDVPKIANPNQRSIGIENVNSSGAPNWSVDPRTITNCARLVADI | |
| | CTRYGIPCDRQHVLGHNEVTATACPGGMDVDEVVRQAQQF | |
| | MAGGSNNAVKPEPSKPTPSKPSNNKNKEGVATMYCLYERPIN | |
| | SKTGVLEWNGDAWTVMFCNGVNCRRVSHPDEMKVIEDIYRK | |
| | NNGKDIPFYSQKEWNKNAPWYNRLETVCPVVGITKKS | |
| | (SEQ ID NO: 30) | |
| Lys168 | MVKLNDVLSYVNGLVGKGVDADGWYGTQCMDLTVDVMQR | From phage F168/08 (17) |
| | FFGWRPYGNAIALVDQPIPAGFQRIRTTSSTQIKAGDVMIWGL | |
| | GYYAQYGHTHIATEDGRADGTFVSVDQNWINPSLEVGSPAAA | |
| | IHHNMDGVWGVIRPPYEAESKPKPPAPKPDKPNLGQFKGDD | |
| | DIMFIYYKKTKQGSTEQWFVIGGKRIYLPTMTYVNEANDLIKRY | |
| | GGNTNVTTYNYDNFGLAMMEKAYPQVKL (SEQ ID NO: 31) | |
| Microcin J25 | cyclo(-G1GAGHVPEYF₁₀VGIGTPISFY₂₀G-) | E. coli (18) |
| Plantaricin JK | PlnJ GAWKNFWSSLRKGFYDGEAGRAIRR | Class IIb heterodimeric |
| (PlnJK). | (SEQ ID NO: 3) | bacteriocin produced by |
| Plantaricin | PlnK RRSRKNGIGYAIGYAFGAVERAVLGGSRDYNK | Lactobacillus plantarum C11 |
| JK is comprised | (SEQ ID NO: 4) | (19) |
| of the two | | |
| peptides | | |
| Plantaricin J | | |
| (PlnJ) | | |
| and Plantaricin K | | |
| (PlnK) | | |
| Plantaricin EF | PlnE FNRGG YNFGKSVRHVVDAIGSVAGIRGILKSIR | Class IIb heterodimeric |
| (PlnEF). | (SEQ ID NO: 5) | bacteriocin produced by |
| Plantaricin | PlnF VFHAYSARGVRNNYKSAVGPADWVISAVRGFIHG | Lactobacillus plantarum C11 |
| EF is comprised | (SEQ ID NO: 6) | (20) |
| of the two | | |
| peptides | | |
| Plantaricin E | | |
| (PlnE) | | |
| and Plantaricin F | | |
| (PlnF) | | |
| Microcin N | AGDPLADPNSQIVRQIMSNAAWGAAFGARGGLGGMAVGAA | Produced by E. coli (21) |
| | GGVTQTVLQGAAAHMPVNVPIPKVPMGPSWNGSKG | |
| | (SEQ ID NO: 8) | |
| Microcin L | GDVNWVDVGKTVATNGAGVIGGAFGAGLCGPVCAGAFAVG | Produced by E. coli (22) |
| | SSAAVAALYDAAGNSNSAKQKPEGLPPEAWNYAEGRMCNW | |
| | SPNNLSDVCL (SEQ ID NO: 9) | |

1. Aymerich et al., 1996, Appl Environ Microbiol. 62:1676-1682; 2. Cintas et al., 1997, Appl Environ Microbiol., 63:4321-4330; 3. Sánchez et al., 2007, FEMS Microbiol Lett. 270:227-236; 4. Proença et al., 2012, Microb Drug Resist., 18:322-332; 5. Fahrner et al., 1996, Chemistry & Biology 3:543-550; 6, Chang et al., U.S. Patent No. 5,994,306; 7. Szabo et al., 2010, International journal of antimicrobial agents 35:357-361; 8. Conlon et al., 2009, Peptides 30, 1069-1073; 9. Xiao, 2005, Journal of Biological Chemistry 281:2858-2867; 10. O'Brien et al., 1994, Plasmid 31:288-296; 11. Gillor et al., 2004, Advances in applied microbiology 54:129-146; 12. Mkrtchyan et al., 2010, International journal of antimicrobial agents 35:255-260; 13. Lu et al., 2009,1 Pediatr. Gastroenterol. Nutr. 49:23-30; 14. Yoong et al., 2004, J. Bacteriol. 186:4808-4812; 15. Uchiyama et al., 2011, Appl Environ Microbiol. 77:580-585; 16. Son et al., 2010, J. Appl Microbiol. 108:1769-1779; 17. Proença et al., 2012, .Microb Drug Resist. 18: 322-332; 18, Salomon and Farias, 1992,1 Bacteriol. 174:7428-7435; 19: Hauge et al. 1999,1 Bacteriol., 181(3):740-7; 20: Kyriakou et al., 2016, Biochim Biophys Acta. 1858(4):824-35; 21: Corsini et al., 2010, FEMS Microbiol Lett. 312(2):119-25.22: Pons et al., 2004, Antimicrob Agents Chemother. 48(2):505-13. 23: Casaus et al., 1997, Microbiology. 143 (Pt 7):2287-94.

Examples of antimicrobial peptides also include those that are essentially identical to any one of the antimicrobial peptides in Table 2. As used herein, in the context of a protein “essentially identical” refers to a protein that differs from one of the proteins disclosed herein. A protein that is essentially identical to an antimicrobial peptide differs from one of the antimicrobial peptides in in Table 2 at 1, 2, 3, 4, 5, 6, 7, 8, 9, 10, 11, or 12 amino acid residues and has antimicrobial activity. In one embodiment, the difference is a conservative substitution. Conservative amino acid substitutions are defined to result from exchange of amino acids residues from within one of the following classes of residues: Class 1: Ala, Gly, Ser, Thr, and Pro (representing small aliphatic side chains and hydroxyl group side chains); Class 2: Cys, Ser, Thr, and Tyr (representing side chains including an —OH or —SH group); Class 3: Glu, Asp, Asn, and Gln (carboxyl group containing side chains): Class 4: His, Arg, and Lys (representing basic side chains); Class 5: Ile, Val, Leu, Phe, and Met (representing hydrophobic side chains); and Class 6: Phe, Trp, Tyr, and His (representing aromatic side chains).

A nucleotide sequence of a coding sequence encoding an antimicrobial peptide may be easily predicted based on reference to the standard genetic code. When an antimicrobial peptide is to be expressed in a particular microbe, a nucleotide sequence encoding the antimicrobial peptide may be produced with reference to preferred codon usage for the particular microbe.

In one embodiment, the antimicrobial peptides encoded by the vector are selected to have different mechanisms of action. The inventors have found that exposing a target microbe to two, three, or more antimicrobial peptides that each have a different mechanism of action results in a greater reduction of the target microbe and a reduction in the target microbe's ability to develop resistance. Examples of different mechanisms of action include, but are not limited to, affecting the structural integrity of bacteria cell membranes, inhibiting activity of DNA-dependent RNA polymerase, and disrupting the integrity of manose phosphotransferase or other membrane proteins that transport molecules. In one embodiment, an antimicrobial peptide may act as an immunomodulator of an animal's immune system. For instance, an antimicrobial peptide may induce animal immune cells to produce cytokines and/or chemokines, and attract and/or activate immune cells. Such immune system activation may aid in the clearance of a pathogenic microbe. Examples of different mechanisms of action and antimicrobial peptides having that mechanism are shown in Table 3 (see also Guilhelmelli et al., 2013, Frontiers in Microbiol., 4: 353, doi: 10.3389/fmicb.2013.00353).

TABLE 3

| Mechanism of action | Examples of antimicrobial peptides |
|---|---|
| Membrane disrupting | Protegrin, Colicins, Microcin V, MccE492 |
| Inhibition of Cell Wall | Lantibiotics (class I AMPs), for example, Nisin |
| Synthesis | |
| Inhibition of Nucleic | Indolicidin, Puroindoline, PR-39, Microcidin |
| Acid Synthesis | B17 |
| Targeting RNA | Microcin J25, Microcin C |
| polymerase | |
| Targeting Mannose | Class IIa bacteriocons, including Enterocin A, |
| phosphotransferase | Enterocin P, Hiracin JM79 |

A coding sequence encoding an antimicrobial peptide may further include nucleotides encoding a secretion signaling protein, such that the antimicrobial peptide and the secretion signaling protein are fused and expressed as a single protein. A secretion signaling protein targets a protein for secretion out of the cell, and is usually present at the amino-terminal end of a protein. Secretion signaling proteins useful in prokaryotic microbes are known in the art and routinely used. Examples of secretion signaling proteins useful in lactic acid bacteria, including L. lactis, Lb. acidophilus, Lb. acidophilus, Lb. bulgaricus, Lb. reuteri, and Lb. plantarum are known. One example of a useful secretion signaling protein is from the protein Usp45 (Van Asseldonk et al., 1990, Gene, 95, 155-160). Several variations on Usp45 have been explored and may also be employed (Ng and Sarkar, 2012, Appl. Environ. Microbiol., 79:347-356). Additionally, lactobacillus secretion tags including but not limited to Lp_3050 and Lp_2145 may be used in L. lactis and Lactobacilli spp.

In addition to the signal peptides mentioned above which rely on the general Sec secretion machinery, many antimicrobial peptides also have their own dedicated secretion machinery with corresponding secretion tags. These tags are typically associated with the antimicrobial peptide natively secreted by these transport systems, however, these tags can also be used to secrete non-native antimicrobial peptides. An example of this mechanism of secretion is a double-glycine-type leader, which has been used to secrete colicin V from L. lactis. In the majority of microcin transport systems, secretion systems are associated with self-immunity or proteolytic cleavage of the microcin precursor. The Class II microcin gene clusters often encode for a dedicated ABC transporter and an accessory protein.

In one embodiment, a coding sequence encoding an antimicrobial peptide may further include nucleotides encoding a cathelin, such that the antimicrobial peptide is not active until the cathelin amino acids are removed. Cathelin is a pro-peptide sequence present on cathelicidin-related antimicrobial peptides that are biologically inactive. Cathelicidin-related antimicrobial peptides are produced in neutrophils and stored in non-active form with the N-terminus cathelin pro-peptide (Kokryakov et al., FEBS Lett, 1993, 327(2):231-236; Qu et al., Infect Immun, 1997, 65(2):636-639; Tamamura et al., Chemical & Pharmaceutical Bulletin, 1995, 43(5): 853-858). These pro-peptides are cleaved proteolytically, rendering the attached cathelicidin-related antimicrobial peptide active. A cathelin pro-peptide can be designed with a sequence for cleavage by trypsin, a ubiquitous gut enzyme. A sequence for cleavage by trypsin may include Arg or Lys, where the cleavage occurs immediately after the Arg Lys. Amino acid sequences that are readily cleaved by trypsin are known to the skilled person and routinely used.

In one embodiment, the polynucleotide that encodes the polycistronic mRNA includes further coding regions in addition to the at least two coding regions encoding antimicrobial peptides. An additional coding region may be useful in processing an antimicrobial peptide, exporting an antimicrobial peptide out of a microbial cell, or making the microbial cell immune to the action of the antimicrobial peptide. Genes encoding antimicrobial peptides are typically located adjacent to other genes encoding these accessory proteins, and these genes, and the proteins they encode, are known to the skilled person and are readily available.

Due to the inclusion of multiple coding regions in a polycistronic mRNA, e.g., coding regions encoding two or three antimicrobial peptides and the coding regions for accessory proteins for each antimicrobial peptide, a polycistronic mRNA may be at least 2,500 nucleotides, at least 3,000 nucleotides, at least 3,500 nucleotides, at least 4,000 nucleotides, at least 4,500 nucleotides, or at least 5,000 nucleotides in length. In those embodiments where a polycistronic mRNA may be at least 2,500 nucleotides long, useful promoters are those strong enough to drive transcription of the entire mRNA, e.g., there is little premature termination of transcription. Minimal premature transcription termination results in the translation of substantially equal amounts of each antimicrobial peptide. In some embodiments, a polycistronic mRNA may be at least 50, at least 100, or at least 150 nucleotides when shorter antimicrobial peptides are encoded.

In one embodiment, an antimicrobial peptide, such as an antimicrobial peptide that does not use the general secretory pathway for export, can be modified to exchange the native secretion tag with a secretion tag from Microcin V. An example of a Microcin V secretion tag is MRTLTLNELDSVSGG (SEQ ID NO:32). This tag is identified, processed, and exported by the product of the cvaA and cvaB coding regions, CvaA and CvaB, respectively. This type of modification can allow an antimicrobial peptide to be exported from the cell by the secretion machinery that secretes Microcin V. In another embodiment, the secretion machinery that secretes the Microcin V antimicrobial peptide is used to secrete an antimicrobial peptide that does not include the Microcin V secretion tag, e.g., the Microcin V secretion machinery will identify, process, and secrete other antimicrobial peptides. The structure of a secretion tag that can be identified, processed, and secreted by the Microcin V secretion machinery include an amino acid sequence, often 15 to 24 amino acids in length, with a glycine-glycine or glycine-alanine motif at the two residues on the amino-terminal side of the processing site. When an antimicrobial peptide can be exported by the Microcin V secretory proteins, either because it is modified to include a Microcin V signal peptide or is identified, processed, and exported by the Microcin V secretory proteins, a vector that includes these antimicrobial peptides can advantageously include only the coding regions encoding the Microcin V secretory proteins.

The heterologous promoter operably linked to the transcription unit encoding a polycistronic mRNA controls expression of the operably linked transcription unit. The promoter may be a controllable promoter, such as an inducible promoter or a repressible promoter, or the promoter may be a constitutive promoter. Examples of controllable promoters include promoters to which a modulating protein binds to inhibit or activate transcription in the presence of a modulating agent. Controllable promoters also include promoters which are activated when a microbe is present in an environment that includes a specific signal. Modulating proteins and modulating agents are described herein. Examples of such promoters include a chloride-inducible promoter and a tetracycline-inducible promoter. Such controllable promoters are known in the art and are routinely used to control expression of operably linked coding regions in prokaryotes. Such promoters often include −35 and −10 regions with an operator site also present, typically between the −35 and −10 regions. Variants of known controllable promoters may be produced and used as described herein.

An example of a useful chloride-inducible promoter is shown at SEQ ID NO: 1. The chloride-inducible promoter has been reported to be active at 0.1 M chloride, which is within the relevant range in the gut (˜0.05-0.15 M); however, it is also regulated by other environmental cues, including glutamate and pH (Sanders et al., 1998, Mol. Microbiol., 27(2):299-310; Sanders et al., U.S. Pat. No. 6,140,078), thus, while this type of promoter senses the environment, prior to the experiments presented herein it was unclear if the chloride-inducible promoter would drive expression in the complex environment present in the gut of an animal. A chloride-inducible promoter is obtainable from Lactococcus lactis.

(SEQ ID NO: 1)
AGATCTCAAATAAAAAGAGTTGGTTGAGATTTCAACTAGCT
CTTTTTATTTTAAATTGGTAGCTGAGCGTTGTATAAGCTTT
TATGTCTTTCTATATCAACTTTTAATAGAAATATAAAGTAA
TATAAATGTTTTTATAATAAATTATGTGAGATATATTTTTT
TGTCCGTACTGGTATAGATTTGACGATTAAGTCTTAAATAA
GTTATAATCTCAATTGCGTAATTTCTTAAATACAGAAATAA
CAACTACATTGGTAGACTGATTAAAAAGTGTACTTGATGAA
CTGTTATAAACCTTAAAAAAATAAAAATAATAGTTTGGGGG
ATGTTAAAGATGTATAAAAAATATGGAGATTGTTTTAAAAA
GTTGCGAAACCAAAAGAATTTAGGGTTATCATACTTTAGTA
AACTTGGAATAGACCGTTCAAATATATCTAGATTTGAACAT
GGAAAATGTATGATGAGTTTTGAGCGTATAGATTTGATGTT
AGAAGAAATGCAAGTTCCGTTATCTGAGTACGAATTGATTG
TAAATAATTTTATGCCGAATTTCCAAGAATTTTTTATATTA
GAATTGGAAAAAGCTGAATTTAGCCAAAATCGAGATAAAAT
AAAAGAGTTGTATTCTGAGGTCAAAGAAACGGGGAATCATT
TACTGACGGTTACCGTGAAAACGAAGCTTGGGAATATAAGT
CAGACAGAAGTTAAAGAAATTGAAGCTTATCTTTGCAATAT
TGAAGAGTGGGGATATTTTGAACTTACTTTATTTTATTTTG
TATCTGATTATCTCAATGTCAATCAATTAGAATTGCTGCTT
TTTAATTTTGATAAAAGATGTGAAAATTACTGTAGAGTCTT
AAAATATAGAAGGAGACTATTGCAAATAGCCTATAAAAGTG
TTGCGATATACGCGGCTAAAGGAGAAAGAAAAAAAGCCGAA
AATATTTTAGAAATGACTAAAAAATATCGAACTGTGGGAGT
CGATTTATATTCAGAAGTATTAAGACATCTTGCTAGAGCTA
TCATTATTTTTAATTTTGAAAATGCAGAGATTGGGGAAGAA
AAAATAAATTATGCTCTTGAGATTTTGGAAGAATTTGGAGG
AAAGAAGATAAAAGAATTCTATCAGAATAAAATGGAAAAGT
ATTTGAAAAGGTCAATTTAGTCTCTTTTGAGCTGTTGCTTT
AAAGCAACAGCTCAAAAGAGATTTTCTTTATTCTAGAGCAT
ATACTAGAGGGTGAAGATAGGTTGTCTGAAGCATTATAACT
TGTCTTTTAAAAAATTCAATCATAAATATAAGGAGGTATGC
CATGG

Examples of other useful promoters include those described in Volzing et al. (ACS Chem. Biol., 2011, 6:1107-1116) and Kaznessis et al. (WO 2014/052438).

A vector may optionally include another coding region that is operably linked to a second promoter, where the coding region encodes a protein (e.g., a modulatory protein) that regulates the expression of the heterologous promoter operably linked to a transcription unit, such as one encoding a polycistronic mRNA. A modulating protein is a protein that modulates expression of a transcription unit operably linked to a heterologous promoter described herein. In one embodiment, a modulating protein binds to a promoter or to other nucleotides around the promoter and modulates expression of a coding region operably linked to that promoter. In one embodiment, the modulating protein may either induce or prevent expression of the operably linked coding sequence in the presence of a modulating agent. An example of a modulating protein that controls expression of a chloride-inducible promoter is the activator protein GadR, and an example of a GadR protein is shown at SEQ ID NO:2 (Genbank Accession No. ADJ60177).

(SEQ ID NO: 2)
MYKKYGDCFKKLRNQKNLGLSYFSKLGIDRSNISRFEHGKCMMSFERIDL
MLEEMQVPLSEYELIVNNFMPNFQEFFILELEKAEFSQNRDKIKELYSEV
KETGNHLLTVTVKTKLGNISQTEVKEIEAYLCNIEEWGYFELTLFYFVSD
YLNVNQLELLLFNEDKRCENYCRVLKYRRRLLQIAYKSVAIYAAKGERKK
AENILEMTKKYRTVGVDLYSEVLRHLARAIIIENFENAEIGEEKINYALE
ILEEFGGKKIKEFYQNKMEKYLKRSI.

Examples of modulating proteins that bind to a tetracycline-inducible promoter are known in the art and include tetracycline repressor proteins, and reverse tetracycline repressor proteins. In one embodiment, modulator proteins are PROTEON and PROTEOFF, described in Volzing et al. (ACS Chem. Biol., 2011, 6:1107-111). These proteins are designed to work with a synthetic promoter that optimizes interactions between domains in PROTEON and PROTEOFF to result in high levels of expression of operably linked transcription units. The PROTEON protein contains an inducible binding domain (the reverse tetracycline repressor rTetR), which binds to a tetracycline operator sequence in the presence of an inducer such as tetracycline, doxycycline, or anhydrous tetracycline (aTc). PROTEOFF has the tetracycline repressor, TetR, instead of rTetR. rTetR and TetR undergo conformational changes upon binding to an inducer, which cause them to sensitively dissociate and associate from the tetracycline operator site, respectively. Thus, the TetR of PROTEOFF is designed to strongly bind to DNA in the absence of aTc and drive expression, and the aTetR of PROTEON is designed to strongly bind to DNA in the presence of aTc and drive expression.

The proteins described herein, including modulating proteins, may include conservative amino acid substitutions. In one embodiment, a protein described herein can include at least one, at least two, at least three, at least four, or at least five conservative substitutions. In one embodiment, a protein described herein is structurally similar to a reference protein, such as a modulatory protein, a CvaA protein, a CvaB protein, or an antimicrobial peptide.

When present, the second promoter controls expression of the operably linked coding region encoding a modulating protein. In one embodiment, the second promoter is a constitutive promoter. Examples of constitutive promoters include, but are not limited to, P23 and PnisA. Examples of these promoters are disclosed in Kaznessis et al. (WO 2014/052438). These and other constitutive promoters are known in the art and routinely used to control expression of operably linked coding regions in prokaryotes. Variants of known constitutive promoters may be produced and used as described herein.

In one embodiment, expression of the modulating protein is not driven by a second promoter, rather the coding region encoding the modulating protein is part of the polycistronic mRNA that encodes the antimicrobial peptides. In this embodiment, expression of the modulating protein is subject to a positive feedback mechanism that results in greatly increased expression of the modulating protein, and the antimicrobial peptides, in appropriate conditions. In one embodiment, the modulating protein that is expressed in the same polycistronic mRNA as the antimicrobial peptides is PROTEOFF or PROTEON, and the promoter driving expression of the polycistronic mRNA that encodes the modulating protein PROTEOFF or PROTEON and the antimicrobial peptides is a synthetic promoter similar or identical to the promoter described by Volzing et al. (ACS Chem. Biol., 2011, 6:1107-111).

Construction of vectors described herein may employ standard ligation techniques known in the art. See, e.g. (Sambrook et al., 1989. Molecular cloning : a laboratory manual, 2nd ed. Cold Spring Harbor Laboratory Press, Cold Spring Harbor, N.Y.). Selection of a vector depends upon a variety of desired characteristics in the resulting construct, such as a selection marker, vector replication rate, and the like. Vectors can be introduced into a host cell using methods that are known and used routinely by the skilled person. The vector may replicate separately from the chromosome present in the microbe, or the polynucleotide may be integrated into a chromosome of the microbe. A vector introduced into a host cell optionally includes one or more marker sequences, which typically encode a molecule that inactivates or otherwise detects or is detected by a compound in the growth medium. For example, the inclusion of a marker sequence may render the transformed cell resistant to an antibiotic, or it may confer compound-specific metabolism on the transformed cell. Examples of a marker sequence include, but are not limited to, sequences that confer resistance to kanamycin, ampicillin, chloramphenicol, tetracycline, streptomycin, neomycin, and erythromycin. Generally, introduction of a vector into a host cell, origin of replication, ribosomal sites, marker sequences, and other aspects of vectors may vary depending on whether the host cell is a Gram positive or a Gram negative microbe; however, these aspects of vector biology and heterologous gene expression are known to the skilled person and are routine. In one embodiment, a vector is pMPES. In addition to including different antimicrobial peptides, pMPES may be modified to include a different origin of replication, different marker sequences, or other modifications that are routine in the art of designing vectors,

Also provided herein are genetically modified microbes that include a vector disclosed herein. Compared to a control microbe that is not genetically modified in the same way, a genetically modified microbe will exhibit expression and secretion of an antimicrobial protein in the presence of a modulating agent or when it is in a suitable environment. In one embodiment, a genetically modified microbe may produce detectable levels of an antimicrobial peptide in the absence of an inducer; however, such levels of an antimicrobial peptide are substantially lower than the levels detectable in the presence of a modulating agent. A vector may be present in the microbe as a vector or integrated into a chromosome. In one embodiment, a portion of the vector is integrated into a microbe's chromosome.

In another embodiment, the positive feedback mechanism of protein expression may result in high level of antimicrobial peptides, even in the absence of a modulating agent. Use of a system that includes the PROTEON modulating protein encoded by the polycistronic mRNA was predicted to require an inducer to cause expression of the polycistronic mRNA; however, it was unexpectedly observed that such a system still resulted in high levels of expression of the polycistronic mRNA. Without intending to be limiting, it is believed that the promoter is leaky, e.g., it has a low level of expression of the PROTEON protein. The PROTEON protein results in such high levels of expression that even minor amounts of the PROTEON protein results in positive feedback and sufficient expression for use in the methods described herein.

Examples of useful bacterial host cells that may be modified to have a vector described herein include, but are not limited to, those that are normally part of the gastrointestinal microflora of an animal. Useful characteristics of a bacterial host cell include, for instance, resistance to bile-acids, generally recognized as safe (GRAS), suited to surviving passage to the gastrointestinal tract, and ability to colonize the gastrointestinal tract. Examples of useful bacterial hosts include, but are not limited to, lactic acid bacteria, including members of the Order Lactobacillales, such as Lactobacillus spp., (including, but not limited to, Lb. acidophilus, Lb. bulgaricus, Lb. reuteri, and Lb. plantarum), Lactococcus spp., (including, but not limited to, L. lactis), and Enterococcus spp.; members of the family Clostridiaceae, such as Clostridium spp.; members of the family Bifidobacteriaceae, such as Bifidobacterium spp.; and enterobacteria, such as E. coli, including E. coli Nissle 1917. In one embodiment, a bacterial host cell is probiotic microbe.

Also provided are methods of using the vectors and genetically modified microbes disclosed herein. The genetically modified microbe may be present in a composition, such as a pharmaceutically acceptable formulation. In one embodiment, a formulation may be a fluid composition. Fluid compositions include, but are not limited to, solutions, suspensions, dispersions, and the like. Fluid compositions may be incorporated in the water supply of a host. In one embodiment, a formulation may be a solid composition. Solid compositions include, but are not limited to, powder, granule, compressed tablet, pill, capsule, chewing gum, wafer, and the like. Solid compositions may be incorporated in the food supply of hosts. Those formulations may include a pharmaceutically acceptable carrier to render the composition appropriate for administration to a subject. As used herein “pharmaceutically acceptable carrier” includes pharmacologically inactive compounds compatible with pharmaceutical administration. The compositions may be formulated to be compatible with its intended route of administration. A composition may be administered by any method suitable for depositing in the gastrointestinal tract of a subject. Examples of routes of administration include rectal administration (e.g., by suppository, enema, upper endoscopy, upper push enteroscopy, or colonoscopy), intubation through the nose or the mouth (e.g., by nasogastric tube, nasoenteric tube, or nasal jejunal tube), or oral administration (e.g., by a solid such as a pill, tablet, or capsule, or by liquid).

For therapeutic use, a composition may be conveniently administered in a form containing one or more pharmaceutically acceptable carriers. Suitable carriers are well known in the art and vary with the desired form and mode of administration of the composition. For example, they may include diluents or excipients such as fillers, binders, wetting agents, disintegrators, surface-active agents, glidants, lubricants, and the like. Typically, the carrier may be a solid (including powder), liquid, or combinations thereof. Each carrier is preferably “acceptable” in the sense of being compatible with the other ingredients in the composition and not injurious to the subject. The carrier is preferably biologically acceptable and inert, i.e., it permits the composition to maintain viability of the biological material until delivered to the appropriate site.

Oral compositions may include an inert diluent or an edible carrier. For the purpose of oral therapeutic administration, the active compound can be incorporated with excipients and used in the form of tablets, troches, or capsules, e.g., gelatin capsules. Oral compositions can also be prepared by combining a composition of the present disclosure with a food. In one embodiment a food used for administration is chilled. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The active compounds can also be prepared in the form of suppositories (e.g., with conventional suppository bases such as cocoa butter and other glycerides) or retention enemas for rectal delivery.

The active compounds may be prepared with carriers that will protect the compound against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. Liposomal suspensions can also be used as pharmaceutically acceptable carriers. These can be prepared according to methods known to those skilled in the art.

In one embodiment, a composition may be encapsulated. For instance, when the composition is to be administered orally, the dosage form is formulated so the composition is not exposed to conditions prevalent in the gastrointestinal tract before the desired site, e.g., high acidity and digestive enzymes present in the stomach and/or upper intestine. The encapsulation of compositions for therapeutic use is routine in the art. Encapsulation may include hard-shelled capsules, which may be used for dry, powdered ingredients, or soft-shelled capsules. Capsules may be made from aqueous solutions of gelling agents such as animal protein (e.g., gelatin), plant polysaccharides or derivatives like carrageenans and modified forms of starch and cellulose. Other ingredients may be added to a gelling agent solution such as plasticizers (e.g., glycerin and or sorbitol), coloring agents, preservatives, disintegrants, lubricants and surface treatment.

In one embodiment, the method includes administering an effective amount of a genetically modified microbe to a subject in need of such a genetically modified microbe. The subject may be, for instance, a human, or avian (including, for instance, chickens or turkeys), bovine (including, for instance, cattle), caprine (including, for instance, goats), ovine (including, for instance, sheep), porcine (including, for instance, swine), equine (including, for instance, horses), canine (including, for instance, dogs), or feline (including, for instance, cats). The subject may be of any age. A subject can have a gastrointestinal microflora that requires modification, such as elimination or reduction of one or more microbes. For instance, a subject may have a microbial pathogen, such as a nosocomial pathogen, present in its gastrointestinal tract.

In one embodiment, the method may further include administering to the subject a modulating agent. In one embodiment, such a modulating agent will interact with the modulating protein, e.g., a tet-repressor, and result in expression of the coding sequence encoding the antimicrobial peptide. The modulating agent may be administered to the subject before, with, or after the administration of the genetically modified microbe, or a combination thereof.

In one embodiment, it is not necessary to administer a modulating agent to the subject. When the genetically modified microbe includes a vector that includes a promoter such as a chloride-inducible promoter, the microbe will begin to express the antimicrobial peptides when it is in a suitable environment, such as the gastrointestinal tract.

In one embodiment, a method includes reducing the number of pathogenic microbes. The method can include exposing a pathogenic microbe to a genetically modified microbe described herein that expresses at least two, or at least three antimicrobial peptides. The exposure of the pathogenic microbe to multiple antimicrobial peptides increases activity and efficacy and results in a greater reduction of the pathogenic microbe than exposure to just one antimicrobial peptide. The reduction can be at least 10-fold, at least 100-fold, or at least 1000-fold. The pathogenic microbe can be in vivo (e.g., inside or on the body of a subject) or in vitro (e.g., not in or on the body of a subject).

In one embodiment, a method includes reducing development of resistance to antimicrobial peptides. Microbes can develop resistance to an antibiotic such as an antimicrobial peptide, e.g., a population of microbes can include individuals that are resistant to an antibiotic. The Examples provided herein demonstrate that use of at least two, or at least three antimicrobial peptides reduces the development of resistance and the resulting regrowth of the pathogenic microbe after exposure to the antimicrobial peptides. Thus, a method can include exposing a pathogenic microbe to a genetically modified microbe described herein that expresses at least two, or at least three antimicrobial peptides. Exposure of the pathogenic microbe to at least two, or at least three antimicrobial peptides results an increase in the amount of time needed for regrowth of the pathogenic microbe, a measurement of development of resistance compared to the pathogenic microbe that is exposed to only one of the antimicrobial peptides. The increase in the amount of time needed for regrowth of the pathogenic microbe can be at least 12 hours, at least 24 hours, or at least 48 hours. The pathogenic microbe can be in vivo (e.g., inside or on the body of a subject) or in vitro (e.g., not in or on the body of a subject).

In one embodiment, a method includes treating a subject having a pathogenic microbe present in its gastrointestinal tract. In another embodiment, the present disclosure is directed to methods for treating certain conditions in a subject that may be caused by, or associated with, a microbe. Such conditions include, for instance, Gram negative microbial infections and Gram positive microbial infections of the gastrointestinal tract. Examples of conditions that may be caused by the presence of certain microbes in a subject's gastrointestinal tract include, but are not limited to, diarrhea, gastroenteritis, hemolytic-uremic syndrome, inflammatory bowel disease, irritable bowel disease, and Crohn's Disease.

Treating a subject, such as a subject having a pathogenic microbe or a subject having a condition, can be prophylactic or, alternatively, can be initiated after the need for treatment arises. Treatment that is prophylactic, for instance, initiated before a subject manifests symptoms of a condition caused by a pathogenic microbe, such as a member of the genus Salmonella, Clostridia, Klebsiella or Enterococcus, is referred to herein as treatment of a subject that is “at risk” of developing the condition. Typically, a subject “at risk” of developing a condition is a subject likely to be exposed to a pathogenic microbe, such as a member of the genus Salmonella, Clostridia, Klebsiella or Enterococcus, causing the condition. For instance, the subject is present in an area where the condition has been diagnosed in at least one other subject (e.g., a hospital in the case of a nosocomial infection). Accordingly, administration of a composition can be performed before, during, or after the occurrence of a condition caused by a pathogenic microbe. Treatment initiated after the development of a condition may result in decreasing the severity of the symptoms of one of the conditions, or completely removing the symptoms. In this aspect of the invention, an “effective amount” is an amount effective to prevent the manifestation of symptoms of a condition, decrease the severity of the symptoms of a condition, and/or completely remove the symptoms. The potency of a composition described herein can be tested according to routine methods (see, for instance, Stanfield et al., Microb Pathog., 3:155-165 (1987), Fox et al., Am. J. Vet. Res., 48:85-90 (1987), Ruiz-Palacios, Infect. Immun., 34:250-255 (1981), and Humphrey et al., J. Infect. Dis., 151:485-493 (1985)). Methods for determining whether a subject has a condition caused by a pathogenic microbe and symptoms associated with the conditions are routine and known to the art.

The microbe targeted by the antimicrobial peptide expressed and secreted by a genetically modified microbe may be a Gram negative or a Gram positive microbe. Examples of Gram negative microbes include, but are not limited to, Salmonella spp. (including Salmonella enterica serotypes typhimurium and enteritidis), E. coli (including E. coli O157:H7, enterotoxigenic E. coli, enteroinvasive E. coli, and enteropathogenic E. coli), Shigella spp., Campylobacter spp., Vibrio spp., and Klebsiella spp. Examples of Gram positive microbes include, but are not limited to, Staphylococcus spp. Listeria spp., Clostridia spp. (including Clostridia difficile and Clostridia perfringens) and Enterococcus, such as for instance, E. faecalis and E. faecium. In one embodiment, a target microbe is one that is resistant to an antibiotic, such as vancomycin (e.g., vancomycin resistant Enterococcus) and carbapenem (e.g., carbapenem resistant enterobacteriaceae, such as Klebsiella). Target microbes may be microbes transmitted to a subject through the ingestion of contaminated food, or by transmission from another subject (for instance, transmission from animal to animal, including animal to human).

In one embodiment, the antimicrobial peptides produced by the genetically modified microbe administered to the subject are directed to a specific type of microbe. For instance, in embodiments where a subject has an enterococcal infection of the GI tract, such an infection by one or more vancomycin-resistant Enterococcus, a genetically modified microbe administered to the subject can secrete antimicrobial peptides that target members of the genus Enterococcus, and limit reduction of useful commensal microbes present in the GI tract of the subject. Examples of such antimicrobial peptides include, but are not limited to, Enterocin A, Enterocin B, Enterocin P, and Hiracin JM79.

In another embodiment where a subject has a salmonella infection of the GI tract, a genetically modified microbe administered to the subject can secrete antimicrobial peptides that target members of the genus Salmonella, and limit reduction of useful commensal microbes present in the GI tract of the subject. Examples of such antimicrobial peptides include, but are not limited to, Microcin V, Microcin 24, and Microcin 25.

In another embodiment where a subject has a clostridia infection of the GI tract, a genetically modified microbe administered to the subject can secrete antimicrobial peptides that target members of the genus Clostridia, and limit reduction of useful commensal microbes present in the GI tract of the subject. Examples of such antimicrobial peptides include, but are not limited to, Endolysin 170 (Lys170), PlyV12, EFAL-1, ORF9, Lys168. Optionally, the method may further include administration of other therapeutic agents. In one embodiment a therapeutic agent may include one or more antibiotics. The antibiotic used depends on the target microbe and its antibiotic sensitivity profile. In one embodiment a combination of antimicrobial peptides and antibiotic resulted in an unexpected decrease in numbers of target microbe. Also observed was an increase in the amount of time needed for regrowth of the target microbe, a measurement of development of resistance. Specifically, Rifampicin alone had no noticeable reduction in E. faecium growth. Enterocin A, Enterocin P, and Hiracin JM79 were able to drastically reduce counts of E. faecium but a resistant subpopulation immediately arose. Interestingly, the combination of rifampicin with these three peptides maintained this high level of activity but prevented the regrowth of resistant mutants. These results were not observed when rifampicin was replaced with streptomycin or ampicillin. Thus, in one embodiment treating a subject having a pathogenic microbe includes administering a genetically modified microbe having a vector that includes at least three or at least four antimicrobial peptides selected from a class IIa, such as Enterocin A, a class IIb such as AS-48, and a class I, such as Nisin, along with an antibiotic that inhibits bacterial DNA-dependent RNA synthesis due to high affinity for the prokaryotic RNA polymerase. In another embodiment, treating a subject having a pathogenic microbe includes administering a genetically modified microbe having a vector that includes at least three or at least four antimicrobial peptides that target mannose phosphotransferase, such as Enterocin A, Enterocin P, and Hiracin JM79, along with an antibiotic that inhibits bacterial DNA-dependent RNA synthesis due to high affinity for the prokaryotic RNA polymerase. Examples of this type of antibiotic includes rifamycins, such as rifampicin, rifabutin, rifapentine, and rifalazil.

The method may further include determining whether at least one symptom associated with a condition cause by a target microbe is reduced, and/or determining whether the shedding of the target microbe by the subject is reduced. Methods for determining whether a subject has a reduction in a symptom associated with a condition are routine and known in the art. Methods for measuring shedding of a microbe are likewise routine and known in the art.

The present disclosure is illustrated by the following examples. It is to be understood that the particular examples, materials, amounts, and procedures are to be interpreted broadly in accordance with the scope and spirit of the invention as set forth herein.

### EXAMPLE 1

### A Chloride-Inducible Expression Vector for Delivery of Antimicrobial Peptides Against Antibiotic-Resistant Enterococcus faecium

### Abstract

Antibiotic-resistant enterococcal infections are a major concern in hospitals where patients with compromised immunity are readily infected. Enterococcus faecium is of particular interest as these pathogens account for over 80% of vancomycin-resistant enterococcal infections. Antimicrobial peptides (AMPs) produced at the site of infection by engineered bacteria may offer a potential alternative to traditional antibiotics for the treatment of resistant bacteria such as E. faecium. For this mode of delivery to be effective, it is helpful to identify a suitable protein expression system that can be used in the desired delivery bacterium. This study describes a promising chloride-inducible promoter and its application in the bacterial delivery of AMPs from Lactococcus lactis to reduce counts of E. faecium in vitro. Reporter gene studies show that at chloride concentrations found within the human intestines, the chloride-inducible promoter exhibits surprisingly high protein expression compared to the commonly-used nisin-inducible promoter. These results indicate that this system is powerful and would not require the exogenous administration of an inducer molecule. In its application for AMP production against E. faecium in vitro, L. lactis producing AMPs under the chloride promoter rapidly decreased E. faecium counts by nearly 10,000 fold. As an extension of this application, the potential in using this type of delivery system in combination with traditional antibiotics to slow the development of resistance is also demonstrated. Collectively, this study shows the promise of using a chloride-inducible promoter for the bacterial delivery of AMPs in the body for the treatment of vancomycin resistant enterococci (VRE) and other antibiotic-resistant bacteria.

Enterococci infections are a rising concern for healthcare due to the increasing frequency of multidrug resistant cases. As of 2013, nearly 30% of all reported enterococcal infections were antibiotic resistant (Frieden, 2013. Antibiotic Resistance Threats in the United States U.S. Department of health and human services centers for disease control and prevention). This high percentage of resistance is especially disconcerting in hospitals because patients with compromised immune systems or patients who are on antibiotic regiments are particularly susceptible to enterococcal infections (Arias and Murray, 2012, Nat. Rev. Microbiol. 10:266-78). Once an infection has occurred, it can be difficult to eradicate from not only the infected patient but from the entire hospital environment. Antibiotic resistance makes this process even more challenging and these infections become both more dangerous and costly (Neely and Maley, 2000, J. Clin. Microbiol. 38:724-6).

Enterococcus faecium and Enterococcus faecalis comprise nearly all vancomycin-resistant enterococcal infections (Arias et al., 2012, Clin. Infect. Dis. 54 Suppl 3:S233-8). While E. faecalis is more prevalent as an infectious agent, E. faecium is more commonly resistant to antibiotics than E. faecalis, and is known for its ability to rapidly transfer antibiotic resistance (Arias and Murray, 2012, Nat. Rev. Microbiol. 10:266-78). For example, nearly 81% of E. faecium infections are considered vancomycin-resistant (VR) compared to only 5% of E. faecalis infections (Arias et al., 2012, Clin. Infect. Dis. 54 Suppl 3:S233-8). Additionally, E. faecium carrying resistance to vancomycin is also commonly resistant to many first-line antibiotics including both β-lactams and aminoglycosides (Marothi et al., 2005, Indian J. Med. Microbiol. 23:214-9). It is thus important to find a means of treating these pathogens as health care providers are often left with limited options for the treatment of vancomycin resistant enterococci (VRE) infections.

Antimicrobial peptides (AMPs) may offer a potential alternative to traditional antibiotics for the treatment of VRE infections. Bacteriocins, a class of AMPs, are short peptides naturally produced by many bacteria as a means of eliminating competing microbes. For example, the bacteriocins Enterocin A, Enterocin P, and Hiracin JM79 are known to be highly active against a wide array of enterococci, including both vancomycin-resistant E. faecalis and E. faecium (Borrero et al., 2015, ACS Synth. Biol. 4(3):299-306). While bacteriocins are often extremely potent against their target bacteria, their activity is typically species-specific, thus making them less destructive to the native microbiota compared to traditional antibiotics (Kjos et al., 2011, Microbiology 157:3256-67). In addition to being less destructive, this narrow spectrum activity may also avoid unnecessary pressure for resistance development among the unaffected surrounding microbes.

Despite their demonstrated efficacy against many pathogens of interest, AMPs are limited in their application for treatment of internal infections because oral and intravenous delivery are hindered due to the fast degradation of the peptides in the body (Park et al., 2011, Int. J. Mol. Sci. 12:5971-92). Because many infections, including those caused by VRE, are often initiated in the GI tract, it is necessary to find a means of delivery AMPs to the intestines (Arias and Murray, 2012, Nat. Rev. Microbiol. 10:266-78).

It may be possible to deliver these peptides to the site of infection using engineered probiotic bacteria. Lactococcus lactis has been previously selected as a delivery vehicle because it has been shown to survive the human gastrointestinal (GI) tract and is considered a potentially probiotic organism (Klijn et al., 1995, Appl. Environ. Microbiol. 61:2771-4; Drouault et al., 2002, Appl. Environ. Microbiol. 68:3166-8). Indeed, these bacteria have already been successfully used phase I clinical trials for the delivery of transgenic proteins for the treatment of Crohn's disease in the human intestine (Braat et al., 2006, Clin. Gastroenterol. Hepatol. 4:754-9).

In a previous study, the bacteriocins Enterocin A, Enterocin P, and Hiracin JM79 were successfully produced in L. lactis NZ9000 under the E. faecalis responsive promoter, PrgX-PrgQ. Borrero and co-workers demonstrated that the expression system was highly effective at both targeting and decreasing E. faecalis (Borrero et al., 2015, ACS Synth. Biol. 4(3):299-306). While the PrgX-PrgQ AMP expression system may be a candidate for the treatment of E. faecalis, E. faecium does not produce the inducer molecule, the sex pheromone peptide cCF 10, which is required to activate the PrgX-PrgQ promoter. The use of the PrgX-PrgQ expression system would thus require the exogenous application of cCF 10 when treating E. faecium or other non-cCF10 producing strains of Enterococcus (Borrero et al., 2015, ACS Synth. Biol. 4(3):299-306).

This study characterizes and implements a general AMP expression system against enterococci that can be induced by the conditions found inside the gut. The use of an environmentally-inducible promoter is valuable in that it may be used in the treatment of any pathogen.

For this study, we have selected the lactococcal chloride-inducible promoter, previously discovered by Sanders et al. (Sanders et al., 1997, Appl. Environ. Microbiol. 63:4877-82). The chloride-inducible promoter is believed to be controlled by the activator protein GadR. The ability of GadR to turn on the promoter is thought to be dependent on the environmental chloride concentration, though the mechanism behind this interaction remains unclear (Sanders et al., 1997, Appl. Environ. Microbiol. 63:4877-82).

In this study the chloride-inducible promoter was characterized then compared to the widely-used nisin-inducible promoter using reporter protein assays. The chloride-inducible promoter was then used to express the three bacteriocins Enterocin A, Enterocin P, and Hiracin JM79 in L. lactis subsp. cremoris NZ9000. The ability of the chloride-inducible AMP expression system to inhibit a variety of both E. faecium and E. faecalis strains was tested using agar diffusion assays. E. faecium inhibition was further quantified using liquid co-culture tests. Lastly, as an extension of this project, combination treatments using traditional antibiotics and bacterial AMP delivery were explored as a means of reducing the development of AMP resistance.

### Materials and Methods

### Bacterial Strains and Growth Conditions

Bacteria used in this study are listed in Table 4. L. lactis NZ9000 was cultured at 32° C. in M17 broth (Oxoid Ltd., Basingstoke, U.K.) supplemented with 0.5% (w/v) glucose (GM17). E. faecium 8-E9 was grown in BHI broth (Oxoid) at 37° C. E. coli MC1061 F′ was grown in LB broth (Fisher Scientific, Fair Lawn, N.J., U.S.A.) at 37° C., with shaking. Agar plates were made by the addition of 1.5% (wt/vol) agar (Oxoid) to the liquid media. When necessary, rifampicin (Sigma Chemical Co., St. Louis, Mo., U.S.A.) was added to the media at 5 μg/mL for E. faecium, and chloramphenicol (Mediatech Inc., Manassas, Va., U.S.A.) at 5 μg/mL or 20 μg/mL, for L. lactis or E. coli, respectively.

TABLE 4
Bacteria Used in this Study

| Bacteria Strain | Description | Source |
|---|---|---|
| Lactococcus | plasmid-free strain, derivative of MG1363; pepN::nisRK, | Mobitec |
| lactis NZ9000 | nonbacteriocin producer | |
| Enterococcus | Ampicillin/Vancomycin/Linezolid resistant | University |
| faecium 8-E9 | | of MN |
| Enterococcus | Ampicillin/Vancomycin/Linezolid resistant | University |
| faecium 6-E6 | | of MN |
| Enterococcus | Ampicillin/Vancomycin/Linezolid resistant | University |
| faecium 7A | | of MN |
| Enterococcus | Ampicillin/Vancomycin/Linezolid resistant | University |
| faecium 9B | | of MN |
| Enterococcus | ATCC 47077; plasmid-free, Rifampicin/Fusidic Acid resistant | University |
| faecalis OG1RF | mutant of OG1; common laboratory strain | of MN |
| Enterococcus | ATCC 700802; First isolated Vancomycin-resistant and first | University |
| faecalis V583 | sequenced E. faecalis genome | of MN |
| Enterococcus | Gentamycin/Kanamycin/Streptmycin/Tetracycline/Erythromycin/ | University |
| faecalis Ch116 | Penicillan resistant, β-lactamase-producing isolate | of MN |
| Enterococcus | Rifampicin/Fusidic Acid resistant mutant; common laboratory | University |
| faecalis JH2-2 | strain | of MN |
| Enterococcus | Panose 7; fecal sample of healthy volunteer | University |
| faecalis Pan-7 | | of MN |
| Enterococcus | fecal sample of healthy volunteer | University |
| faecalis Com-1 | | of MN |
| Enterococcus | ATCC 14508; pAD1, pAMα 1; Erythromycin/tetracycline resistant | University |
| faecalis DS5 | strain | of MN |
| Escherichia coli | plasmid-free, recA+, non-amber suppressor strain | Lucigen |
| MC1061 F′ | | |

### Construction of Plasmids

Plasmids were constructed using standard molecular cloning techniques. All restriction enzymes were purchased from New England Biolabs (Beverly, Mass., U.S.A.). Fragments obtained and plasmids used are listed in Table 5.

TABLE 5
Plasmids and DNA Fragments Used in this Study

| | Description | Source |
|---|---|---|
| Plasmids | | |
| Geneart- | Kan^{r}; source of the chloride-inducible | Geneart |
| chloride | promoter system (CIP) | |
| pNZ8048 | Cm^{r}; inducible expression vector carrying | (1) |
| | the nisA promoter | |
| pNZC | pNZ8048 derivative containing the CIP | This work |
| pNZ8048L | pNZ8048 derivative containing lacZ under the | This work |
| | nisin-inducible promoter | |
| pNZCL | pNZC derivative containing lacZ under the CIP | This work |
| pBac | Spc^{r}; source of Bac fragment | (2) |
| pNZCA3 | pNZC derivative containing Bac under the CIP | This work |
| pBK1 | Cm^{r}; source of lacZ | (2) |
| Fragments | | |
| chloride- | 1,317 bp fragment containing the chloride- | (3) |
| inducible | inducible promoter (P_{gad}) and the gene | |
| promoter | encoding the activator protein (gadR) | |
| system | under the control of a constitutive | |
| (CIP) | promoter (P_{gadR}) | |
| Bac | 1,610 bp fragment containing the enterocin | (2) |
| | A structural gene (entA) with its immunity | |
| | gene (entiA), the enterocin P structural | |
| | gene (entP) with its immunity gene (entiP), | |
| | and the hiracin JM79 structural gene | |
| | (hirJM79) with its immunity gene (hiriJM79) | |

(1) Kuipers et al., 1998, J. Biotechnol. 64: 15-21;
(2) Borrero et al., 2015, ACS Synth. Biol. 4(3): 299-306;
(3) Sanders et al., 1998, Mol. Microbiol. 27: 299-310.

The chloride-inducible promoter sequence (CIP) used in this work was adapted from Sanders and coworkers (Genebank Accession number AF005098, base pairs 821-2,071) (Sanders et al., 1998, Mol. Microbiol. 27:299-310) and synthesized by Geneart (Thermo Fisher Scientific). The sequence was then amplified using primers Chloride-F (5′-CGAATTGAAGGAAGGCCG, SEQ ID NO:33) and Chloride-R (5′-GCAGTGAAAGGAAGGCC, SEQ ID NO:34). The PCR fragment and plasmid pNZ8048 were both digested with restriction enzymes Bg/II and NcoI (New England Biolabs) and ligated at 16° C. for 16 hours. The resulting ligation was then transformed into electrocompetent E. coli MC1061 F′ (Lucigen). Successful transformants were identified by colony PCR using the primers pNZ8048-F (5′-GCCCCGTTAGTTGAAGAAGG, SEQ ID NO:35) and pNZ8048-R (5′-CAATTGAACGTTTCAAGCCTTGG, SEQ ID NO:36) and further verified by sequencing analysis. The resulting plasmid, pNZC, was isolated from E. coli using a QIAprep Miniprep kit (Quiagen) and then transformed into electrocompetent L. lactis NZ9000 (Holo and Nes IF. 1995. Methods Mol. Biol. 47:195-9).

For β-gal reporter gene studies, the lacZ reporter gene (lacZ) was inserted downstream of the chloride-inducible promoter. lacZ was amplified using the primers LacZ-F (5′-GCTAAGCCATGGAAG TTACTGACGTAAGATTACGG, SEQ ID NO:37) and LacZ-R (5′-TCGACTAGTTTATTATTATTTTTGACACCAGACCAACTGG, SEQ ID NO:38) from pBK1. Both the PCR products obtained and pNZC were then digested with restriction enzymes NcoI and SpeI, ligated, and transformed into E. coli MC1061 F′ and L. lactis as described above. The resulting plasmid is referred to as pNZCL. The plasmid pNZ8048L was created for these studies using an identical procedure to that used to create pNZCL. In this case however, pNZ8048 rather than pNZC was used for the backbone.

For AMP production, the genes encoding Enterocin A, Enterocin P, and Hiracin JM79 along with their immunity proteins (Fragment Bac from Table 5) were inserted into pNZC. Fragment Bac was amplified using primers AMP-F (5′-CATAACATGTCTACTATGAAAAAAAAGATTATCTC, SEQ ID NO:39) and AMP-R (5′-CACTAGTTTATCAAAGTCCCGACC, SEQ ID NO:40), using pBac as the template. pNZC was then digested with NcoI and SpeI while Bac was digested using PciI and SpeI. The digestion products originated were then ligated and transformed into E. coli MC1061 F′. The resulting plasmid, pNZCA3 was then transformed into electrocompetent L. lactis NZ9000.

### Beta-Galactosidase Assays

L. lactis NZ9000 containing pNZCL or pNZ8048L were grown overnight in GM17. The following day, cells were re-inoculated into fresh GM17 at an OD₆₀₀ of 0.15. Cells were then grown at 37° C. to an OD₆₀₀ of 0.4-0.5 at which point they were induced. L. lactis NZ900-pNZCL was induced by adding NaCl to the media to obtain final concentrations of 0.01, 0.05, 0.1, 0.3, and 0.5 M Cl⁻ and L. lactis NZ9000-pNZ8048L was induced by the addition of 5 ng/mL or 40 ng/mL nisin A as previously described (Volzing et al., 2013, ACS Synth. Biol. 2:643-50). After induction, OD₆₀₀ readings and 1 mL samples were collected each hour. Upon collection, samples were centrifuged for 7 minutes at 5,600×g, supernatant was removed, and the pellets were refrigerated until further analysis.

To measure β-gal activity we used the traditional Miller assay (Kozlowicz et al., 2004, Mol. Microbiol. 54:520-32) with some minor modifications. First, pellets were resuspended in 990 μL of Z-buffer (60 nM Na₂HPO₄.7H₂O (Sigma), 40 mM NaH₂PO₄.H2O (Sigma), 10 mM KCl (Sigma), 1 mM MgSO₄.7H₂0 (Sigma), 50 mM β-mercaptoethanol (Sigma), in DI water). A 105 μL aliquot of each sample was transferred to a polypropylene 96 well plate. Toluene (20 μL) was then added to each well and the plate was shaken for ˜30 seconds. The plate was then covered and incubated for 15 minutes at 32° C. 100 μL of 10 mg/mL ortho-Nitrophenyl-β-galactoside (ONPG) (Research Products Int'l. Corp., Mt. Prospect, Ill., U.S.A) dissolved in Z-buffer was then added to each well. The plate was covered again then incubated at 32° C. for 5-15 minutes until sufficient color had developed. 125 μL of 2 M Na₂CO₃ was then added to the wells to stop the reaction. Lastly, the plate was centrifuged at 4° C. for 30 minutes at 6,130×g to remove cell debris and the supernatant was transferred to a clean 96 well plate and the OD₄₂₀ and OD₅₅₀ was read using a plate reader (Synergy H1 Multi-Mode Reader; BioTek, Winooski, Vt.). The following formula was used to convert to activity units:

1 unit=1000*(Abs₄₂₀−1.75*Abs₅₅₀)/(incubation time (minutes)*sample volume (0.105 mL)*OD₆₀₀)

In this equation, Abs₄₂₀=OD₄₂₀ sample*0.734+OD₄₂₀ water*0.266 to adjust the final reaction concentration to that of the traditional Miller assay (Kozlowicz et al., 2004, Mol. Microbiol. 54:520-32).

### Supernatant Production

L. lactis containing pNZCA3 was grown overnight in BHI. Cells were then re-inoculated in fresh BHI at an OD₆₀₀ of 0.07-0.1. Because BHI contains ˜0.15 M NaCl, no additional salt was added for induction. Cells were then grown ˜6 hours then cell-free culture supernatant was obtained by centrifugation of culture at 12,000×g at 4° C. for 10 min, filtered through 0.2 μm pore-size filters (Whatman Int. Ltd., Maidstone, UK), and stored at −20° C. until use.

### Agar Diffusion Tests

L. lactis was grown on GM17 plates supplemented with chloramphenicol to produce single colonies while enterococci were grown overnight in BHI broth. The following morning, BHI supplemented with agar (0.8%) was inoculated with a 0.005% of the overnight enterococcal culture and poured into a petri dish. Once the plate had solidified, L. lactis colonies were stabbed into the semisolid media and the plates were incubated overnight at 37° C. Inhibition was confirmed by the formation of clear zones around the recombinant L. lactis strains.

### Co-culture and Supernatant Activity Assays

### Salt Concentration Tests

E. faecium was grown overnight in GM17 at 37° C. and L. lactis- pNZCA3 was grown in GM17 at 32° C. The following morning, 30 μL of L. lactis and E. faecium overnights were used to inoculate 5 ml fresh GM17 supplemented with the specified concentration of sodium chloride. This resulted in ˜1:2 ratio of E. faecium to L. lactis. GM17 was used for these experiments because of the lower salt concentration compared to BHI. Samples of the cultures were then taken at different times, serial diluted, and plated (10 μL of each dilution) on GM17 plates containing 5 μg/mL rifampicin (GM17-Rif) or 5 μg/mL chloramphenicol (GM17-Cm) and incubated overnight at 37° C. for E. faecium and 32° C. for L. lactis. The following day E. faecium or L. lactis colony forming units (CFU) were counted on GM17-Rif and GM17-Cm plates, respectively.

Combination with Rifampicin Tests

E. faecium was grown overnight in BHI at 37° C. and L. lactis-pNZCA3 was grown in BHI at 32° C. E. faecium was then inoculated at an OD₆₀₀ of ˜0.06 into 5 ml fresh BHI broth, and allowed to grow to an OD₆₀₀ of ˜0.1. For antibiotic experiments, 50 μg/mL streptomycin (Chem-Implex Int'l Inc., Wood Dale, Ill., U.S.A.), 100 μg/mL ampicillin (Sigma), or 30 μg/mL rifampicin (Gilbert et al., 2013, The Sanford Guide to Antimicrobial Therapy 2013 43 Pocth Edition|Rent 9781930808744|11930808747. Antimicrob. Ther.) were added to the fresh BHI broth. Cultures were then supplemented with 10% of the previously obtained supernatant or 10% of L. lactis-pNZCA3 culture at an OD₆₀₀ of ˜0.1 (time=0 hours). This resulted in ˜5:1 ratio of E. faecium to L. lactis. Samples of the cultures were then taken at different times, serial diluted, and plated (10 μL of each dilution) on GM17 plates containing 5 μg/mL rifampicin (GM17-Rif) or 5 μg/mL chloramphenicol (GM17-Cm) and incubated overnight at 37° C. The following day E. faecium or L. lactis colony forming units (CFU) were counted on GM17-Rif and GM17-Cm plates, respectively.

### Results

### Reporter Gene Studies

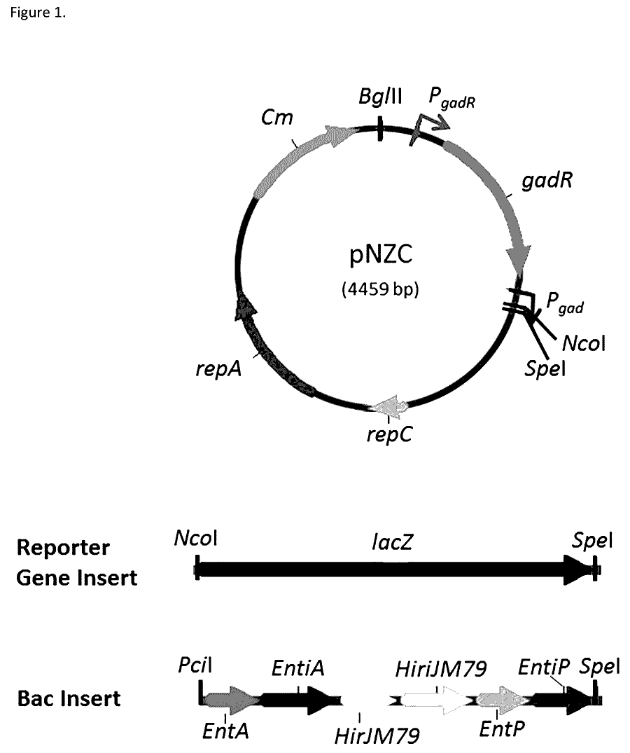

The promoter's dependence on chloride was characterized and it's activity within the range of chloride concentrations measured throughout the human GI tract (˜0.05-0.15 M) was verified (Fordtran et al., 1966, Am. J. Dig. Dis. 11:503-521). Additionally, it was desirable to compare the production under the chloride promoter to that of the widely-used nisin-inducible promoter to evaluate the strength of this expression system. To assess these parameters, the lacZ reporter gene was inserted under the control of the chloride-inducible promoter to create the plasmid pNZCL (FIG. 1) or under the nisin-inducible promoter in pNZ8048 to create the plasmid pNZ8048L. L. lactis NZ9000-pNZCL and L. lactis NZ9000-pNZ8048L were then grown to an OD₆₀₀ of ˜0.45 and induced with chloride (0.01 M, 0.05 M, 0.1 M, 0.3 M, 0.5 M) or nisin (0 ng/mL, 5 ng/mL, 40 ng/mL). Beta galactosidase production under the two promoters was then measured over time.

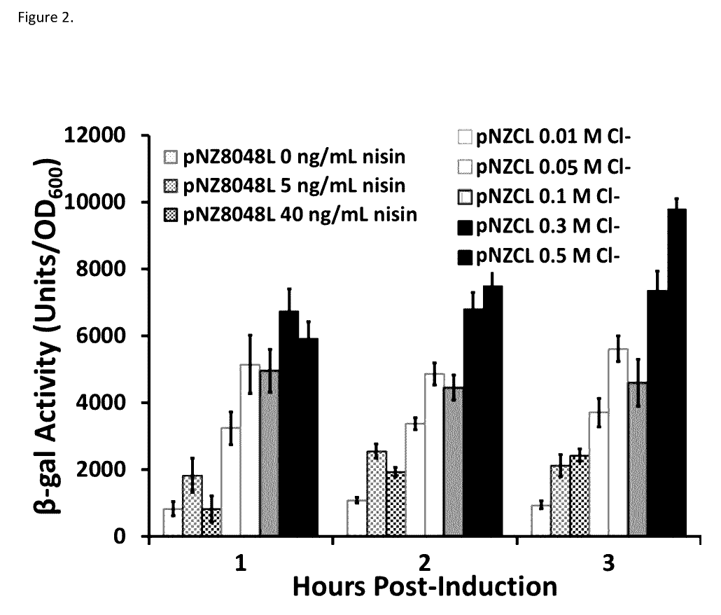

The results in FIG. 2 indicate that the chloride-inducible system is highly responsive to the chloride levels in the media and that within the range of physiological conditions (0.05 M-0.1 M), the chloride-inducible system expresses significantly more protein than the fully-induced nisin-promoter. It also appears that the production does not vary much within the induction range found in the intestines. The results presented here represent three technical replicates however, the experiments for FIG. 2 were repeated twice individually. Both trials showed that the chloride inducible system induced with 0.05 M and 0.1 M chloride showed higher β-gal production than the raisin promoter induced with either 5 or 40 ng/mL, nisin at all time points post-induction (P<0.005 t=1 and 2 hr, P<0.05 t=3 hr). Experiments comparing 0-gal expression under the chloride-inducible promoter have been repeated several times and showed similar trends in increased expression with increasing salt concentration. These results are promising for the use of this system for the delivery of proteins in the GI tract as they show high expression can be obtained from the promoter under the induction conditions naturally found within in the gut.

It should be noted that significant protein expression occurs even in GM17 medium without any added salt. The chloride concentration in GM17 was measured and found to be ˜0.01 M Cl⁻. Based on the trends observed in FIG. 2, it is possible to conjecture that the expression under the chloride promoter could be further reduced if the chloride concentration was lowered by using a different medium. This approach is used in the agar diffusion tests discussed below. However, a fully uninduced state (no chloride ions) cannot be obtained because chloride is necessary for bacterial survival.

### Delivery of AMPs Using the Chloride-Inducible Promoter

### Agar Diffusion Inhibition Tests

To apply the chloride inducible system for the production of AMPS, the three bacteriocin genes—enterocin A, enterocin P, and hiracin JM79 along with their immunity genes were inserted downstream of the chloride-inducible promoter to create the plasmid pNZCA3 (FIG. 1). L. lactis NZ9000 was then transformed with this plasmid and its antimicrobial activity was tested using an agar diffusion test using Enterococcus faecium 8-E9 as the indicator strain.

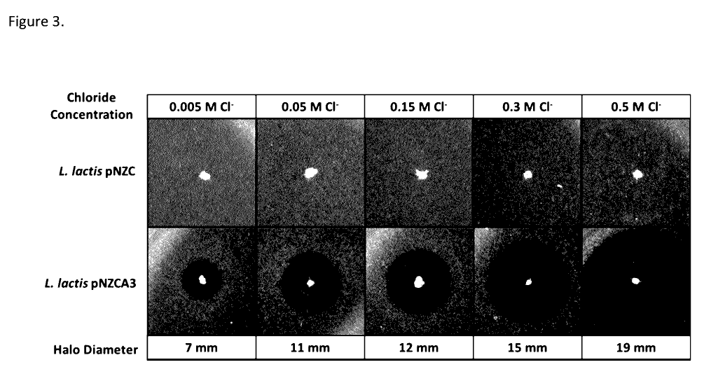

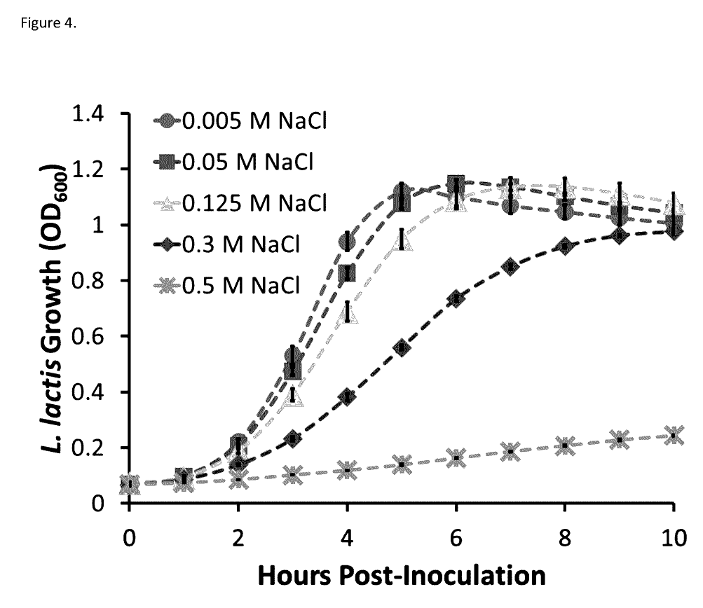

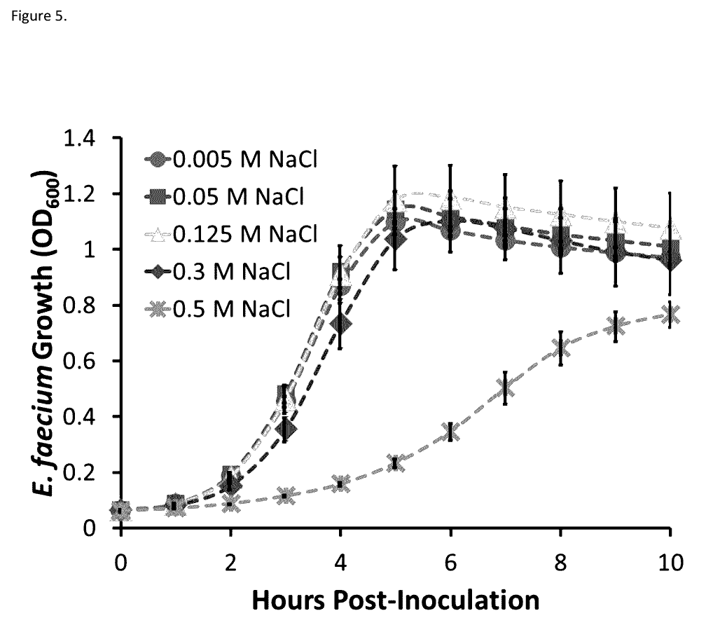

FIG. 3 shows the inhibition of E. faecium by L. lactis-pNZCA3 at different concentrations of chloride. For these studies, a modified GM17 medium was used which contains half the M17 in traditional GM17. This reduced the basal salt concentration from ˜0.01 M Cl⁻ to ˜0.005 M Cl⁻ which enabled a more nearly uninduced state. In this experiment, L. lactis-pNZC (chloride promoter, no AMPs) was used as a negative control. Based on the diameters of the halos produced, it appears that the overall AMP production is salt dependent as anticipated by the reported gene studies discussed above. Interestingly, there is a significant increase in diameter in 0.005 M and 0.05 M cultures but not in 0.05 M and 0.15 M. The halo sizes observed at 0.05 and 0.15 M in the modified GM17 are similar to those observed in traditional GM17 (10-12 mm). L. lactis and E. faecium growth curves in modified GM17 at different salt concentrations can be found in the Supplemental Material FIGS. 4 and 5 respectively. It should be noted that at 0.3 M and 0.5 M NaCl, E. faecium growth is significantly slowed (FIG. 5) which may contribute to the halo diameters at the higher salt concentrations.

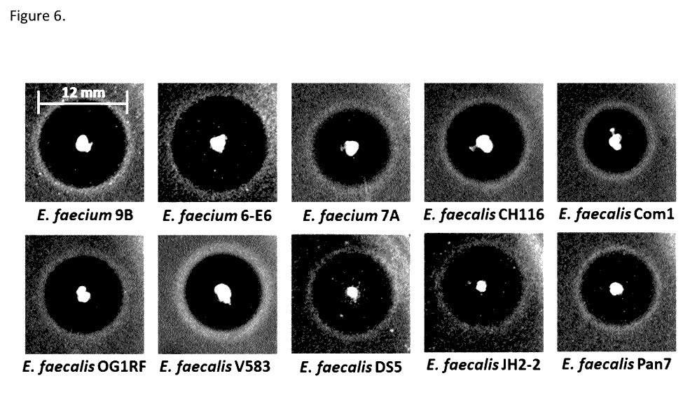

An important benefit of using an environmentally-inducible promoter for the delivery of AMPs is that, in principle, it can be used against any type of pathogen. To demonstrate that the chloride-promoter expressing the three AMPs Enterocin A, Enterocin P, and Hiracin JM79, can be used to target a broad range of enterococci, halo tests were performed against several strains of pathogenic, antibiotic-resistant strains of both E. faecium and E. faecalis. FIG. 6 shows the results of agar diffusion tests of L. lactis-PNZCA3 against 11 strains of enterococci (E. faecium 8-E9, not shown). In all cases we observed clear halos ˜8 mm-13 mm in diameter. All halo tests shown in FIG. 6 were done on BHI+agar (˜0.15 M Cl⁻). Activity was also tested against two E. faecalis isolates from healthy patients (Com1 and Pan7) indicating this system could potentially impact commensal enterococcal species.

### Liquid Co-Culture Inhibition Tests

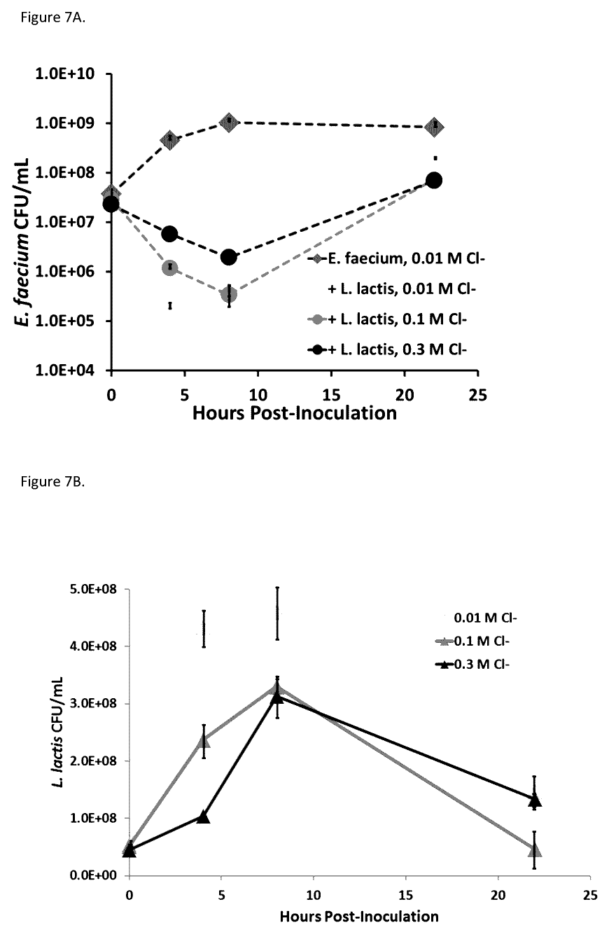

To further quantify the effect of the chloride-inducible AMP expression cassette on E. faecium growth, co-culture inhibition tests were done using E. faecium 8-E9 and L. lactis expressing Enterocin A, Enterocin P, and Hiracin JM79 under the chloride-inducible promoter. FIG. 7a shows the counts of viable E. faecium 8-E9 at different time points. E. faecium grown alone and E. faecium treated with AMP-producing L. lactis in GM17 with 0.01 M Cl⁻, 0.1 M Cl⁻, or 0.3 M Cl⁻ are shown. E. faecium growth has also been tested in the presence of L. lactis producing no AMPs (pNZC) and has been found to be nearly identical to normal E. faecium growth (data not shown). FIG. 7b shows the corresponding L. lactis counts in each culture. Interestingly, the highest inhibition of E. faecium by L. lactis-pNZCA3 co-culture was that of the 0.01 M culture. Similar results were observed in three individual experiments in which L. lactis induced with a higher concentration of chloride showed reduced inhibition of E. faecium. This is likely due to the faster growth of L. lactis at lower NaCl as seen in FIG. 7b. 4 hours post-induction, the counts of viable L. lactis were approximately 2 and 4-fold higher in the 0.01 M cultures compared to the cultures supplemented with 0.1 and 0.3 M NaCl, respectively. The difference in growth at increasing salt concentrations is significantly more pronounced than that observed when L. lactis is grown alone rather than in co-culture with E. faecium (FIG. 4).

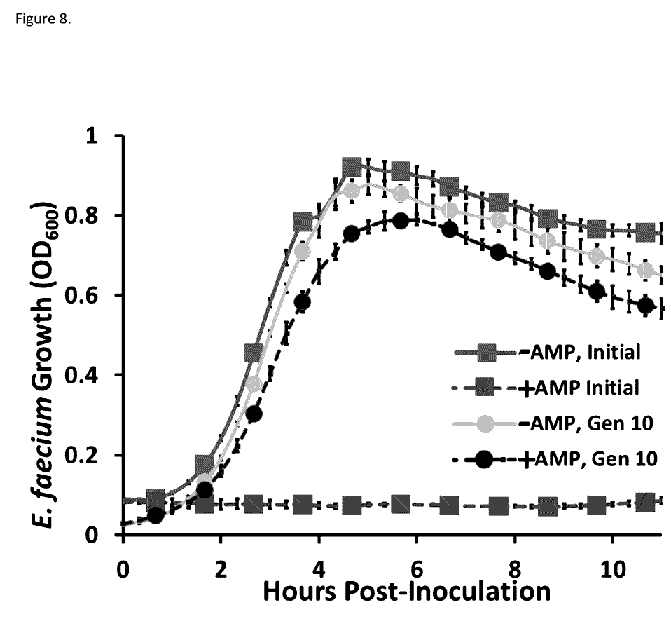

It is evident from FIG. 7a that even at non-optimal growth conditions, L. lactis is able to inhibit E. faecium immediately upon treatment. It is clear however that within 10 hours the pathogen begins to regrow. To test whether this was truly due to the appearance of E. faecium with stable resistance to the peptides or due simply to decreased AMP concentrations over time, E. faecium that arose from the culture treated with AMPs were regrown in fresh GM17 for ˜10 generations. Growth of the original culture and the re-grown resistant E. faecium was then monitored in GM17 with or without 10% L. lactis supernatant containing AMPs to determine if resistance was still present. FIG. 8 shows the growth curves of both the original E. faecium culture and the supposed resistant culture with and without AMPs. Even after growing 10 generations in the absence of AMPs, the resistant culture is only mildly impacted by the AMPs. These results indicate that the surviving E. faecium from the co-culture experiments are in fact stably resistant to the AMPs for at least 10 generations. The proposed mechanisms of this resistance are further discussed below.

Resistance Prevention using Class IIa Bacteriocins and Rifampicin Combined Treatment

Though L. lactis producing Enterocin A, Enterocin P, and Hiracin JM79 under the chloride-inducible promoter offers promise in temporarily decreasing E. faecium in co-culture, the rise in resistant mutants was concerning. To further demonstrate the potential of the AMP delivery system, we aimed to identify a means of combatting this resistance. It is well known that the combination of multiple antibiotics can help to postpone the development of antibiotic resistance (Lewis, 2013, Nat. Rev. Drug Discov. 12:371-87). Additionally, it has been observed in some cases that antibiotics as well as AMPs can act synergistically against the target pathogen (Rand et al., 2004, J. Antimicrob. Chemother. 53:530-2, Vaara and Porro, 1996, Antimicrob. Agents Chemother. 40:1801-5). One can thus imagine the potential in either producing additional AMPs from our currently engineered L. lactis or combining bacterial AMP delivery with traditional antibiotic treatments.

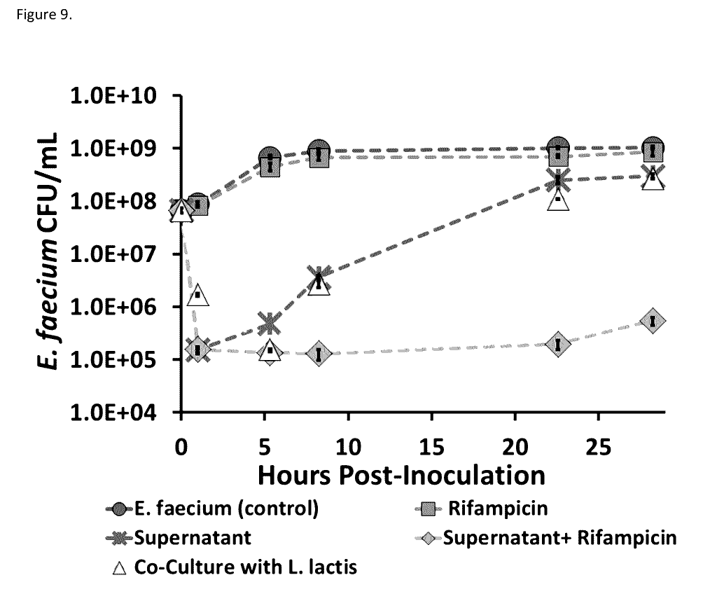

In an attempt to eliminate regrowth of E. faecium, several common antibiotics, including streptomycin, ampicillin, and rifampicin, were tested in combination with the three AMPs used in this study. Alone, none of these drugs showed significant activity against vancomycin-resistant E. faecium at clinically relevant concentrations (Gilbert et al., 2013. The Sanford Guide to Antimicrobial Therapy 2013 43 Pocth Edition|Rent 9781930808744|1930808747. Antimicrob. Ther) (FIG. 9, and data not shown for streptomycin and ampicillin). It was found however that treating E. faecium with a combination of rifampicin and the supernatant containing the three AMPs both decreased E. faecium counts by nearly four orders of magnitude and prevented regrowth of the pathogen for over 24 hours (FIG. 9). Data for FIG. 9 represent technical triplicates obtained from one of three individual tests. Similar decreases in E. faecium numbers were observed in all three tests and were consistent with values from several other colony counting experiments performed with this pathogen. The effect of rifampicin and rifampicin with AMPs on E. faecium is also representative of all three trials. Synergy of the AMPs with streptomycin and ampicillin was also tested and was found to be minor with these antibiotics (data not shown).

Because L. lactis used in these studies were not rifampicin-resistant, supernatant rather than co-culture was used with the antibiotic. A comparison between inhibition by 10% cell-free supernatant and 10% L. lactis-pNZCA3 demonstrates that similar stable inhibition might be obtained if rifampicin-resistant L. lactis-PNZCA3 rather than supernatant was used in combination with the antibiotic. In practice, these rifampicin-resistant L. lactis would be used in conjunction with the antibiotic.

### Discussion

We characterized and implemented a chloride-inducible expression system for the bacterial delivery of antimicrobial peptides. We demonstrated the efficacy of a previously discovered chloride-inducible promoter as a potentially useful expression system for the delivery of antimicrobial peptides by L. lactis. In this paper we have focused on developing a system to eliminate antibiotic-resistant E. faecium, however this type of expression system can easily be expanded with different AMPs to target a wide variety of pathogens.

The first section of the study focuses on characterizing protein expression under the chloride-inducible promoter using reporter gene assays. By comparing β-gal production between the chloride-promoter and the commonly-used nisin-inducible promoter, we found the chloride promoter to be a powerful expression system which is strongly activated at typical chloride concentrations found inside the human GI tract. These results were promising because they indicated that this promoter could be used to express AMPs (or other proteins) in the GI tract without additional induction. The reporter studies also showed that significant promoter activity was observed in the lowest-attained induction state (0.01 M Cl⁻).

In the future it may be of interest to further explore the responsiveness of the chloride-promoter to other environmental signals. For example, there is evidence that the promoter activity is also impacted by the glutamate concentration and pH of the culture (Sanders et al., 1998, Mol. Microbiol. 27:299-310). Glutamate availability, pH, or other environmental conditions could thus play an important role in the delivery of AMPs in the body and should be examined in the future. These variables can also provide additional parameters for improving promoter control for manufacturing and growth purposes.

The second portion of this study was to implement the chloride-inducible promoter for the production of AMPs against VRE. In a recent study from our lab, the E. faecalis-responsive PrgX-PrgQ promoter was used to express Enterocin A, Enterocin P, and Hiracin JM79 to eliminate E. faecalis (Borrero et al., 2015, ACS Synthetic Biology 4(3):299-306). While the PrgX-PrgQ system shows promise in targeting E. faecalis, it is not ideal for the treatment of E. faecium or other pathogens lacking the inducer pheromone cCF10 as these bacteria would not induce AMP production from the PrgX-PrgQ system. We thus sought an environmentally-inducible system that could be used for general AMP production against any pathogen.

To test the utility of this new expression system, the chloride promoter was used to express the same three AMPs (Enterocin A, Enterocin P, and Hiracin JM79) for the elimination of our indicator pathogen, E. faecium 8-E9, as well as a variety of other enterococcal strains at chloride concentrations obtainable in the intestines. It was shown that all 11 enterococcal strains tested were significantly inhibited by the lactococcal AMP delivery system. The wide-spectrum activity that can be obtained with this system demonstrates the potential benefit in using the chloride-inducible promoter for AMP delivery. Though this type of system will likely be effective in eliminating pathogenic enterococci, it was also observed that the two commensal E. faecalis strains were also inhibited by our AMPs. In the future, selection of AMPs with minimal impacts on the native gut microbiota may be helpful when using the chloride-promoter since promoter activity will not be localized to the pathogen.

In liquid co-culture tests, the system proved to be extremely effective in reducing E. faecium counts. Interestingly, it was seen that increasing salt concentration did not result in increased killing of the pathogen. Based on the colony counts of the L. lactis from the 0.01 M, 0.1 M, and 0.3 M cultures, this is likely impacted by reduced growth (and productivity) of L. lactis early in the co-culture. These results contrasted those observed in the agar diffusion tests which showed increased halo diameters at increased salt concentrations.

It should be noted that the impact of chloride-concentration in 0.01 M and 0.1 M cultures on L. lactis growth was far more pronounced in co-culture than in L. lactis cultures grown alone. These differences may be due to the competition for nutrients between L. lactis and E. faecium. We recognize that the nutrient availability and environmental stresses found within the GI tract are significantly different from those in vitro and that only in vivo tests can tell the true utility of this system. Based on the studies performed thus far however, high AMP production under the chloride promoter appears robust to growth and induction conditions which is invaluable for the proposed application.

The liquid co-culture tests also revealed that while the AMPs produced under the chloride-inducible expression system were initially very effective against E. faecium, resistance began to overtake the culture within 10 hours. Resistance was verified by monitoring growth of surviving bacterial in the presence and absence of AMPs. These results were not surprising as resistance to bacteriocins by L. lactis, E. faecalis, and Listeria monocytogenes has been previously reported (Kjos et al., 2011, Appl. Environ. Microbiol. 77:3335-42). Several mechanisms of resistance for class IIa bacteriocins have been hypothesized and explored. Most of these proposed mechanisms involve the mannose-phosphotranspherase (Man-PTS) system which is believed to be the receptor of these class IIa bacteriocins. The Man-PTS is a major sugar uptake system found in many bacteria. The bacteriocins are thought to block open the Man-PTS, allowing free-flow of ions across the membrane, which ultimately leads to cell death (Kjos et al., 2011, Appl. Environ. Microbiol. 77:3335-42). Some of the major proposed resistance mechanisms include down-regulation of the Man-PTS, alterations to membrane composition and charge, and random mutations in the Man-PTS locus (Kjos et al., 2011, Appl. Environ. Microbiol. 77:3335-4220, Opsata et al., 2010, BMC Microbiol. 10:224, Vadyvaloo et al., 2002, Appl. Environ. Microbiol. 68:5223-5230). These mechanisms have been found to differ among species as well among mutants of the same species found to have varying levels of resistance (Opsata et al., 2010, BMC Microbiol. 10:224).

Due to the rapid development of resistance to all three class IIa bacteriocins and the high fraction of resistant mutants in the unexposed culture (˜1 mutant/50,000 bacteria), it is tempting to propose that the primary mode of resistance observed in this study relies on the down-regulation of the Man-PTS. As previously discussed by Kjos and co-workers, it is possible that this down-regulation could be the result of randomness in E. faecium's metabolic gene regulation—a survival tactic referred to as metabolic variability (Kjos et al., 2011, Appl. Environ. Microbiol. 77:3335-42). This hypothesis is further supported by a transcriptome analysis on Pediocin-resistant E. faecalis mutants that found mutants had altered transcription of approximately 200 genes, most of which related to metabolism and transport (Opsata et al., 2010, BMC Microbiol. 10:224). It is possible that E. faecium has a subpopulation that has switched their metabolism to paths not requiring the Man-PTS, relying on alternative carbon sources. At this point, this is only speculation and further, more extensive studies will be needed to determine the true cause of resistance.

As an extension of the delivery system developed in this study, we explored the combination of bacterial AMP delivery with traditional antibiotic therapies to help improve our current system by reducing the rise of resistant mutants (Lewis, 2013, Nat. Rev. Drug Discov. 12:371-87). This-type of combination therapy is conceptually similar to adding alternative AMPs with orthogonal targets to those of the bacteriocins currently in our system. These combination studies successfully showed that the application of 30 μg/mL of rifampicin held off resistant mutants for over 24 hours when combined with the three AMPs. These results are interesting because VRE are often considered inherently resistant to rifampicin. It is possible that the AMPs help permeabilize the cell membrane as previous studies have found that cell membrane permeability likely plays a major role in bacterial susceptibility to rifampicin (Abadi et al., 1996, Antimicrob. Agents Chemother. 40:646-51). The reduction of bacterial resistance is essential and must be carefully considered for both traditional and new antibiotic technologies.

### Concluding Remarks

With this study, we have identified and implemented a chloride-inducible promoter for the production of AMPs. This expression system shows promise for the production of a broad range of AMPs in GI tract environments without the need for added induction molecules. As an example of the application of the chloride-inducible promoter, we showed that the expression of AMPs under the new expression system drastically decreases counts of E. faecium. Furthermore, we showed that by combining the antibiotic rifampicin with three AMPs produced from this system, the inhibition E. faecium is longer-lasting, with limited re-growth of resistant mutants. This study gives promise that the chloride-inducible promoter can be used as a general expression system for the delivery of a wide array of AMPs targeting different pathogens.

### EXAMPLE 2

### Animal Model Experiments

Murine infection models were used to determine whether treatment with Lactococcus, Lactobacillus, or E. coli Nissle 1917 bacteria, engineered to express and secrete antimicrobial peptides, can reduce or eliminate enterococcal colonization of the gastrointestinal tract of animal hosts.

C57BL/6J or BALB/cJ mice were purchased from Jackson Laboratories. Enterococcal colonization was established by Enterococcus administration in the drinking water or via oral gavage. Treatment groups were administered Lactococcus, Lactobacillus, or E. coli Nissle 1917 probiotic bacterial strains in the drinking water or by oral gavage. Throughout the experiment, mouse feces were sampled for enumeration of both the administered Enterococcus strain as well as the probiotic bacteria. Colony forming units of Enterococcus and the probiotic per gram feces were determined by serial dilution of the fecal matter onto selective growth medium. All experiments were followed by euthanasia to harvest intestinal tissue and contents for bacterial enumeration and/or histological studies. Animals were handled in a University-run barrier facility. The facility includes a Biosafety Level 2 (BSL-2) biohazard area where studies of Enterococcus-infected mice were performed.

All animal use protocols have been approved by the University of Minnesota IACUC and by the Wisconsin Medical College IACUC. For these experiments, we collaborate with Professor

Nita Saltzman at Wisconsin Medical College, who is an expert on colonization of mouse GI tracts with Enterococcus (Kommineni S, Bretl D J, Lam V, Chakraborty R, Hayward M, Simpson P, Cao Y, Bousounis P, Kristich C J, Salzman N H Nature. 2015 Oct 29;526(7575):719-22.)

### Results

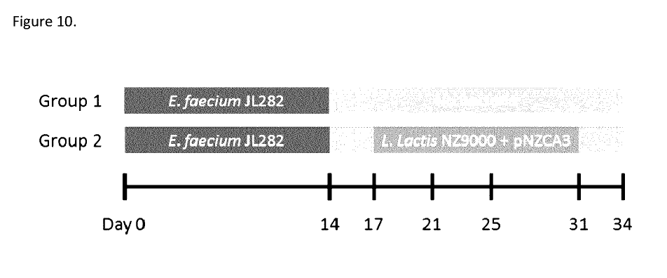

Two groups of 5 mice each were colonized with Enterococcus faecium JL282 (EF). This is a derivative of the clinical isolate strain E. faecium Com12 (Kristich C J and Little J L. Antimicrob Agents Chemother. 2012 Apr;56(4):2022-7). The mice were colonized by oral delivery of 10e8 CFU/ml EF in the drinking water for two weeks. Three days after return to sterile drinking water, when consistent colonization was established, the first group of mice was treated with sterile water, and the second group of mice with water containing 10e8 CFU/ml Lactococcus lactis NZ9000R+PNZCA3 (see Example 1). This is the rifampicin-resistant L. lactis strain equipped with the chloride promoter to produce and secrete bacteriocins Enterocin A, Hiracin JM79, and Enterocin P. This treatment was continued for two more weeks (days 17-31 post infection), after which both groups were returned to sterile water. The experimental timeline is shown in FIG. 10.

E. faecium JL282 and L. lactis pNZCA3 were enumerated in the feces on days 0, 14, 17, 21, 25, 31, and 34. To enumerate colony forming unit (CFU) counts per gram of fecal samples, the samples were plated on Difco m-Enterococcus agar supplemented with 200 μg/ml rifampicin for E. faecium JL282 counts, and on gM17 agar supplemented with 100 μg/ml rifampicin, 20 μg/ml colistin sulfate, 30 μg/ml nalidixic acid and 5 μg/ml chloramphenicol. Day 0 samples were taken to verify that no background growth would be observed on the selective growth media.

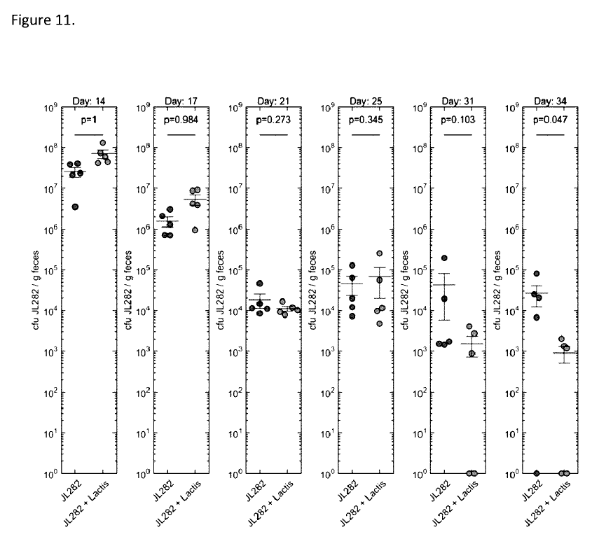

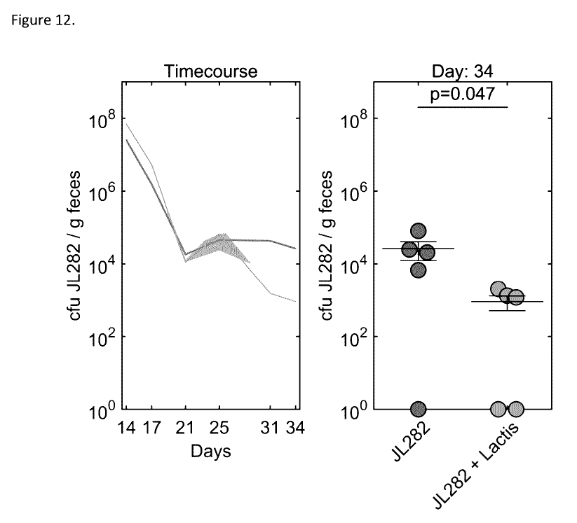

FIG. 11 shows the E. faecium counts for both groups of mice, for all six time points. Error bars presented are standard errors. The p-values are right-tailed Wilcoxon Rank-Sum tests (non parametric tests with no assumptions about the underlying probability distribution of the data). For clarity, the results are shown again, albeit depicted in a different way, in FIG. 12. The left panel in FIG. 12 shows the time-course of the average CFU/g for each group of mice (solid line). The standard errors are depicted continuously as background. On the right, the last time point on day 34 is depicted. The engineered therapeutic strains reduce colonization by JL282 in the mouse gut on day 34.

### EXAMPLE 3

### Combing Enterocin B with Class IIa Bacteriocins to Reduce Resistance Development

Enterocin B is a 53 amino acid bacteriocin naturally produced by Enterococcus faecium and is known to be active against several species of Gram positive bacteria (Casaus et al., 1997, Microbiology 143 (Pt 7):2287-94). Enterocin B belongs to the class II bacteriocins but lacks the conserved Prediocin YGNGVXC, where X is any amino acid (SEQ ID NO:41) amino acid motif, excluding it from the class of IIa bacteriocins. This AMP does however share significant similarity to carnobacteriocin A (Casaus et al., 1997, Microbiology 143 (Pt 7):2287-94).

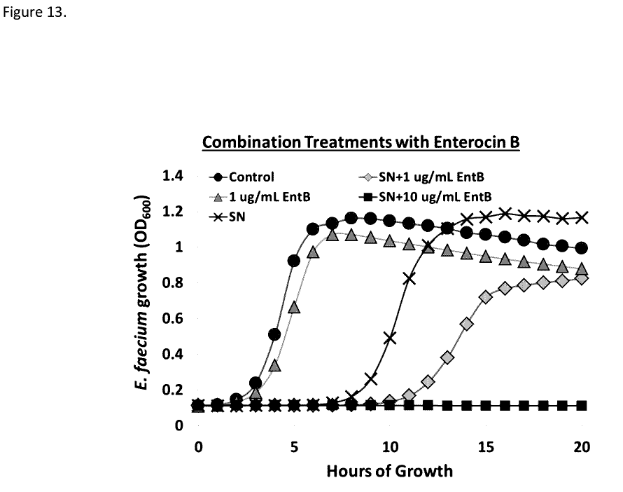

Enterocin B shows significant potential for use in a polycistronic construct with the class IIa bacteriocins Enterocin A, Enterocin P, Hiracin JM79 for the reduction of E. faecium resistance. We have tested the effect of combining pure Enterocin B with L. lactis supernatant containing EntA, EntP, and HirJM79 and found we are able to completely eliminate the rise of resistant E. faecium mutants in an overnight culture. FIG. 13 shows the growth curves of E. faecium 8E9 grown in the absence of any treatment, in the presence of 10% L. lactis supernatant, in the presence of Enterocin B alone, and in the presence of both Enterocin B and supernatant. Based on these results, we anticipate that co-expression of all four peptides will result in a reduced level of pathogen resistance.

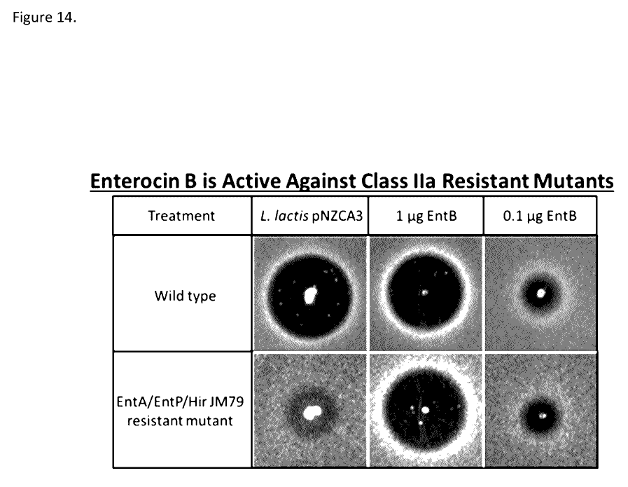

The results demonstrate that this reduction in resistance development is likely due to Enterocin B having an orthogonal mechanism of action to the class IIa bacteriocins. FIG. 14 shows agar diffusion assays of pure Enterocin B against wild type E. faecium 8E9 and an EntA/EntP/HirJM79-resistant mutant of E. faecium 8E9. Notice enterocin B produces similarly sized halos for both the wild type and resistant mutant. Based on the similar halo diameters against the two indicator strains, one can see Enterocin B maintains a similar level of activity against the wild type and class IIa resistant mutants. This indicates that Enterocin B likely acts by a different mechanism of action to EntA/EntP/HirJM79.

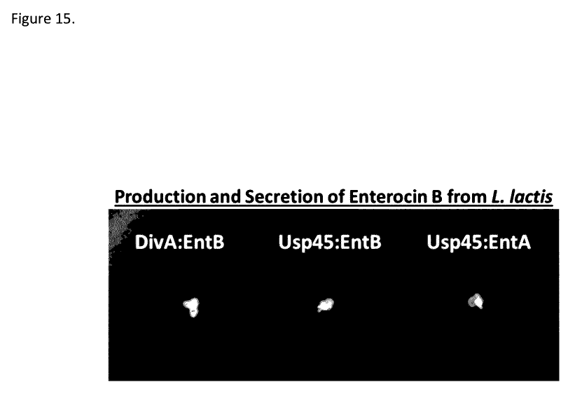

In order to use Enterocin B in a polycistronic construct with other peptides, the production and secretion of the peptides from the same organism was evaluated. Production of Enterocin B from L. lactis was attempted because this organism can also be used for the secretion of EntA, EntP, and HirJM79. We tested both the L. lactis Usp45 signal peptide as well as the Divergicin A signal peptide. FIG. 15 shows the agar diffusion assay of L. lactis producing Enterocin B under the chloride promoter using both signal peptides. The Usp45 signal peptide was selected because we have previously used this tag for the secretion of enterocins from L. lactis (Borrero et al., 2015, ACS Synth. Biol. 4(3):299-306, Geldart et al., 2015, Appl. Environ. Microbiol. 81:3889-3897). The DivA signal peptide was tested as well because this has previously been used with Enterocin B to drive heterologous secretion from E. faecalis (Franz et al., 1999, Appl. Envir. Microbiol. 65:2170-2178). As a comparison, L. lactis producing Enterocin A under the chloride promoter with the Usp45 tag is also shown. These results demonstrate that using the Usp45 secretion tag, we are able to achieve appreciable production and secretion of Enterocin B from the same production system used for the production of EntA, EntP, and HirJM79. It is reasonable to expect that we will be able to add this Usp45:EntB fragment to the pNZCA3 construct and that this addition will reduce the number of E. faecium resistant mutants compared to the original vector.

### Materials and Methods

### Combination Treatment Growth Curves

The Enterocin B peptide (sequence provided below) was synthesized by Ontores. The peptide was received in powder form then resuspended in sterile DI water. Supernatant from L. lactis pNZCA3 containing EntA, EntP, and HirJM79 was produced as follows. An overnight culture of L. lactis grown in BHI was centrifuged at 7,000 rcf for 7 minutes to pellet cells. Supernatant was then filter-sterilized using a 22 μm syringe-driven filter. To prepare the cultures, 5 mL BHI was inoculated with 25 μL E. faecium 8E9 overnight culture. 270 μL of the inoculated culture was added to a well in a 96 well plate. 30 μL of sterile deionized water was added to control cultures, 30 μL of sterile L. lactis supernatant was added for for supernatant (SN) cultures, and concentrated Enterocin B was added to EntB cultures to bring the final concentration to that shown in FIG. 13. The OD600 readings were obtained using a Synergy H1 Plate Reader. During this time, the cultures were incubated at 37° C. After 24 hours, the plate was moved to a 37° C. incubator and incubated for an additional 48 hours to determine if the SN+10 ug/mL Ent B cultures would ever grow and they did not.

### Agar Diffusion Tests

For the agar diffusion activity assays, liquid brain heart infusion (BHI) agar was inoculated with 0.5 μL of E. faecium overnight culture per mL medium. The inoculated agar was then poured into a petri dish and allowed to solidify. For all L. lactis halos, a colony of L. lactis was touched with a sterile pipette tip then stabbed into the inoculated agar. For purified EntB halos (FIG. 14 only), 1 μL of the appropriate EntB concentration was dropped onto the inoculated agar and allowed to dry completely. Dry plates were then covered and incubated overnight at 37° C. for imaging the following day.

### Enterocin B Amino Acid Sequence

ENDHRMPNELNRPNNLSKGGAKCGAAIAGGLFGIPKGPLAWAAGLANVYSKCN (SEQ ID NO:42) (Disulfide bridge between amino acids 23 and 52)

### EXAMPLE 4

### pMPES: a Modular Peptide Expression System for the Delivery of Antimicrobial Peptides

In most naturally-occurring bacterial AMP production systems each AMP has its own dedicated secretion machinery, the genes of which are generally located in close proximity to the AMP and immunity genes. These systems commonly include several large proteins. For some embodiments described herein, it is unfeasible to use unique secretion machinery for each AMP expressed. The simultaneous expression multiple AMP secretion systems would likely burden the expressing strain and DNA manipulation would become excessively costly and cumbersome.

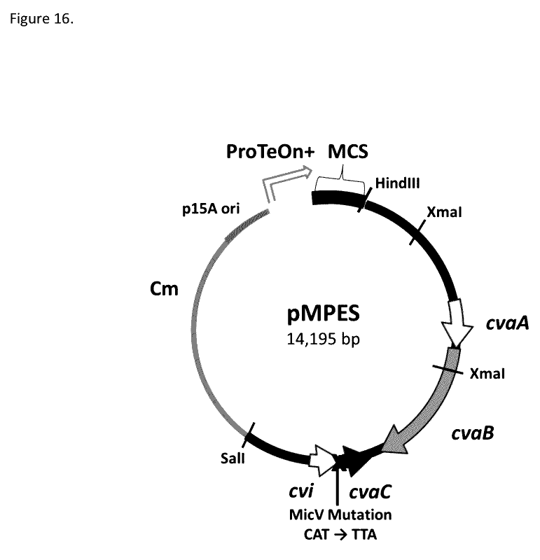

We have created a powerful AMP expression vector, pMPES, which employs an E. coli AMP secretion system to produce and secrete a wide-array of AMPs from probiotic E. coli Nissle 1917. To achieve this, we have remodeled a Microcin V production plasmid, pHK22, so as to create a vector that contains the entire MicV secretion machinery as well as the ProTeOn promoter system and an AMP molecular cloning site. The vector is referred to as pMPES (Modular Peptide Expression System). FIG. 16 shows a diagram of this vector—the components of which are discussed herein. We are able to produce and secrete a variety of diverse AMPs from E. coli Nissle 1917 using this vector. These results are significant because the secretion of different types of peptides from a single organism is a primary limiting factor for the successful use of a polycistronic AMP cassette. To our knowledge, no other vector like this has been created for E. coli and shown to secrete an array of peptides.

The vector pMPES is derived from the plasmid pHK22 which contains a 9.4 kb region encoding the components necessary for Microcin V production and secretion (Gilson et al., 1987, J. Bacteriol. 169:2466-2470), a p15A origin of replication, and a chloramphenicol selection marker. To develop pMPES, we first eliminated native MicV production of pHK22 by mutating the gene's start codon to a stop codon as shown in FIG. 16. This was helpful for the development of an AMP delivery vector because the production of MicV would complicate testing of additional peptides. Elimination of MicV activity was verified using agar diffusion assays against E. coli DH5α. We then cloned the ProTeOn promoter system into the HindIII restriction site shown in FIG. 16 so it was subject to positive feedback under the control of its own promoter. The gene encoding MicV was then inserted downstream of ProTeOn to verify that both the expression and secretion machinery were functional for the native peptide. We then added a carefully designed multiple cloning site and generated a set of corresponding primers that would enable efficient, modular insertion of single or multiple peptides. This component was added to streamline testing of sets of AMPs to identify those with potential for resistance reduction.

It should be noted that previous work has been done to secrete the class IIc AMP Divergicin A from E. coli using the MicV secretion machinery (Van Belkum et al., 1997, Mol. Microbiol. 23:1293-1301). However, this is by no means a guarantee of successful secretion of other peptides using this machinery. AMPS may interact with the secretion tag in ways that inhibit the processing of the tag by the secretion machinery. The tag-AMP molecule may fold so that the tag is physically covered by the AMP, or somehow becomes inaccessible to the secretion machinery. To determine whether this system could be used to secrete a wide array of peptides, we chose three representative class II AMPS to test with the pMPES secretion system: Microcin L, Microcin N, and Enterocin A. Both Microcin L and Microcin N are natively produced by E. coli while Enterocin A is produced by the Gram-positive bacteria Enterococcus faecium. Microcin L was selected for these studies because it is highly homologous to MicV making it a likely successful candidate. Microcin N was selected because though it is an E. coli-derived class II AMP like MicV, it lacks homology with MicV making it representative of a broader group of E. coli-derived AMPs. Lastly, Enterocin A was chosen to test the ability to secrete class II AMPs derived from a variety of bacterial species. In all cases, production was tested for each peptide using both their native signal peptides as well as the MicV signal peptide.

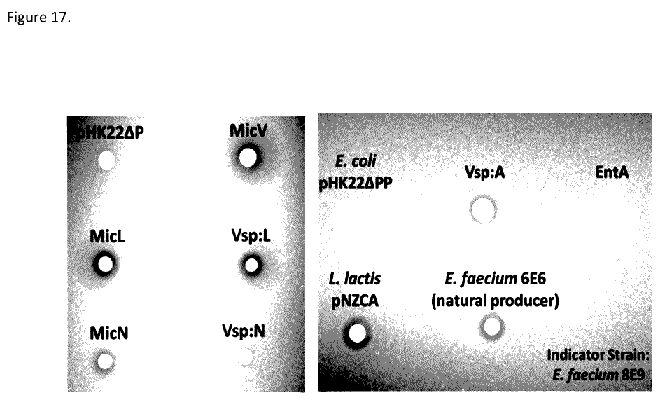

Agar diffusion tests using E. coli DH5α as the indicator strain were used to test for the production of Microcin V, L, and N from pMPES using both their native signal peptides and the MicV signal peptide. FIG. 17, left panel, shows the results of tests which clearly demonstrate the successful production of all three individual peptides from the MicV secretion machinery. Similarly, FIG. 17, right panel, shows the agar diffusion tests showing the production of EntA from pMPES using the native signal peptide and the MicV signal peptide. In these tests, E. faecium 8E9 was used as the indicator strain. As a comparison, this figure also shows EntA production from L. lactis expressing EntA under the chloride-inducible promoter and E. faecium 6E6, a native EntA producer strain. Collectively, these results provide evidence that pMPES can in fact be used to produce and secrete a variety of AMPs derived from both Gram positive and Gram negative bacteria. These results also demonstrate that both the native signal peptide as well as the microcin V signal peptide should be tested when attempting to secrete new AMPs from the system.

We are assembling pMPES with both MicL and Vsp: EntA to show that pMPES can in fact be used to simultaneously produce multiple AMPs in a polycistronic construct. The simultaneous production and secretion of Microcin L and Enterocin A is tested using two agar diffusion assays; one in which E. coli DH5α is used as the indicator strain to test MicL production and one in which E. faecium 8E9 is used as the indicator strain to test EntA production. Based on the results from the individual peptide expression, we anticipate that pMPES can be used to simultaneously produce and secrete multiple AMPs, including Enterocin P, Hiracin JM79, and Enterocin B. Collectively, we have demonstrated that pMPES is a promising and valuable tool that can potentially be used to produce sets of AMPs with reduced bacterial resistance targeting both gram positive and gram negative bacteria.

### Materials and Methods

Construction of pMPES and AMP Insertions

The vector pHK22, originally developed by Gilson et al. (Gilson et al., 1987, J. Bacteriol. 169:2466-2470) was used as the backbone in the creation of pMPES. The full sequence of pMPES is provided below. Throughout this section, column or gel purification of digested vector backbone was performed using the Quiagen QIAquick PCR Purification kit or Gel Extraction Kit respectively. Column purification of all PCR-amplified inserts and insert digests was done using the Quiagen Minelute DNA purification kit. Aside from colony PCRs, all PCRs used NEB Phusion® High-Fidelity DNA Polymerase. Colony PCRs used Promega GoTaq® Green Master Mix. Ligations were done using NEB T4 DNA ligase and assemblies were done using NEBuilder® HiFi DNA Assembly Master Mix. Electrocompetent E. coli MC1061 F′ from Lucigen were used in all transformations unless otherwise stated. All restriction enzymes were purchased from NEB. All procedures were done according to the manufacturer's protocol unless stated otherwise.

First, the start codon of the cvaC gene (sequence provided below) in pHK22 was mutated from ATG to TAA. Note the gene is encoded on the reverse strand such that the mutation was CAT to TTA in the vector map. Mutation was introduced in a piecewise fashion via PCR. First, two fragments were amplified from pHK22 so as to introduce the mutation at their overlap. The fragments were then fused and reinserted into pHK22. Fragment A (˜1.9 kb) which sits between the BssHII restriction site and the cvaC start codon, was generated by PCR using forward primer SDML F (5′-CTGCGCGCATGGTCTTC, SEQ ID NO:43) and reverse primer SDM R (5′-GATAAAAAGGAGATCATTAAAGAACTCTGAC TCTA, SEQ ID NO:44). The primer-introduced mutation is underlined. Fragment B (˜0.5 kb) which sits between the cvaC start codon and the BglII restriction site was generated by PCR using forward primer SDM F (5′-TAGAGTCAGAGTTCTTTAATGATCT CCTTTTTATC, SEQ ID NO:45) and reverse primer SDML R (5′-TTGAGATCTGTTGAG AGGGGTTTT, SEQ ID NO:46). Purified Fragments A and B were then fused using a PCR reaction with primers SDML F and SDML R to give Fragment C (˜2.4 kb). Purified Fragment C and pHK22 were then separately digested with BssHII and BglII by first digesting at 37° C. for 2.5 hours then at 50° C. for another 2.5 hours followed by a 20 minute 65° C. denaturation step. Fragment C digest was column purified and pHK22 was gel purified to isolate the ˜10.5 bp fragment generated. The pHK22 backbone and Fragment C were then ligated using T4 DNA ligase to form pHK22Δ and the resulting ligation was transformed into Lucigen electrocompetent E. coli MC 1061 F′. Successful transformants were first screened using colony PCR with primers SDM_seq_F (5′-CAGCAGCTGCTCCAAT, SEQ ID NO:47) and SDM_seq_R (5′-ATTGCATTAGCTATATATGATTGTGT, SEQ ID NO:48). Correct mutation was then verified with Sanger sequencing. The resulting plasmid is designated pHK22Δ.

For the insertion of the ProTeOn promoter into pHK22Δ, ProTeOn was first amplified using the forward primer Proteon_Assembler_F (5′-GACAGCTTATCATCGATACTAGATAGATGACCTCGGG, SEQ ID NO:49) and the reverse primer Proteon_Assembler_R (5′-CTGGCATTATGGTGGAAAGCTTCAGTTTAATTAAG TGAGCTCACAGCTGTTTC, SEQ ID NO:50) to give the ProTeOn fragment provided below. The promoter fragment was then column purified. pHK22Δ was digested with HindIII restriction enzyme then column purified. Purified pHK22Δ digest and ProTeOn insert were then fused using the NEB Hifi assembly kit. Successful transformants were first screened using colony PCR with primers pHK22 HindIII Seq F (5′-ATGTAGCACCTGAAGTCAGCCC, SEQ ID NO:51) and pHK22 HindIII Seq R (5′ GGTAATAGCGGTAAAGTGGCTAAACGG, SEQ ID NO:52) and were then verified with Sanger sequencing. The resulting plasmid is designated pHK22ΔPP.

To verify the functionality of the expression and secretion machinery, microcin V was inserted into the SacI-PacI restriction sites downstream of ProTeOn in pHK22ΔPP. To do this, the microcin V and microcin V immunity protein were ordered from Geneart. MicV and MicVi were amplified from Geneart MicV by amplification using the forward primer MicV_SacI_F (5′-TGCTAGAGCTCAAGTCAA GGCCGCATCG, SEQ ID NO:53) and the reverse primer MicV_PacI_R (5′-CATAGTTAATTAATCTACAGTGAG CGGAAGGCC, SEQ ID NO:54) to give the Microcin V DNA fragment provided below. pHK22ΔPP and the column-purified MicV insert were then digested using SacI and PacI restriction enzymes. Both digests were then column purified and then ligated together using T4 DNA ligase. Successful transformants were first screened using colony PCR with primers pHK22 HindIII Seq F/R and were then verified with Sanger sequencing.

To test secretion of MicL, VspL, MicN, VspN, EntA, VspA, six gblocks were ordered from Integrated DNA Technologies (IDT). The sequences of these gblocks are provided below in the AMP Fragments section. With the exception of MicL and VspL, all gblocks were inserted directly into SacI-PacI digested pHK22ΔPP using the NEB Hifi assembly kit. In contrast, MicL and VspL were first amplified from their gblocks using the forward primer AMP F (5′-ATCCGGAGCTCTTAGCA, SEQ ID NO:55) and the reverse primer AMP R (5′-TTGACTTAATTAATCGTAGGGGG, SEQ ID NO:56). The resulting MicL and VspL inserts were then digested using SacI and PacI restriction enzymes, column purified, then ligated into SacI-PacI digested pHK22ΔPP using T4 DNA ligase. In all cases, successful transformants were first screened using colony PCR with primers pHK22 HindIII Seq F/R and were then verified with Sanger sequencing. pHK22ΔPP, pHK22ΔPP:V, pHK22ΔPP:L, pHK22ΔPP:VspL, pHK22ΔPP:N, and pHK22ΔPP:VspA were then transformed into electrocompetent E. coli Nissle and shown to produce and secrete all four AMPs.

Negative controls of MicV, MicL, VspL, MicN, and VspA were generated to verify that secretion was in fact achieved through the Microcin V secretion machinery. To do this, vectors were digested with XmaI restriction enzyme to remove the essential MicV secretion genes cvaA and cvaB (see FIG. 16). Vectors were then reclosed via ligation. The resulting ligation was then transformed into E. coli MC1061 F′. Successful transformants were first screened using colony PCR with primers MicV SeC F SpeI (5′-CATAACTAGTATAGGAGGTTGTTTCGCCAGGAT, SEQ ID NO:57) CvaB seq R (5′ACAATCTGATCACAGGCGGT, SEQ ID NO:58) and were then verified with Sanger sequencing using CvaB seq R.

The gblock encoding EntA was made so as to include the pMPES multiple cloning site, the sequence of which is provided below. The AMP-free pMPES was thus generated by first digesting pHK22ΔPP containing the EntA insert (also referred to as pMPES:A) with SacI to cleave out EntA and the immunity gene. The digestion was then column purified using a Qiaquick column. The purified digestion was re-ligated with T4-DNA ligase. The resulting ligation was then transformed into E. coli MC1061 F′. Successful transformants were first screened using colony PCR with primers pHK22 HindIII Seq F/R and were then verified with Sanger sequencing. As stated above, the resulting plasmid is referred to as pMPES.

### Agar Diffusion Tests to Evaluate AMP Production and Secretion

Activity assays were used to determine which AMPs were successfully produced and secreted from E. coli using the pHK22ΔPP expression and secretion system. Note the pHK22ΔPP backbone is identical to pMPES aside from the sequences between the restriction sites in the molecular cloning site which should have minimal impact on protein production. For the agar diffusion activity assays, liquid brain heart infusion (BHI) agar was inoculated with 0.5 μL of E. coli DH5α or E. faecium 8E9 overnight culture per mL medium. The inoculated agar was then poured into a petri dish and allowed to solidify. 0.5 μL overnight culture of each E. coli strain was then dropped onto the plate with the appropriate indicator strain and allowed to dry completely. E. coli MC1061 F′ containing pHK22ΔPP, pHK22ΔPP:V, pHK22ΔPP:L, pHK22ΔPP:VspL, pHK22ΔPP:N, and pHK22ΔPP:VspN were tested against E. coli DH5α indicator strain because Microcin V, Microcin L, and Microcin N are all active against E. coli DH5α. Similarly, pHK22ΔPP, pHK22ΔPP:A, pHK22ΔPP:VspA were tested against E. faecium 8E9 because Enterocin A is known to be active against that strain. Dry plates were then covered and incubated overnight at 37° C. for imaging the following day.

cvaC in pHK22 (start codon is underlined and in
bold)
(SEQ ID NO: 59)
ttataaacaaacatcactaagattatttggactccaattacacaatcttc
ccgcagcatagttccatgettctgaaggtatcccttcgggtttttgctta
attgttccccctaaaccggatggagacattgcaggattaggtttgtgagt
ggatgcatagtcatatattgcacctccagccacacccccagcagctgctc
caattcctcctgcaacaaattgcccggatagtgttcctatagccatcgca
atatcacgccctgaagcaccaccagaaacagaatctaattcatttagagt
cagagttctcat
CvaA gene
(SEQ ID NO: 60)
atgaaaaataatatggagaattacagatactatcagtcaaaaggactgat
taataaagatcaattaactaaccaagttgcattatattatcaacaacaaa
acaaccttctcagtctgagcggacaaaatgaacaaaatgccctgcagata
accactctggagagtcagattcagactcaggcagcagattttgataatcg
tatctatcagatggaactgcaacgactcgaattgcagaaagaactggtta
acactgatgtggaaggcgaaatcattatccgggcgttgtctgacgggaaa
gttgactccctgagtgtcactgtagggcaaatggtcaataccggagacag
ccttctgcaggttattcctgagaacattgaaaactattatcttattctct
gggtcccgaatgatgctgttccttatatttcggctggtgacaaagtgaat
attcgttatgaagccttcccctcagaaaaatttgggcagttctctgctac
ggttaaaactatatccaggactcctgcgtcaacacaggaaatgttgacct
ataagggagcacctcaaaatacgccgggtgcctctgttccctggtataaa
gtcattgcgacgcctgaaaagcagataatcaggtatgacgaaaaatacct
ccctctggaaaatggaatgaaagccgaaagtacactatttctggaaaaaa
ggcgtatttaccagtggatgctttctcctttctatgacatgaaacacagt
gcaacaggaccgatcaatgactaa
CvaA protein
(SEQ ID NO: 61)
MKNNMENYRYYQSKGLINKDQLTNQVALYYQQQNNLLSLSGQNEQNALQI
TTLESQIQTQAADFDNRIYQMELQRLELQKELVNTDVEGEIIIRALSDGK
VDSLSVTVGQMVNTGDSLLQVIPENIENYYLILWVPNDAVPYISAGDKVN
IRYEAFPSEKFGQFSATVKTISRTPASTQEMLTYKGAPQNTPGASVPWYK
VIATPEKQIIRYDEKYLPLENGMKAESTLFLEKRRIYQWMLSPFYDMKHS
ATGPIND
CvaB gene
(SEQ ID NO: 62)
atgactaacaggaatttcagacaaattataaatctgcttgatttgcgctg
gcaacgtcgtgttccggttattcatcagacagagaccgctgaatgtggac
tggcctgcctagcaatgatatgcggtcattttggtaagaatattgacctg
atatatcttcgccggaagtttaatctctctgcccgtggagcaacccttgc
aggaatcaatggaatagcggagcaactggggatggccacccgggctcttt
cactggagttggatgaacttcgagtcctcaaaacgccgtgtattctccac
tgggatttcagtcacttcgtcgttctggtcagcgtaaagcgtaaccgtta
tgtactgcatgatccggccaggggcataagatatatcagccgggaggaaa
tgagccgatattttacaggcgttgcacttgaggtctggcccggaagtgaa
ttccagtcggaaaccctgcagacccgcataagtatcgttcactgattaac
agtatttacggtattaaaagaacgctggcgaaaattttctgtctgtcagt
tgtaattgaagcaatcaatctgctaatgccggtggggacacagctggtta
tggatcatgctattcctgcgggggacagagggctactgacgctaatttct
gctgctcttatgttttttatattactcaaagctgcaacgagtacgctgcg
cgcatggtcttcactggttatgagcacgctcatcaatgtacagtggcagt
cggggctgttcgatcatcttctcagactaccgctggcgttttttgaacgc
cgaaaattaggtgatatccagtcacgttttgactcccttgacacattgag
ggccacatttaccaccagtgtgatcgggttcataatggacagcattatgg
ttgtcggtgtttgtgtgatgatgctgttatacggaggatatctcacctgg
atagttctctgattaccacaatttacatttttattcgactggtgacatac
ggcaattaccgacagatatcagaagaatgtatgtcagggaggcccgtgcc
gcctcctattttatggaaacattatatggtattgccacggtaaaaatcca
ggggatggtcggaattcggggggcacactggcttaatatgaaaatagatg
cgataaattcgggtattaagctaaccaggatggatttgctcttcggagga
ataaatacctttgttaccgcctgtgatcagattgtaattttatggctggg
agcaggccttgtgatcgataatcagatgacaataggaatgtttgtagcgt
ttagttcttttcgtgggcagttttcggaaagagttgcctctctgaccagt
tttcttcttcagctaagaataatgagtctgcacaatgagcgcattgcaga
tattgcattacatgaaaaggaggaaaagaaacctgaaattgaaatcgttg
ctgatatggggccaatatccctggaaaccaatggtttaagctatcgttat
gacagtcagtcagcaccgatattcagtgctctgagtttatctgtagctcc
gggggaaagtgtggctataactggtgcttccggtgcgggaaaaaccacat
taatgaaagtactatgtggactatttgaacctgatagcgggagggtactg
ataaatggtatagatatacgccaaattggaataaataattatcaccggat
gatagcctgtgttatgcaggatgaccggctattttcaggctcaattcgtg
aaaatatctgtggttttgcagaggaaatggatgaagagtggatggtagaa
tgtgccagagcaagtcatattcatgatgttataatgaatatgccaatggg
atatgaaacattaataggtgaacttggggaaggtctttctggcggtcaaa
aacagcgtatatttattgcacgagccttataccggaaaccaggaatatta
tttatggatgaggcaaccagtgctcttgattcagagagtgaacatttcgt
gaatgttgccataaaaaacatgaatatcaccagggtaattattgcacaca
gagaaacaacgttgagaactgttgatagagttatttctatttaa
CvaB protein
(SEQ ID NO:63)
MTNRNFRQIINLLDLRWQRRVPVIHQTETAECGLACLAMICGHFGKNIDL
IYLRRKFNLSARGATLAGINGIAEQLGMATRALSLELDELRVLKTPCILH
WDFSHFVVLVSVKRNRYVLHDPARGIRYISREEMSRYFTGVALEVWPGSE
FQSETLQTRISLRSLINSIYGIKRTLAKIFCLSVVIEAINLLMPVGTQLV
MDHAIPAGDRGLLTLISAALMFFILLKAATSTLRAWSSLVMSTLINVQWQ
SGLFDHLLRLPLAFFERRKLGDIQSRFDSLDTLRATFTTSVIGFIMDSIM
VVGVCVMMLLYGGYLTWIVLCFTTIYIFIRLVTYGNYRQISEECLVREAR
AASYFMETLYGIATVKIQGMVGIRGAHWLNMKIDAINSGIKLTRMDLLFG
GINTFVTACDQIVILWLGAGLVIDNQMTIGMFVAFSSFRGQFSERVASLT
SFLLQLRIMSLHNERIADIALHEKEEKKPEIEIVADMGPISLETNGLSYR
YDSQSAPIFSALSLSVAPGESVAITGASGAGKTTLMKVLCGLFEPDSGRV
LINGIDIRQIGINNYHRMIACVMQDDRLFSGSIRENICGFAEEMDEEWMV
ECARASHIHDVIMNMPMGYETLIGELGEGLSGGQKQRIFIARALYRKPGI
LFMDEATSALDSESEHFVNVAIKNMNITRVIIAHRETTLRTVDRVISI
ProTeOn Promoter Fragment
(SEQ ID NO: 64)
GACAGCTTATCATCGATACTAGATAGATGACCTCGGGGAGCCCGCCTAAT
GAGCGGGCTTTTTGCGCGCCACTCTATCATTGACTCTATCATTGATAGAG
TACTTAACATAAGCACCTGTAGGATCGTACAGGTTTAGCGAAGAAAATGG
TTTGTTATAGTCGAATAAACCTCGAGTTATCTCGAGTGAGATATTGTTGA
CGCACCAAGGAGGAAGCTTCTATGATGAGCCGTCTGGATAAAAGCAAAGT
GATTAATAGCGCACTGGAACTGCTGAATGAAGTTGGTATTGAAGGTCTGA
CCACCCGTAAACTGGCCCAGAAACTGGGTGTTGAACAGCCGACCCTGTAT
TGGCATGTGAAAAATAAACGTGCACTGCTGGATGCACTGGCCGTTGAAAT
TCTGGCTCGCCATCATGATTATAGCCTGCCTGCAGCAGGCGAAAGCTGGC
AGAGCTTTCTGCGTAATAATGCCATGAGCTTTCGTCGTGCCCTGCTGCGT
TATCGTGATGGTGCAAAAGAACATCTGGGCACCCGTCCGGATGAAAAACA
GTATGATACCGTTGAAACCCAGCTGCGTTTTATGACCGAAAATGGTTTTA
GCCTGCGTGATGGTCTGTATGCAATTAGCGCAGTTAGCCATTTTACCCTG
GGTGCCGTTCTGGAACAGCAGGAACATACCGCAGCACTGACCGATCGTCC
TCCGGCACCGGATGAAAATCTGCCTCCGCTGCTGCGTGAAGCACTGATGA
TTATGGATTCTGATGATGGTGAACAGGCATTTCTGCATGGTCTGGAAAGC
CTGATTCGTGGTTTTGAAGTTCAGCTGACCGCACTGCTGCAGATTGTTGG
TGGTGGTGGTGCACGTACCCAGTATAGCGAAAGCATGGGTGCCCGTACAC
AGTATTCTGAATCTATGGGTGCTCGCACCCAGTATTCAGAAAGTATGGGT
GCAAGAACACAGTATAGCGAGTCTATGGGAGCGCGTACTCAGTATAGTGA
ATCAATGGGAGGTGGTATGCCGAGCCTGGTTGATAATTACCGCAAGATTA
ATATTGCCAATAATAAAAGCAACAACGATCTGACCAAACGTGAAAAAGAA
TGTCTGGCCTGGGCATGTGAAGGTAAAAGCAGCTGGGATATTAGCAAAAT
TCTGGGTTGTAGCGAACGTACCGTGACCTTTCATCTGACCAATGCCCAGA
TGAAACTGAATACCACCAATCGTTGTCAGAGCATTAGCAAAGCAATTCTG
ACCGGTGCCATTGATTGTCCGTATTTCAAAAACTGAGAATTCAAGGAGGA
AACAGCTGTGAGCTCACTTAATTAAACTGAAGCTTTCCACCATAATGCCA
G

### Format Coding of AMP Fragments

Ribosomal Binding Sites: lowercased, underlined
Signal peptide: lowercased, bold
Mature peptide: uppercased, bold
Immunity Gene: uppercased, italicized
Terminator: lowercased, italicized, underlined

AMP Fragments
Microcin V
(SEQ ID NO: 65)
gtcaaggccgcatcgaggaggaaacagctatgagaactctgactctaaat
gaattagattctgtttctggtggtGCTTCAGGGCGTGATATTGCGATGGC
TATAGGAACACTATCCGGGCAATTTGTTGCAGGAGGAATTGGAGCAGCTG
CTGGGGGTGTGGCTGGAGGTGCAATATATGACTATGCATCCACTCACAAA
CCTAATCCTGCAATGTCTCCATCCGGTTTAGGGGGAACAATTAAGCAAAA
ACCCGAAGGGATACCTTCAGAAGCATGGAACTATGCTGCGGGAAGATTGT
GTAATTGGAGTCCAAATAATCTTAGTGATGTTTGTTTATAAgataccagg
aggaaactgctATGGATAGAAAAAGAACAAAATTAGAGTTGTTATTTGCA
TTTATAATAAATGCCACCGCAATATATATTGCATTAGCTATATATGATTG
TGTTTTTAGAGGAAAGGACTTTTTATCCATGCATACATTTTGCTTCTCTG
CATTAATGTCTGCAATATGTTACTTTGTTGGTGATAATTATTATTCAATA
TCCGATAAGATAAAAAGGAGATCATATGAGAACTCTGACTCTAAATGAag
gtccatggtacgtacccatagataggcgccgttatcgactgggcctcatg
ggccttccgctcactg
Microcin L
(SEQ ID NO: 66)
atccggagctcttagcaggaggtgaacatgagagaaataacgttaaatga
aatgaataatgtctctggtgctGGTGATGTCAATTGGGTTGATGTCGGGA
AAACTGTAGCAACAAACGGTGCAGGAGTGATTGGCGGTGCATTCGGAGCT
GGTCTGTGCGGCCCTGTTTGTGCTGGTGCCTTCGCTGTTGGATCTTCTGC
CGCTGTTGCTGCTTTGTATGATGCAGCAGGAAATTCCAACTCAGCGAAAC
AAAAACCAGAAGGACTACCTCCAGAAGCATGGAACTACGCTGAAGGTAGA
ATGTGTAATTGGAGTCCAAATAATCTTAGTGATGTTTGTTTATAAactgg
aggaggtgcacATGAAAACTTGGCAGGTTTTCTTCATCATCCTTCCGATC
TCCATCATTATCTCTCTGATTGTTAAACAGCTTAACAGCTCCAACCTTGT
ACAGAGCGTTGTAAGCGGCATTGCTATCGCACTCATGATCTCCATCTTTT
TCAACCGTGGCAAATGAttgacgggcccatgagcctaggtcagtgcggcc
gctacggacatgtccgtagctagcgaaaaaaaaacccgcccctgacaggg
cgggtttttttttaatttaaccccctacgattaattaagtcaa
Vsp:Microcin L
(SEQ ID NO: 67)
atccggagctcttagcaggaggtgaacatgagaactctgactctaaatga
attagattctgtttctggtggtGGTGATGTCAATTGGGTTGATGTCGGGA
AAACTGTAGCAACAAACGGTGCAGGAGTGATTGGCGGTGCATTCGGAGCT
GGTCTGTGCGGCCCTGTTTGTGCTGGTGCCTTCGCTGTTGGATCTTCTGC
CGCTGTTGCTGCTTTGTATGATGCAGCAGGAAATTCCAACTCAGCGAAAC
AAAAACCAGAAGGACTACCTCCAGAAGCATGGAACTACGCTGAAGGTAGA
ATGTGTAATTGGAGTCCAAATAATCTTAGTGATGTTTGTTTATAAactgg
aggaggtgcacATGAAAACTTGGCAGGTTTTCTTCATCATCCTTCCGATC
TCCATCATTATCTCTCTGATTGTTAAACAGCTTAACAGCTCCAACCTTGT
ACAGAGCGTTGTAAGCGGCATTGCTATCGCACTCATGATCTCCATCTTTT
TCAACCGTGGCAAATGAttgacgggcccatgagcctaggtcagtgcggcc
gctacggacatgtccgtagctagcgaaaaaaaaacccgcccctgacaggg
cgggtttttttttaatttaaccccctacgattaattaagTCAA
Microcin N
(SEQ ID NO: 68)
caaggaggaaacagctgtgagctcttagcaggaggtgttcatgtatatgc
gtgagctcgatcgtgaagaacttaactgcgtgggtggcGCGGGGGACCCA
CTCGCAGATCCTAACAGTCAGATCGTTCGCCAGATCATGTCCAATGCGGC
CTGGGGGGCCGCCTTTGGTGCCAGAGGCGGTTTAGGGGGCATGGCGGTTG
GTGCGGCTGGGGGCGTGACTCAAACAGTATTGCAGGGTGCTGCGGCGCAT
ATGCCGGTCAACGTGCCGATTCCTAAGGTGCCGATGGGTCCTAGCTGGAA
CGGGTCCAAAGGCTAAtcaggaggtgctaATGAGCTTTTTGAACTTTGCC
TTTAGCCCCGTGTTTTTTTCCATCATGGCCTGCTATTTCATCGTATGGCG
TAATAAACGCAATGAATTTGTTTGCAACCGCCTGCTGTCGATTATCATCA
TCTCATTTTTAATCTGCTTCATTTACCCATGGCTTAACTATAAAATTGAA
GTGAAATACTACATCTTTGAACAGTTTTACTTGTTCTGCTTCCTGTCGAG
CCTGGTTGCGGTAGTCATTAACCTGATCGTATACTTCATTCTGTACCGTC
GCTGTATTTGAttgacgggcccatgagcctaggtcagtgcggccgctacg
gacatgtccgtagctagcgaaaaaaaaacccgcccctgacagggcgggtt
tttttttaatttaaccccctacgattaattaaactgaagctttccacca
Vsp:Microcin N
(SEQ ID NO: 69)
caaggaggaaacagctgtgagctcttagcaggaggtgaacatgagaactc
tgactctaaatgaattagattctgtttctggtggtGCGGGGGACCCACTC
GCAGATCCTAACAGTCAGATCGTTCGCCAGATCATGTCCAATGCGGCCTG
GGGGGCCGCCTTTGGTGCCAGAGGCGGTTTAGGGGGCATGGCGGTTGGTG
CGGCTGGGGGCGTGACTCAAACAGTATTGCAGGGTGCTGCGGCGCATATG
CCGGTCAACGTGCCGATTCCTAAGGTGCCGATGGGTCCTAGCTGGAACGG
GTCCAAAGGCTAAtcaggaggtgctaATGAGCTTTTTGAACTTTGCCTTT
AGCCCCGTGTTTTTTTCCATCATGGCCTGCTATTTCATCGTATGGCGTAA
TAAACGCAATGAATTTGTTTGCAACCGCCTGCTGTCGATTATCATCATCT
CATTTTTAATCTGCTTCATTTACCCATGGCTTAACTATAAAATTGAAGTG
AAATACTACATCTTTGAACAGTTTTACTTGTTCTGCTTCCTGTCGAGCCT
GGTTGCGGTAGTCATTAACCTGATCGTATACTTCATTCTGTACCGTCGCT
GTATTTGAttgacgggcccatgagcctaggtcagtgcggccgctacggac
atgtccgtagctagcgaaaaaaaaacccgcccctgacagggcgggttttt
ttttaatttaaccccctacgattaattaaactgaagctttccacca
Enterocin A
(SEQ ID NO: 70)
gagaattcaaggaggaaacagctgtgagctcctgatcatgctcaaggagg
agtcagatgaagcatttgaagatcctgagcattaaggagACCCAACTGAT
ATACGGAGGTACCACTCATAGCGGTAAGTATTACGGAAATGGAGTTTACT
GTACCAAAAATAAATGCACCGTTGATTGGGCTAAAGCGACAACTTGTATC
GCTGGTATGTCTATCGGCGGGTTCTTAGGGGGTGCCATTCCAGGCAAATG
CTAAtcgtaggaggacgtacATGAAGAAAAACGCCAAACAGATTGTTCAC
GAGTTATATAACGACATTAGTATCAGCAAAGACCCGAAGTATTCGGACAT
CCTTGAGGTACTGCAAAAAGTGTATCTGAAACTGGAAAAACAGAAATATG
AGTTAGATCCAGGACCATTGATCAATCGTCTGGTAAACTACCTGTACTTC
ACCGCCTATACCAACAAGATTCGTTTCACCGAATATCAAGAAGAGTTAAT
CCGCAATCTGTCTGAGATTGGACGCACAGCAGGAATCAACGGACTGTACC
GTGCCGACTATGGAGATAAAAGTCAGTTCTAAggtcgaagctcagaggat
cgtacaggagctcccacagtagttacagagggcccctgcagctatagcga
ccctaggcctagaacatatagcggccgcccatcacactgtgagcacatgt
ggacacttcaccacgtatctgggctagcgaaaaaaaaacccgcccctgac
agggcgggtttttttttaatttaaccccctacgattaattaaactgaagc
tttccaccataatgcc
Vsp:Enterocin A
(SEQ ID NO: 71)
gagaattcaaggaggaaacagctgtgagctcttagcaggaggtgaacatg
agaactctgactctaaatgaattagattctgtttctggtggtACCACTCA
TAGCGGTAAGTATTACGGAAATGGAGTTTACTGTACCAAAAATAAATGCA
CCGTTGATTGGGCTAAAGCGACAACTTGTATCGCTGGTATGTCTATCGGC
GGGTTCTTAGGGGGTGCCATTCCAGGCAAATGCTAAtgctaaggaggtgc
taATGAAGAAAAACGCCAAACAGATTGTTCACGAGTTATATAACGACATT
AGTATCAGCAAAGACCCGAAGTATTCGGACATCCTTGAGGTACTGCAAAA
AGTGTATCTGAAACTGGAAAAACAGAAATATGAGTTAGATCCAGGACCAT
TGATCAATCGTCTGGTAAACTACCTGTACTTCACCGCCTATACCAACAAG
ATTCGTTTCACCGAATATCAAGAAGAGTTAATCCGCAATCTGTCTGAGAT
TGGACGCACAGCAGGAATCAACGGACTGTACCGTGCCGACTATGGAGATA
AAAGTCAGTTCTAAttgacgggcccatgagcctaggtcagtgeggccgct
acggacatgtccgtagctagcgaaaaaaaaacccgcccctgacagggcgg
gtttttttttaatttaaccccctacgattaattaaactgaagctttccac
cataatgcc
pMPES Multiple Cloning Site (restriction sites
underlined, terminator in bold)
(SEQ ID NO: 72)
gagaattcaaggaggaaacagctgtgagctcccacagtagttacagaggg
cccctgcagctatagcgaccctaggcctagaacatatagcggccgcccat
cacactgtgagcacatgtggacacttcaccacgtatctgggctagcgaaa
aaaaaacccgcccctgacagggcgggtttttttttaatttaaccccccta
cgattaattaaactgaagctttccaccataatgcc
pMPES Full Sequence
(SEQ ID NO: 73)
CTACATATCCTGGTATTTTTTTCCGATTATCTATAACTTGACGTGCAACG
GAAATTTGCCGTTTAGCCACTTTACCGCTATTACCATGGCTACAATCAAT
TATCAGGCGATGGTTAATGCCCTCATCATGCATTAATTTCACTGCCTTAG
TTATATCAGACAAACCATAGTTAGGTTCTCTACCTCCACGTAATATTAGA
TGTCCATGTAGATTGCCATCGGTTAATAGTGTAGATATACTATTTGTTAA
AGATGTCATATATACAATATGTTGTTCACGGGCTGCAATAATAGCGTCAA
TAGCTAAGTTAATATTTCCATCAGTACTGTTTTTAAATCCAACTGGACAA
TGTAAACCAGACGCAAGTTGACGATGCGTTTGTGATTTCAGTGGTTCTGG
CACCAATAGCACCCCAACATATTAAATCAGCAATATAGGGAGTAAGAAAA
GGATCGAGGAATTCTGTAGCGGTTGCGACACGCATCGTTGTGATTGAAGA
CAAACACTGGCGAGCATATCGTATCCCTTTCTCAACATCATAACTTCCAT
TTAGATCAGGATCGTGCATTATTCCCTTCCAGCCTTTACGGGTTCGAGGT
TTCTCAAAATAAGTGCGCATTACAATGTACATTTTCGATAAGTACTTATT
CTGTAATACGAACAATCGCTTCGCATAATCAACTGCGGCCTGAACGTCAT
GGATTGAGCATGGACCAACAATCACAAGTAAGCGAGGATCTTTTCCCAGC
AAAATATTCGCTATAATATCTCGTTGCAGAGAGATCCATGTTACGGTTTC
TTCCGATACTGCGATTTCTTTATGAATCTCGCCCACAGTGGGTAAACTGC
CAAGCAACTTCCCGCGGTAGATTAATTTACTAGCTGACTGCATCTTGTCT
CCTAAAAGCGCTATTTCAGACCATCAGTTGCACACATTAGATGATAATGA
TAATCATTTACTGTATAATTGGCAATATATGGGGATTCATAGTCATTTCT
GAGATAATATACGTTAGGCCTTCTCTCCCGTACGGAAATCACTTGTTTTC
CGTAAATTACCTGAATCTTAATCCCTGAGCTGCGGCTAGCTGTGGAACTC
ACAAAAGCTGACCGGGCCGTGGCACAGCCTCTCTGCTGGCAGAACAGCGT
ACTGCAGAAGTCGTCGTACTGCCGCTGGTCCCGGCATCAGGTCTGCCCGC
AGATGGCACTTCTGCCACTGATCTGCCGGTGGAGCTGAACGGCATGACAT
ATCAGGCCTGCCGTGGGGATTTTGTGGTGCGCCTCGACGGCAGCACCTGT
CTGCAGTTATAGAATAAAGAAGGCGGGGAGGTACGCAGGGAGGGCGATCC
GCTGGAAGTGGCGTAGTGGCTGTAGGCCTGTAATGATGCAGGAATAGAAG
TTCGTGTACAGGTTAATGAAAGCATCACCCCGTAAAAACATGCCGGCACG
GTTTACACATCATTCCATGGTGGCAGCCGGTTAACTTTCCTGCGCCACCA
GTGGAAAGAGGCCATGAGCAGTCCGAACAAAAAACCACTCGTGATACTTA
TAATAATGGCCTCACCTGCTACCATTCCTGACGGCCCCCAGTACATAAAC
CACATTGCACATCCCCAGGAAATACCCCATAAGCCCCCCATCAGCAGCGT
AACCTGCCAGAACGGCATAAAGGGCAATGGCGGAAGCCGGATACCCAGCC
GCCAGAGAATACGCAGCAGAGGAGGAGCATAATTACTCCGCCACATCTTT
TTGCTGTCCATCAGGGCAATTGCCCGGGCTTTTTTTGCTCAAAAGTCACA
GGAGCACCTCCGTTCCATGATGTAAACGTGACACAACTTATTGTTATTGA
TTATTATTATCATACCGTGATTATGTTGTCATGCACCTGTACACTGATAA
AAGAAAGGGAGAACAGGAGGCTGGCTGAACAACCAATCCGTTCGTCCAGT
TCAATGGCGGCTATCAGGTTCTGCTCCGCAATGTACTCACGAATGCGCTT
CCTGTCCTGGCTGGCCAGCATGGTCCAGAATATCTCCATCATTTACCGGC
GACTTTCCTGCTCATTTCATCCCGCCATGCAGCAGCGGAAGATTCGACCA
TGTCATGGGGAAGCATATCGCCGCGCTCGAGTTGCTGACGTCCTGCCTCA
ACCTGGTCACGGAACCAGGTATTATGCTGCCGTTCCACGGTCTGGCGCAT
AAAATCCCGGATTAACTGAGAGCCATTACGGTCCATGCTTTTTGCAGGGC
CATAAAAGCATCCTTCAGTTCAGCGTCAATTCTTAAACTCATATTAACCT
GTGCCATTTATCACCCCGTCATTACCACGTATATACACAGTATATAACGA
TCAGATATCGTCACTAGGATATGCCGCGCCAGCGGCATGGAAGGCGGCAC
TCCGCTGTTTCATATGATACCGCCGAAGCCCGATGTAAGCCGCTACAGTC
GTCCGAAAGTCACCAGCCTCCTCCCCCCTGCCGTCATCCGTGCATCAGAT
ATGCACTGAGTATGCCTGCCCTTCCCTAGAGAATCCTGCCAGGCTTGCCA
CACTGATATATCTTGACTTTATGTAAACGATATGACACTTTAACATGATA
ATGATTACCATTCTCTTTTAATATACAGAGAAACTAGGAAATAGATGAAT
GAGTTATGTTACTTTAATATTCTCTGACAATAACCTAAATCAGTTAGATT
ATTGTCATTTAATAAATAATGACATTCTTTCATCATAAATAAAAAGACTA
TTGTTTATAATATTGTTCTCAGCATTATATGATTATTTATCCTGATAACT
CTCCTATGTTGTATGTTTATATGATTTTCCTTGAAACATATAATGCAAAT
TTTCGATTTATTTTCCATCATTAATCCAGATAAACAACAAACTAATAGTA
TGCAAGGAGACATTATTTGTTTCGCCAGGATGCTTTAGAAAACAGAAAAA
TGAAGTGGCAGGGACGGGCAATATTACTTCCCGGAATACCACTGTGGTTA
ATCATGCTGGGAAGCATTGTGTTTATTACGGCATTTCTGATGTTCATTAT
TGTTGGTACCTATAGCCGCCGTGTTAATGTCAGTGGTGAGGTCACACCTG
GCCAAGAGCTGTCAATATATATTCAGGTGTACAGGGATTTGTTGTCAGGC
AGTTTGTTCATGAAGGGCAGTTGATAAAAAAAGGGGATCCTGTTTATCTG
ATTGACATCAGTAAAAGTACACGCAATGGTATTGTCACTGATAATCATCG
CCGGGATATAGAAAACCAGCTGGTTCGTGTGGACAACATTATTTCCCGTC
TGGAAGAAAGTAAAAAAATAACGCTAGATACCCTGGAAAAACAACGTCTG
CAATACACAGATGCGTTCCGTCGCTCATCAGACATTATACAGCGTGCAGA
GGAAGGGATAAAAATAATGAAAAATAATATGGAGAATTACAGATACTATC
AGTCAAAAGGACTGATTAATAAAGATCAATTAACTAACCAAGTTGCATTA
TATTATCAACAACAAAACAACCTTCTCAGTCTGAGCGGACAAAATGAACA
AAATGCCCTGCAGATAACCACTCTGGAGAGTCAGATTCAGACTCAGGCAG
CAGATTTTGATAATCGTATCTATCAGATGGAACTGCAACGACTCGAATTG
CAGAAAGAACTGGTTAACACTGATGTGGAAGGCGAAATCATTATCCGGGC
GTTGTCTGACGGGAAAGTTGACTCCCTGAGTGTCACTGTAGGGCAAATGG
TCAATACCGGAGACAGCCTTCTGCAGGTTATTCCTGAGAACATTGAAAAC
TATTATCTTATTCTCTGGGTCCCGAATGATGCTGTTCCTTATATTTCGGC
TGGTGACAAAGTGAATATTCGTTATGAAGCCTTCCCCTCAGAAAAATTTG
GGCAGTTCTCTGCTACGGTTAAAACTATATCCAGGACTCCTGCGTCAACA
CAGGAAATGTTGACCTATAAGGGAGCACCTCAAAATACGCCGGGTGCCTC
TGTTCCCTGGTATAAAGTCATTGCGACGCCTGAAAAGCAGATAATCAGGT
ATGACGAAAAATACCTCCCTCTGGAAAATGGAATGAAAGCCGAAAGTACA
CTATTTCTGGAAAAAAGGCGTATTTACCAGTGGATGCTTTCTCCTTTCTA
TGACATGAAACACAGTGCAACAGGACCGATCAATGACTAACAGGAATTTC
AGACAAATTATAAATCTGCTTGATTTGCGCTGGCAACGTCGTGTTCCGGT
TATTCATCAGACAGAGACCGCTGAATGTGGACTGGCCTGCCTAGCAATGA
TATGCGGTCATTTTGGTAAGAATATTGACCTGATATATCTTCGCCGGAAG
TTTAATCTCTCTGCCCGTGGAGCAACCCTTGCAGGAATCAATGGAATAGC
GGAGCAACTGGGGATGGCCACCCGGGCTCTTTCACTGGAGTTGGATGAAC
TTCGAGTCCTCAAAACGCCGTGTATTCTCCACTGGGATTTCAGTCACTTC
GTCGTTCTGGTCAGCGTAAAGCGTAACCGTTATGTACTGCATGATCCGGC
CAGGGGCATAAGATATATCAGCCGGGAGGAAATGAGCCGATATTTTACAG
GCGTTGCACTTGAGGTCTGGCCCGGAAGTGAATTCCAGTCGGAAACCCTG
CAGACCCGCATAAGTCTTCGTTCACTGATTAACAGTATTTACGGTATTAA
AAGAACGCTGGCGAAAATTTTCTGTCTGTCAGTTGTAATTGAAGCAATCA
ATCTGCTAATGCCGGTGGGGACACAGCTGGTTATGGATCATGCTATTCCT
GCGGGGGACAGAGGGCTACTGACGCTAATTTCTGCTGCTCTTATGTTTTT
TATATTACTCAAAGCTGCAACGAGTACGCTGCGCGCATGGTCTTCACTGG
TTATGAGCACGCTCATCAATGTACAGTGGCAGTCGGGGCTGTTCGATCAT
CTTCTCAGACTACCGCTGGCGTTTTTTGAACGCCGAAAATTAGGTGATAT
CCAGTCACGTTTTGACTCCCTTGACACATTGAGGGCCACATTTACCACCA
GTGTGATCGGGTTCATAATGGACAGCATTATGGTTGTCGGTGTTTGTGTG
ATGATGCTGTTATACGGAGGATATCTCACCTGGATAGTTCTCTGCTTTAC
CACAATTTACATTTTTATTCGACTGGTGACATACGGCAATTACCGACAGA
TATCAGAAGAATGTCTTGTCAGGGAGGCCCGTGCCGCCTCCTATTTTATG
GAAACATTATATGGTATTGCCACGGTAAAAATCCAGGGGATGGTCGGAAT
TCGGGGGGCACACTGGCTTAATATGAAAATAGATGCGATAAATTCGGGTA
TTAAGCTAACCAGGATGGATTTGCTCTTCGGAGGAATAAATACCTTTGTT
ACCGCCTGTGATCAGATTGTAATTTTATGGCTGGGAGCAGGCCTTGTGAT
CGATAATCAGATGACAATAGGAATGTTTGTAGCGTTTAGTTCTTTTCGTG
GGCAGTTTTCGGAAAGAGTTGCCTCTCTGACCAGTTTTCTTCTTCAGCTA
AGAATAATGAGTCTGCACAATGAGCGCATTGCAGATATTGCATTACATGA
AAAGGAGGAAAAGAAACCTGAAATTGAAATCGTTGCTGATATGGGGCCAA
TATCCCTGGAAACCAATGGTTTAAGCTATCGTTATGACAGTCAGTCAGCA
CCGATATTCAGTGCTCTGAGTTTATCTGTAGCTCCGGGGGAAAGTGTGGC
TATAACTGGTGCTTCCGGTGCGGGAAAAACCACATTAATGAAAGTACTAT
GTGGACTATTTGAACCTGATAGCGGGAGGGTACTGATAAATGGTATAGAT
ATACGCCAAATTGGAATAAATAATTATCACCGGATGATAGCCTGTGTTAT
GCAGGATGACCGGCTATTTTCAGGCTCAATTCGTGAAAATATCTGTGGTT
TTGCAGAGGAAATGGATGAAGAGTGGATGGTAGAATGTGCCAGAGCAAGT
CATATTCATGATGTTATAATGAATATGCCAATGGGATATGAAACATTAAT
AGGTGAACTTGGGGAAGGTCTTTCTGGCGGTCAAAAACAGCGTATATTTA
TTGCACGAGCCTTATACCGGAAACCAGGAATATTATTTATGGATGAGGCA
ACCAGTGCTCTTGATTCAGAGAGTGAACATTTCGTGAATGTTGCCATAAA
AAACATGAATATCACCAGGGTAATTATTGCACACAGAGAAACAACGTTGA
GAACTGTTGATAGAGTTATTTCTATTTAAACCATAGAGGAATTACAAGCG
TATGAGGAATATTTCTTCCTGTTATAATTCCTCGTTATGCTCAGATATCT
GTTGGAGGTGGAATGGAAGATAGACAATCCACCAAGAAGAAATATCATTC
TGTGTGGATTGTCCAATAACTGTTCTTTCTTATATTAAATAATACTATTT
ATAAACAAACATCACTAAGATTATTTGGACTCCAATTACACAATCTTCCC
GCAGCATAGTTCCATGCTTCTGAAGGTATCCCTTCGGGTTTTTGCTTAAT
TGTTCCCCCTAAACCGGATGGAGACATTGCAGGATTAGGTTTGTGAGTGG
ATGCATAGTCATATATTGCACCTCCAGCCACACCCCCAGCAGCTGCTCCA
ATTCCTCCTGCAACAAATTGCCCGGATAGTGTTCCTATAGCCATCGCAAT
ATCACGCCCTGAAGCACCACCAGAAACAGAATCTAATTCATTTAGAGTCA
GAGTTCTCATATGATCTCCTTTTTATCTTATCGGATATTGAATAATAATT
ATCACCAACAAAGTAACATATTGCAGACATTAATGCAGAGAAGCAAAATG
TATGCATGGATAAAAAGTCCTTTCCTCTAAAAACACAATCATATATAGCT
AATGCAATATATATTGCGGTGGCATTTATTATAAATGCAAATAACAACTC
TAATTTTGTTCTTTTTCTATCCATTACTTTTTATCCCATTACTTTCTATC
CCATTACCACACAAACACTAACGATAATGATTATCGTTAACATAGTCAAG
AGTGAAGGGTAGGAGGCCCTCAACCCCCTATAAGGGGTCCGCTTGGAAAA
CGGATTTCCCCACGTCAAGAGAATTGATTTGAACGAGTGGCTTCGCTACA
AACAGCTCCCGTTTGTGTGTGAAAAACCCCTCTCAACAGATCTCAACTCT
GCACGATATCTACAGGAGATTGCTCCAAAAGGCAGTATCGTGTCTTTTAA
CTACCGATATAACCCCGTCATCGACATGATAATGCGTCTGAGAAATGAGA
AGAAATTAGGTGAACTGCATTTCTTCTCAGCAGAATTTAATAAAAATTCT
GCACTGACCCGCCATCACCTGACCTGGCGAGACTCCGCACAACAGAGCAA
AAGCAGCGGTGCTCTGGGCGATCTATCCTGTCATCTGCTTGATTTATTTT
GTTTTATAGGGGAAAGCCCGGTAGTGGTTCATGGGATCAAAACGGTAAAG
GGGACACGTGGGTACAAAATCAGATGGTCAGGTTGAAGTGGATGATAACG
GCTATGTAATGGGCAGTTCAGAAAAGGGAGCTTATTTCAGAGTTCATGCC
AGTAAATCGGAAACAGATCATAATTTGGGACTGCATATACAGCTTGTATT
TGAAAATGGTGAAATCAGATATTCAACCCACCATGAAAATCGCCTGCTCC
TGATTCTATTTAATGATACGAATACGGAAACAATTGGCTTCGACGCTCTG
AAACGCTTGCCTGATCCACCACGGGAGCTTCCATTCTGGTCTGATTCGTT
TATTCATCTTCATGACGACTGGTGTGCTCTTATTAAATATGGTCACTCGT
CTCCTAAGCTGGCGGACCTGTCATCCGGCCTTCATATACAGGAAATAATT
GAGGCATTCTGATATGGATACCATTCTTTTAACCGGGCTGTTTGCTGCAT
TTTTCACAACGTTTGCGTTCGCTCCCCAGAGCATAAAAACTATCAGGACA
CGCAATACCGAAGGTATCTCCGTTGTTATGTATATCATGTTCCTGACAGG
TGTAATCTCATGGATAGCGTATGGCATTATGCGATCTGATTTTGCCGTAC
TGATCGCCAATATTGTCACACTGTTTCTGGCTGCACCAGTGCTTGTAATA
ACACTTATCAACCGCAGGAAAAAACACGTATGAATATTGCAATCATCGGC
GCGGGTCCTGCCGGCATCATTTCTGCCCGTAATGCGATTAAAGCAGGTCA
CTCCGTGGTTCTGTTTGAGAAGAATACCCGAATTGGGGGGATCTGGAACC
CCTGGAGTGGTGGCGCTTACCGTAATGCCTGTATGCAGAACTCTCGTTAT
ACATTTCATTATACTGGCTTTCCCCCTGGCGATATCGATGAATTTCCAGG
AGTGGAACAGGTATTCAGATATCTTTCGGCCGTGGCCGGAGAGGATGCCC
TCCGTGAGTCGATCCGACTGAATACTGAAGTTGTTTCACTCAGAAAAGAC
GCCGGACACCGGGTGATCCGCTGCGCTTCTGAAGGAAAAGACACGGAAGA
CATTTTTGACCGGGTCATCATTGCAACAGGTGAACTCTGGCAACCCCGCC
GGCCCCCCCTGCCAGGTGAGGAAAACTTCTCCGGAACGTTGATCACGTCG
AGAGATTATCAGGAGCCAGAGGCATTTAAAGGAAAAAATATCCTCATCAT
TGGCGGCGGTGTCAGCGGTGCGGATATTGCCTCAGACCTTGTTCCCTTTG
CCAGAAGCGTAAGTCTGTCCGTCAAAAAGATGGGACTTTATCTGCCGAGA
CAATTCCCGACTGGCCCGAATGACATGATGCACTCCTATCTGGGCAGGTG
TCTGCTGAGCCAAATGAATTATGAAGATTTTATCGGTTATCTCGACACCA
TGATGCCTGACTACATGCAGGCCTACCGAGCATCCAGTCTGTTGCCCGAC
ATGGCGAACAATAACGCGGTGCATGTTAACGAAAAAATCATACCGAATGT
GGCCGCCGGTCTGATAAAAGTTAAACCCCAGGCAGAGCGTTTCACCGGAG
AAGGCGCTATTAAGTTTCGTCGACCGATGCCCTTGAGAGCCTTCAACCCA
GTCAGCTCCTTCCGGTGGGCGCGGGGCATGACTATCGTCGCCGCACTTAT
GACTGTCTTCTTTATCATGCAACTCGTAGGACAGGTGCCGGCAGCGCTCT
GGGTCATTTTCGGCGAGGACCGCTTTCGCTGGAGCGCGACGATGATCGGC
CTGTCGCTTGCGGTATTCGGAATCTTGCACGCCCTCGCTCAAGCCTTCGT
CACTGGTCCCGCCACCAAACGTTTCGGCGAGAAGCAGGCCATTATCGCCG
GCATGGCGGCCGACGCGCTGGGCTACGTCTTGCTGGCGTTCGCGACGCGA
GGCTGGATGGCCTTCCCCATTATGATTCTTCTCGCTTCCGGCGGCATCGG
GATGCCCGCGTTGCAGGCCATGCTGTCCAGGCAGGTAGATGACGACCATC
AGGGACAGCTTCAAGGATCGCTCGCGGCTCTTACCAGCCTAACTTCGATC
ACTGGACCGCTGATCGTCACGGCGATTTATGCCGCCTCGGCGAGCACATG
GAACGGGTTGGCATGGATTGTAGGCGCCGCCCTATACCTTGTCTGCCTCC
CCGCGTTGCGTCGCGGTGCATGGAGCCGGGCCACCTCGACCTGAATGGAA
GCCGGCGGCACCTCGCTAACGGATTCACCACTCCAAGAATTGGAGCCAAT
CAATTCTTGCGGAGAACTGTGAATGCGCAAACCAACCCTTGGCAGAACAT
ATCCATCGCGTCCGCCATCTCCAGCAGCCGCACGCGGCGCATCTCGGGCA
GCGTTGGGTCCTGGCCACGGGTGCGCATGATCGTGCTCCTGTCGTTGAGG
ACCCGGCTAGGCTGGCGGGGTTGCCTTACTGGTTAGCAGAATGAATCACC
GATACGCGAGCGAACGTGAAGCGACTGCTGCTGCAAAACGTCTGCGACCT
GAGCAACAACATGAATGGTCTTCGGTTTCCGTGTTTCGTAAAGTCTGGAA
ACGCGGAAGTCCCCTACGTGCTGCTGAAGTTGCCCGCAACAGAGAGTGGA
ACCAACCGGTGATACCACGATACTATGACTGAGAGTCAACGCCATGAGCG
GCCTCATTTCTTATTCTGAGTTACAACAGTCCGCACCGCTGTCCGGTAGC
TCCTTCCGGTGGGCGCGGGGCATGACTATCGTCGCCGCACTTATGACTGT
CTTCTTTATCATGCAACTCGTAGGACAGGTGCCGGCAGCGCCCAACAGTC
CCCCGGCCACGGGGCCTGCCACCATACCCACGCCGAAACAAGCGCCCTGC
ACCATTATGTTCCGGATCTGCATCGCAGGATGCTGCTGGCTACCCTGTGG
AACACCTACATCTGTATTAACGAAGCGCTAACCGTTTTTATCAGGCTCTG
GGAGGCAGAATAAATGATCATATCGTCAATTATTACCTCCACGGGGAGAG
CCTGAGCAAACTGGCCTCAGGCATTTGAGAAGCACACGGTCACACTGCTT
CCGGTAGTCAATAAACCGGTAAACCAGCAATAGACATAAGCGGCTATTTA
ACGACCCTGCCCTGAACCGACGACCGGGTCGAATTTGCTTTCGAATTTCT
GCCATTCATCCGCTTATTATCACTTATTCAGGCGTAGCACCAGGCGTTTA
AGGGCACCAATAACTGCCTTAAAAAAATTACGCCCCGCCCTGCCACTCAT
CGCAGTACTGTTGTAATTCATTAAGCATTCTGCCGACATGGAAGCCATCA
CAGACGGCATGATGAACCTGAATCGCCAGCGGCATCAGCACCTTGTCGCC
TTGCGTATAATATTTGCCCATGGTGAAAACGGGGGCGAAGAAGTTGTCCA
TATTGGCCACGTTTAAATCAAAACTGGTGAAACTCACCCAGGGATTGGCT
GAGACGAAAAACATATTCTCAATAAACCCTTTAGGGAAATAGGCCAGGTT
TTCACCGTAACACGCCACATCTTGCGAATATATGTGTAGAAACTGCCGGA
AATCGTCGTGGTATTCACTCCAGAGCGATGAAAACGTTTCAGTTTGCTCA
TGGAAAACGGTGTAACAAGGGTGAACACTATCCCATATCACCAGCTCACC
GTCTTTCATTGCCATACGGAATTCCGGATGAGCATTCATCAGGCGGGCAA
GAATGTGAATAAAGGCCGGATAAAACTTGTGCTTATTTTTCTTTACGGTC
TTTAAAAAGGCCGTAATATCCAGCTGAACGGTCTGGTTATAGGTACATTG
AGCAACTGACTGAAATGCCTCAAAATGTTCTTTACGATGCCATTGGGATA
TATCAACGGTGGTATATCCAGTGATTTTTTTCTCCATTTTAGCTTCCTTA
GCTCCTGAAAATCTCGATAACTCAAAAAATACGCCCGGTAGTGATCTTAT
TTCATTATGGTGAAAGTTGGAACCTCTTACGTGCCGATCAACGTCTCATT
TTCGCCAAAAGTTGGCCCAGGGCTTCCCGGTATCAACAGGGACACCAGGA
TTTATTTATTCTGCGAAGTGATCTTCCGTCACAGGTATTTATTCGGCGCA
AAGTGCGTCGGGTGATGCTGCCAACTTACTGATTTAGTGTATGATGGTGT
TTTTGAGGTGCTCCAGTGGCTTCTGTTTCTATCAGCTGTCCCTCCTGTTC
AGCTACTGACGGGGTGGTGCGTAACGGCAAAAGCACCGCCGGACATCAGC
GCTAGCGGAGTGTATACTGGCTTACTATGTTGGCACTGATGAGGGTGTCA
GTGAAGTGCTTCATGTGGCAGGAGAAAAAAGGCTGCACCGGTGCGTCAGC
AGAATATGTGATACAGGATATATTCCGCTTCCTCGCTCACTGACTCGCTA
CGCTCGGTCGTTCGACTGCGGCGAGCGGAAATGGCTTACGAACGGGGCGG
AGATTTCCTGGAAGATGCCAGGAAGATACTTAACAGGGAAGTGAGAGGGC
CGCGGCAAAGCCGTTTTTCCATAGGCTCCGCCCCCCTGACAAGCATCACG
AAATCTGACGCTCAAATCAGTGGTGGCGAAACCCGACAGGACTATAAAGA
TACCAGGCGTTTCCCCCTGGCGGCTCCCTCGTGCGCTCTCCTGTTCCTGC
CTTTCGGTTTACCGGTGTCATTCCGCTGTTATGGCCGCGTTTGTCTCATT
CCACGCCTGACACTCAGTTCCGGGTAGGCAGTTCGCTCCAAGCTGGACTG
TATGCACGAACCCCCCGTTCAGTCCGACCGCTGCGCCTTATCCGGTAACT
ATCGTCTTGAGTCCAACCCGGAAAGACATGCAAAAGCACCACTGGCAGCA
GCCACTGGTAATTGATTTAGAGGAGTTAGTCTTGAAGTCATGCGCCGGTT
AAGGCTAAACTGAAAGGACAAGTTTTGGTGACTGCGCTCCTCCAAGCCAG
TTACCTCGGTTCAAAGAGTTGGTAGCTCAGAGAACCTTCGAAAAACCGCC
CTGCAAGGCGGTTTTTTCGTTTTCAGAGCAAGAGATTACGCGCAGACCAA
AACGATCTCAAGAAGATCATCTTATTAATCAGATAAAATATTTCTAGATT
TCAGTGCAATTTATCTCTTCAAATGTAGCACCTGAAGTCAGCCCCATACG
ATATAAGTTGTAATTCTCATGTTTGACAGCTTATCATCGATACTAGATAG
ATGACCTCGGGGAGCCCGCCTAATGAGCGGGCTTTTTGCGCGCCACTCTA
TCATTGACTCTATCATTGATAGAGTACTTAACATAAGCACCTGTAGGATC
GTACAGGTTTAGCGAAGAAAATGGTTTGTTATAGTCGAATAAACCTCGAG
TTATCTCGAGTGAGATATTGTTGACGCACCAAGGAGGAAGCTTCTATGAT
GAGCCGTCTGGATAAAAGCAAAGTGATTAATAGCGCACTGGAACTGCTGA
ATGAAGTTGGTATTGAAGGTCTGACCACCCGTAAACTGGCCCAGAAACTG
GGTGTTGAACAGCCGACCCTGTATTGGCATGTGAAAAATAAACGTGCACT
GCTGGATGCACTGGCCGTTGAAATTCTGGCTCGCCATCATGATTATAGCC
TGCCTGCAGCAGGCGAAAGCTGGCAGAGCTTTCTGCGTAATAATGCCATG
AGCTTTCGTCGTGCCCTGCTGCGTTATCGTGATGGTGCAAAAGAACATCT
GGGCACCCGTCCGGATGAAAAACAGTATGATACCGTTGAAACCCAGCTGC
GTTTTATGACCGAAAATGGTTTTAGCCTGCGTGATGGTCTGTATGCAATT
AGCGCAGTTAGCCATTTTACCCTGGGTGCCGTTCTGGAACAGCAGGAACA
TACCGCAGCACTGACCGATCGTCCTCCGGCACCGGATGAAAATCTGCCTC
CGCTGCTGCGTGAAGCACTGATGATTATGGATTCTGATGATGGTGAACAG
GCATTTCTGCATGGTCTGGAAAGCCTGATTCGTGGTTTTGAAGTTCAGCT
GACCGCACTGCTGCAGATTGTTGGTGGTGGTGGTGCACGTACCCAGTATA
GCGAAAGCATGGGTGCCCGTACACAGTATTCTGAATCTATGGGTGCTCGC
ACCCAGTATTCAGAAAGTATGGGTGCAAGAACACAGTATAGCGAGTCTAT
GGGAGCGCGTACTCAGTATAGTGAATCAATGGGAGGTGGTATGCCGAGCC
TGGTTGATAATTACCGCAAGATTAATATTGCCAATAATAAAAGCAACAAC
GATCTGACCAAACGTGAAAAAGAATGTCTGGCCTGGGCATGTGAAGGTAA
AAGCAGCTGGGATATTAGCAAAATTCTGGGTTGTAGCGAACGTACCGTGA
CCTTTCATCTGACCAATGCCCAGATGAAACTGAATACCACCAATCGTTGT
CAGAGCATTAGCAAAGCAATTCTGACCGGTGCCATTGATTGTCCGTATTT
CAAAAACTGAGAATTCAAGGAGGAAACAGCTGTGAGCTCCCACAGTAGTT
ACAGAGGGCCCCTGCAGCTATAGCGACCCTAGGCCTAGAACATATAGCGG
CCGCCCATCACACTGTGAGCACATGTGGACACTTCACCACGTATCTGGGC
TAGCGAAAAAAAAACCCGCCCCTGACAGGGCGGGTTTTTTTTTAATTTAA
CCCCCCTACGATTAATTAAACTGAAGCTTTCCACCATAATGCCAG

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A vector comprising a polynucleotide that encodes a polycistronic mRNA operably linked to a heterologous promoter, wherein the polycistronic mRNA comprising three coding regions encoding a first, a second, and a third antimicrobial peptide.

2. The vector of claim 1 wherein the heterologous promoter is a first promoter, wherein expression of the operably linked polynucleotide by the first promoter is controlled by a modulatory protein, wherein the vector further comprises a second promoter operably linked to a fourth coding region, and wherein the fourth coding region encodes the modulator protein.

3. The vector of claim 2 wherein the first promoter is a chloride-inducible promoter, and the modulator protein comprises a GadR protein.

4. The vector of claim 1 wherein the heterologous promoter is regulated by a PROTEON or a PROTEOFF modulatory protein.

5. The vector of claim 4 wherein the polycistronic mRNA includes a fourth coding region encoding the PROTEON or the PROTEOFF modulatory protein, wherein expression of the modulatory protein results in positive feedback and increased expression of the modulatory protein and the first, second and third coding regions.

6. The vector of claim 1 wherein the polycistronic mRNA is at least 3,000 nucleotides in length.

7. The vector of claim 1 further comprising a cvaA coding region and a cvaB coding region, and wherein each antimicrobial peptide comprises a leader sequence that is recognized by the CvaA and CvaB proteins and is exported from an E. coli cell when the vector is present in the E. coli cell.

8. A genetically modified microbe comprising the vector of claim 1.

9. The genetically modified microbe of claim 8 wherein the vector is not integrated into the genomic DNA of the genetically modified microbe.

10. The genetically modified microbe of claim 8 wherein the genetically modified microbe is a gram positive microbe.

11. The genetically modified microbe of claim 10 wherein the genetically modified microbe is a lactic acid bacterium.

12. The genetically modified microbe of claim 11 wherein the lactic acid bacterium is a Lactococcus spp. or a Lactobacillus spp.

13. The genetically modified microbe of claim 8 wherein the genetically modified microbe is a gram negative microbe.

14. The genetically modified microbe of claim 13 wherein the gram negative microbe is E. coli.

15. A method for increasing activity against a microbial pathogen, comprising:
exposing a pathogenic microbe to the genetically modified microbe of claim 8 under conditions suitable for expression of the first, second, and third antimicrobial peptides by the genetically modified microbe, wherein the amount of time for regrowth of the pathogenic microbe is increased compared to the amount of time for regrowth of the pathogenic microbe when exposed to a genetically modified microbe expressing the first, the second, or the third antimicrobial peptide.

16. A method for reducing development of resistance, comprising:
exposing a pathogenic microbe to the genetically modified microbe of claim 8 under conditions suitable for expression of the first, second, and third antimicrobial peptides by the genetically modified microbe, wherein the fraction of the population of the pathogenic microbe with resistance to an administered antimicrobial peptide is decreased compared to the fraction of the population of the pathogenic microbe with resistance to an administered antimicrobial peptide when exposed to a genetically modified microbe expressing the first, the second, or the third antimicrobial peptide.

17. The method of claim 15 wherein the exposing occurs in vitro.

18. The method of claim 15 wherein the pathogenic microbe is a Gram positive microbe.

19. A method for treating a subject having a pathogenic microbe, comprising:
administering to the subject having a pathogenic microbe infection the genetically modified microbe of claim 8.

20. The method of claim 19 wherein the pathogenic microbe comprises a member of the genus Enterococcus, wherein the antimicrobial peptides comprise Enterocin A, Enterocin B, Enterocin P, and Hiracin JM79, and the method further comprises administering a rifamycin to the subject.


SEQUENCE LISTING (73 sequences)

SEQ ID NO: 1 - DNA, 1317 bp, Lactococcus lactis
agatctcaaataaaaagagttggttgagatttcaactagctctttttattttaaattggtagctgagcgttgtataagcttttatgtctttctatatcaacttttaatagaaatataaagtaatataaatgtttttataataaattatgtgagatatatttttttgtccgtactggtatagatttgacgattaagtcttaaataagttataatctcaattgcgtaatttcttaaatacagaaataacaactacattggtagactgattaaaaagtgtacttgatgaactgttataaaccttaaaaaaataaaaataatagtttgggggatgttaaagatgtataaaaaatatggagattgttttaaaaagttgcgaaaccaaaagaatttagggttatcatactttagtaaacttggaatagaccgttcaaatatatctagatttgaacatggaaaatgtatgatgagttttgagcgtatagatttgatgttagaagaaatgcaagttccgttatctgagtacgaattgattgtaaataattttatgccgaatttccaagaattttttatattagaattggaaaaagctgaatttagccaaaatcgagataaaataaaagagttgtattctgaggtcaaagaaacggggaatcatttactgacggttaccgtgaaaacgaagcttgggaatataagtcagacagaagttaaagaaattgaagcttatctttgcaatattgaagagtggggatattttgaacttactttattttattttgtatctgattatctcaatgtcaatcaattagaattgctgctttttaattttgataaaagatgtgaaaattactgtagagtcttaaaatatagaaggagactattgcaaatagcctataaaagtgttgcgatatacgcggctaaaggagaaagaaaaaaagccgaaaatattttagaaatgactaaaaaatatcgaactgtgggagtcgatttatattcagaagtattaagacatcttgctagagctatcattatttttaattttgaaaatgcagagattggggaagaaaaaataaattatgctcttgagattttggaagaatttggaggaaagaagataaaagaattctatcagaataaaatggaaaagtatttgaaaaggtcaatttagtctcttttgagctgttgctttaaagcaacagctcaaaagagattttctttattctagagcatatactagagggtgaagataggttgtctgaagcattataacttgtcttttaaaaaattcaatcataaatataaggaggtatgccatgg

SEQ ID NO: 2 - protein, 276 aa, Lactococcus lactis
MYKKYGDCFKKLRNQKNLGLSYFSKLGIDRSNISRFEHGKCMMSFERIDLMLEEMQVPLSEYELIVNNFMPNFQEFFILELEKAEFSQNRDKIKELYSEVKETGNHLLTVTVKTKLGNISQTEVKEIEAYLCNIEEWGYFELTLFYFVSDYLNVNQLELLLFNFDKRCENYCRVLKYRRRLLQIAYKSVAIYAAKGERKKAENILEMTKKYRTVGVDLYSEVLRHLARAIIIFNFENAEIGEEKINYALEILEEFGGKKIKEFYQNKMEKYLKRSI

SEQ ID NO: 3 - protein, 25 aa, Lactococcus plantarum
GAWKNFWSSLRKGFYDGEAGRAIRR

SEQ ID NO: 4 - protein, 32 aa, Lactococcus plantarum
RRSRKNGIGYAIGYAFGAVERAVLGGSRDYNK

SEQ ID NO: 5 - protein, 33 aa, Lactococcus plantarum
FNRGGYNFGKSVRHVVDAIGSVAGIRGILKSIR

SEQ ID NO: 6 - protein, 34 aa, Lactococcus plantarum
VFHAYSARGVRNNYKSAVGPADWVISAVRGFIHG

SEQ ID NO: 7 - protein, 53 aa, E. faecium
ENDHRMPNELNRPNNLSKGGAKCGAAIAGGLFGIPKGPLAWAAGLANVYSKCN

SEQ ID NO: 8 - protein, 75 aa, E. coli
AGDPLADPNSQIVRQIMSNAAWGAAFGARGGLGGMAVGAAGGVTQTVLQGAAAHMPVNVPIPKVPMGPSWNGSKG

SEQ ID NO: 9 - protein, 90 aa, E. coli
GDVNWVDVGKTVATNGAGVIGGAFGAGLCGPVCAGAFAVGSSAAVAALYDAAGNSNSAKQKPEGLPPEAWNYAEGRMCNWSPNNLSDVCL

SEQ ID NO: 10 - skipped

SEQ ID NO: 11 - protein, 47 aa, E. faecium
TTHSGKYYGNGVYCTKNKCTVDWAKATTCIAGMSIGGFLGGAIPGKC

SEQ ID NO: 12 - protein, 44 aa, Escherichia faecium
ATRSYGNGVYCNNSKCWVNWGEAKENIAGIVISGWASGLAGMGH

SEQ ID NO: 13 - protein, 44 aa, E. hirae
ATYYGNGLYCNKEKCWVDWNQAKGEIGKIIVNGWVNHGPWAPRR

SEQ ID NO: 14 - protein, 18 aa, pig
RGGRLCYCRRRFCVCVGR

SEQ ID NO: 15 - protein, 12 aa, artificial
  peptide analog of pig protegrin-1
LTYCRRRFCVTV

SEQ ID NO: 16 - protein, 19 aa, artificial
  engineered antimicrobial peptide
RPDKPRPYLPRPRPPRPVR

SEQ ID NO: 17 - protein, 23 aa, Alytes obstetricans
GLKDIFKAGLGSLVKGIAAHVAN

SEQ ID NO: 18 - protein, 26 aa, chicken
RVKRVWPLVIRTVIAGYNLYRAIKKK

SEQ ID NO: 19 - protein, 74 aa, Escherichia coli
AGDPLADPNSQIVRQIMSNAAWGPPLVPERFRGMAVGAAGGVTQTVLQGAAAHMPVNVPIPKVPMGPSWNGSKG

SEQ ID NO: 20 - protein, 88 aa, Escherichia coli
ASGRDIAMAIGTLSGQFVAGGIGAAAGGVAGGAIYDYASTHKPNPAMSPSGLGGTIKQKPEGIPSEAWNYAAGRLCNWSPNNLSDVCL

SEQ ID NO: 21 - protein, 11 aa, Lactobacillus acidophilus
NVGVLNPPPLV

SEQ ID NO: 22 - protein, 11 aa, Lactobacillus acidophilus
NVGVLNPPMLV

SEQ ID NO: 23 - protein, 11 aa, Lactobacillus acidophilus
NVGVLLPPPLV

SEQ ID NO: 24 - protein, 11 aa, Lactobacillus acidophilus
NVGVLLPPMLV

SEQ ID NO: 25 - protein, 8 aa, Lactobacillus acidophilus
NPSRQERR

SEQ ID NO: 26 - protein, 5 aa, Lactobacillus acidophilus
PDENK

SEQ ID NO: 27 - protein, 289 aa, Enterococcus faecalis
MAGEVFSSLITSVNPNPMNAGSRNGIPIDTIILHHNATTNKDVAMNTWLLGGGAGTSAHYECTPTEIIGCVGEQYSAFHAGGTGGIDVPKIANPNQRSIGIENVNSSGAPNWSVDPRTITNCARLVADICTRYGIPCDRQHVLGHNEVTATACPGGMDVDEVVRQAQQFMAGGSNNAVKPEPSKPTPSKPSNNKNKEGVATMYCLYERPINSKTGVLEWNGDAWTVMFCNGVNCRRVSHPDEMKVIEDIYRKNNGKDIPFYSQKEWNKNAPWYNRLETVCPVVGITKKS

SEQ ID NO: 28 - protein, 410 aa, Enterobacteria phage F1
MSNINMETAIANMYALKARGITYSMNYSRTGADGTGDCSGTVYDSLRKAGASDAGWVLNTDSMHSWLEKNGFKLIAQNKEWSAKRGDVVIFGKKGASGGSAGHVVIFISSTQIIHCTWKSATANGVYVDNEATTCPYSMGWYVYRLNGGSTPPKPNTKKVKVLKHATNWSPSSKGAKMASFVKGGTFEVKQQRPISYSYSNQEYLIVNKGTVLGWVLSQDIEGGYGSDRVGGSKPKLPAGFTKEEATFINGNAPITTRKNKPSLSSQTATPLYPGQSVRYLGWKSAEGYIWIYATDGRYIPVRPVGKEAWGTFKQDIEGGYGSDRVGGSKPKLPAGFTKEEATFINGNAPITTRKNKPSLSSQTATPLYPGQSVRYLGWKSAEGYIWIYATDGRYIPVRPVGKEAWGTFK

SEQ ID NO: 29 - protein, 328 aa, Enterobacteria phage EFAP-1
MKLKGILLSVVTTFGLLFGATNVQAYEVNNEFNLQPWEGSQQLAYPNKIILHETANPRATGRNEATYMKNNWFNAHTTAIVGDGGIVYKVAPEGNVSWGAGNANPYAPVQIELQHTNDPELFKANYKAYVDYTRDMGKKFGIPMTLDQGGSLWEKGVVSHQWVTDFVWGDHTDPYGYLAKMGISKAQLAHDLANGVSGNTATPTPKPDKPKPTQPSKPSNKKRFNYRVDGLEYVNGMWQIYNEHLGKIDFNWTENGIPVEVVDKVNPATGQPTKDQVLKVGDYFNFQENSTGVVQEQTPYMGYTLSHVQLPNEFIWLFTDSKQALMYQ

SEQ ID NO: 30 - protein, 289 aa, Enterobacteria phage jEF24C; identical to SEQ ID NO: 27

SEQ ID NO: 31 - protein, 237 aa, Enterobacteria phage F168/08
MVKLNDVLSYVNGLVGKGVDADGWYGTQCMDLTVDVMQRFFGWRPYGNAIALVDQPIPAGFQRIRTTSSTQIKAGDVMIWGLGYYAQYGHTHIATEDGRADGTFVSVDQNWINPSLEVGSPAAAIHHNMDGVWGVIRPPYEAESKPKPPAPKPDKPNLGQFKGDDDIMFIYYKKTKQGSTEQWFVIGGKRIYLPTMTYVNEANDLIKRYGGNTNVTTYNYDNFGLAMMEKAYPQVKL

SEQ ID NO: 32 - protein, 15 aa, E. coli
MRTLTLNELDSVSGG

SEQ ID NO: 33 - DNA, 18 bp, Artificial
  primer
cgaattgaaggaaggccg

SEQ ID NO: 34 - DNA, 17 bp, Artificial
  primer
gcagtgaaaggaaggcc

SEQ ID NO: 35 - DNA, 20 bp, Artificial
  primer
gccccgttagttgaagaagg

SEQ ID NO: 36 - DNA, 23 bp, Artificial
  primer
caattgaacgtttcaagccttgg

SEQ ID NO: 37 - DNA, 35 bp, Artificial
  primer
gctaagccatggaagttactgacgtaagattacgg

SEQ ID NO: 38 - DNA, 40 bp, Artificial
  primer
tcgactagtttattattatttttgacaccagaccaactgg

SEQ ID NO: 39 - DNA, 35 bp, Artificial
  primer
cataacatgtctactatgaaaaaaaagattatctc

SEQ ID NO: 40 - DNA, 24 bp, Artificial
  primer
cactagtttatcaaagtcccgacc

SEQ ID NO: 41 - protein, 7 aa, artificial
  conserved Prediocin
  misc_feature (6)..(6): Xaa can be any naturally occurring amino acid
YGNGVXC

SEQ ID NO: 42 - protein, 53 aa, Enterococcus faecium; identical to SEQ ID NO: 7

SEQ ID NO: 43 - DNA, 17 bp, Artificial
  primer
ctgcgcgcatggtcttc

SEQ ID NO: 44 - DNA, 35 bp, Artificial
  primer
gataaaaaggagatcattaaagaactctgactcta

SEQ ID NO: 45 - DNA, 35 bp, Artificial
  primer
tagagtcagagttctttaatgatctcctttttatc

SEQ ID NO: 46 - DNA, 24 bp, Artificial
  primer
ttgagatctgttgagaggggtttt

SEQ ID NO: 47 - DNA, 16 bp, Artificial
  primer
cagcagctgctccaat

SEQ ID NO: 48 - DNA, 26 bp, Artificial
  primer
attgcattagctatatatgattgtgt

SEQ ID NO: 49 - DNA, 37 bp, Artificial
  primer
gacagcttatcatcgatactagatagatgacctcggg

SEQ ID NO: 50 - DNA, 53 bp, Artificial
  primer
ctggcattatggtggaaagcttcagtttaattaagtgagctcacagctgtttc

SEQ ID NO: 51 - DNA, 22 bp, Artificial
  primer
atgtagcacctgaagtcagccc

SEQ ID NO: 52 - DNA, 27 bp, Artificial
  primer
ggtaatagcggtaaagtggctaaacgg

SEQ ID NO: 53 - DNA, 28 bp, Artificial
  primer
tgctagagctcaagtcaaggccgcatcg

SEQ ID NO: 54 - DNA, 33 bp, Artificial
  primer
catagttaattaatctacagtgagcggaaggcc

SEQ ID NO: 55 - DNA, 17 bp, Artificial
  primer
atccggagctcttagca

SEQ ID NO: 56 - DNA, 23 bp, Artificial
  primer
ttgacttaattaatcgtaggggg

SEQ ID NO: 57 - DNA, 33 bp, Artificial
  primer
cataactagtataggaggttgtttcgccaggat

SEQ ID NO: 58 - DNA, 20 bp, Artificial
  primer
acaatctgatcacaggcggt

SEQ ID NO: 59 - DNA, 312 bp, E. coli
ttataaacaaacatcactaagattatttggactccaattacacaatcttcccgcagcatagttccatgcttctgaaggtatcccttcgggtttttgcttaattgttccccctaaaccggatggagacattgcaggattaggtttgtgagtggatgcatagtcatatattgcacctccagccacacccccagcagctgctccaattcctcctgcaacaaattgcccggatagtgttcctatagccatcgcaatatcacgccctgaagcaccaccagaaacagaatctaattcatttagagtcagagttctcat

SEQ ID NO: 60 - DNA, 774 bp, E. coli
atgaaaaataatatggagaattacagatactatcagtcaaaaggactgattaataaagatcaattaactaaccaagttgcattatattatcaacaacaaaacaaccttctcagtctgagcggacaaaatgaacaaaatgccctgcagataaccactctggagagtcagattcagactcaggcagcagattttgataatcgtatctatcagatggaactgcaacgactcgaattgcagaaagaactggttaacactgatgtggaaggcgaaatcattatccgggcgttgtctgacgggaaagttgactccctgagtgtcactgtagggcaaatggtcaataccggagacagccttctgcaggttattcctgagaacattgaaaactattatcttattctctgggtcccgaatgatgctgttccttatatttcggctggtgacaaagtgaatattcgttatgaagccttcccctcagaaaaatttgggcagttctctgctacggttaaaactatatccaggactcctgcgtcaacacaggaaatgttgacctataagggagcacctcaaaatacgccgggtgcctctgttccctggtataaagtcattgcgacgcctgaaaagcagataatcaggtatgacgaaaaatacctccctctggaaaatggaatgaaagccgaaagtacactatttctggaaaaaaggcgtatttaccagtggatgctttctcctttctatgacatgaaacacagtgcaacaggaccgatcaatgactaa

SEQ ID NO: 61 - protein, 257 aa, E. coli
MKNNMENYRYYQSKGLINKDQLTNQVALYYQQQNNLLSLSGQNEQNALQITTLESQIQTQAADFDNRIYQMELQRLELQKELVNTDVEGEIIIRALSDGKVDSLSVTVGQMVNTGDSLLQVIPENIENYYLILWVPNDAVPYISAGDKVNIRYEAFPSEKFGQFSATVKTISRTPASTQEMLTYKGAPQNTPGASVPWYKVIATPEKQIIRYDEKYLPLENGMKAESTLFLEKRRIYQWMLSPFYDMKHSATGPIND

SEQ ID NO: 62 - DNA, 2097 bp, E. coli
atgactaacaggaatttcagacaaattataaatctgcttgatttgcgctggcaacgtcgtgttccggttattcatcagacagagaccgctgaatgtggactggcctgcctagcaatgatatgcggtcattttggtaagaatattgacctgatatatcttcgccggaagtttaatctctctgcccgtggagcaacccttgcaggaatcaatggaatagcggagcaactggggatggccacccgggctctttcactggagttggatgaacttcgagtcctcaaaacgccgtgtattctccactgggatttcagtcacttcgtcgttctggtcagcgtaaagcgtaaccgttatgtactgcatgatccggccaggggcataagatatatcagccgggaggaaatgagccgatattttacaggcgttgcacttgaggtctggcccggaagtgaattccagtcggaaaccctgcagacccgcataagtcttcgttcactgattaacagtatttacggtattaaaagaacgctggcgaaaattttctgtctgtcagttgtaattgaagcaatcaatctgctaatgccggtggggacacagctggttatggatcatgctattcctgcgggggacagagggctactgacgctaatttctgctgctcttatgttttttatattactcaaagctgcaacgagtacgctgcgcgcatggtcttcactggttatgagcacgctcatcaatgtacagtggcagtcggggctgttcgatcatcttctcagactaccgctggcgttttttgaacgccgaaaattaggtgatatccagtcacgttttgactcccttgacacattgagggccacatttaccaccagtgtgatcgggttcataatggacagcattatggttgtcggtgtttgtgtgatgatgctgttatacggaggatatctcacctggatagttctctgctttaccacaatttacatttttattcgactggtgacatacggcaattaccgacagatatcagaagaatgtcttgtcagggaggcccgtgccgcctcctattttatggaaacattatatggtattgccacggtaaaaatccaggggatggtcggaattcggggggcacactggcttaatatgaaaatagatgcgataaattcgggtattaagctaaccaggatggatttgctcttcggaggaataaatacctttgttaccgcctgtgatcagattgtaattttatggctgggagcaggccttgtgatcgataatcagatgacaataggaatgtttgtagcgtttagttcttttcgtgggcagttttcggaaagagttgcctctctgaccagttttcttcttcagctaagaataatgagtctgcacaatgagcgcattgcagatattgcattacatgaaaaggaggaaaagaaacctgaaattgaaatcgttgctgatatggggccaatatccctggaaaccaatggtttaagctatcgttatgacagtcagtcagcaccgatattcagtgctctgagtttatctgtagctccgggggaaagtgtggctataactggtgcttccggtgcgggaaaaaccacattaatgaaagtactatgtggactatttgaacctgatagcgggagggtactgataaatggtatagatatacgccaaattggaataaataattatcaccggatgatagcctgtgttatgcaggatgaccggctattttcaggctcaattcgtgaaaatatctgtggttttgcagaggaaatggatgaagagtggatggtagaatgtgccagagcaagtcatattcatgatgttataatgaatatgccaatgggatatgaaacattaataggtgaacttggggaaggtctttctggcggtcaaaaacagcgtatatttattgcacgagccttataccggaaaccaggaatattatttatggatgaggcaaccagtgctcttgattcagagagtgaacatttcgtgaatgttgccataaaaaacatgaatatcaccagggtaattattgcacacagagaaacaacgttgagaactgttgatagagttatttctatttaa

SEQ ID NO: 63 - protein, 698 aa, E. coli
MTNRNFRQIINLLDLRWQRRVPVIHQTETAECGLACLAMICGHFGKNIDLIYLRRKFNLSARGATLAGINGIAEQLGMATRALSLELDELRVLKTPCILHWDFSHFVVLVSVKRNRYVLHDPARGIRYISREEMSRYFTGVALEVWPGSEFQSETLQTRISLRSLINSIYGIKRTLAKIFCLSVVIEAINLLMPVGTQLVMDHAIPAGDRGLLTLISAALMFFILLKAATSTLRAWSSLVMSTLINVQWQSGLFDHLLRLPLAFFERRKLGDIQSRFDSLDTLRATFTTSVIGFIMDSIMVVGVCVMMLLYGGYLTWIVLCFTTIYIFIRLVTYGNYRQISEECLVREARAASYFMETLYGIATVKIQGMVGIRGAHWLNMKIDAINSGIKLTRMDLLFGGINTFVTACDQIVILWLGAGLVIDNQMTIGMFVAFSSFRGQFSERVASLTSFLLQLRIMSLHNERIADIALHEKEEKKPEIEIVADMGPISLETNGLSYRYDSQSAPIFSALSLSVAPGESVAITGASGAGKTTLMKVLCGLFEPDSGRVLINGIDIRQIGINNYHRMIACVMQDDRLFSGSIRENICGFAEEMDEEWMVECARASHIHDVIMNMPMGYETLIGELGEGLSGGQKQRIFIARALYRKPGILFMDEATSALDSESEHFVNVAIKNMNITRVIIAHRETTLRTVDRVISI

SEQ ID NO: 64 - DNA, 1351 bp, ProTeOn Promoter
gacagcttatcatcgatactagatagatgacctcggggagcccgcctaatgagcgggctttttgcgcgccactctatcattgactctatcattgatagagtacttaacataagcacctgtaggatcgtacaggtttagcgaagaaaatggtttgttatagtcgaataaacctcgagttatctcgagtgagatattgttgacgcaccaaggaggaagcttctatgatgagccgtctggataaaagcaaagtgattaatagcgcactggaactgctgaatgaagttggtattgaaggtctgaccacccgtaaactggcccagaaactgggtgttgaacagccgaccctgtattggcatgtgaaaaataaacgtgcactgctggatgcactggccgttgaaattctggctcgccatcatgattatagcctgcctgcagcaggcgaaagctggcagagctttctgcgtaataatgccatgagctttcgtcgtgccctgctgcgttatcgtgatggtgcaaaagaacatctgggcacccgtccggatgaaaaacagtatgataccgttgaaacccagctgcgttttatgaccgaaaatggttttagcctgcgtgatggtctgtatgcaattagcgcagttagccattttaccctgggtgccgttctggaacagcaggaacataccgcagcactgaccgatcgtcctccggcaccggatgaaaatctgcctccgctgctgcgtgaagcactgatgattatggattctgatgatggtgaacaggcatttctgcatggtctggaaagcctgattcgtggttttgaagttcagctgaccgcactgctgcagattgttggtggtggtggtgcacgtacccagtatagcgaaagcatgggtgcccgtacacagtattctgaatctatgggtgctcgcacccagtattcagaaagtatgggtgcaagaacacagtatagcgagtctatgggagcgcgtactcagtatagtgaatcaatgggaggtggtatgccgagcctggttgataattaccgcaagattaatattgccaataataaaagcaacaacgatctgaccaaacgtgaaaaagaatgtctggcctgggcatgtgaaggtaaaagcagctgggatattagcaaaattctgggttgtagcgaacgtaccgtgacctttcatctgaccaatgcccagatgaaactgaataccaccaatcgttgtcagagcattagcaaagcaattctgaccggtgccattgattgtccgtatttcaaaaactgagaattcaaggaggaaacagctgtgagctcacttaattaaactgaagctttccaccataatgccag

SEQ ID NO: 65 - DNA, 666 bp, artificial
  cassette encoding Microcin V
gtcaaggccgcatcgaggaggaaacagctatgagaactctgactctaaatgaattagattctgtttctggtggtgcttcagggcgtgatattgcgatggctataggaacactatccgggcaatttgttgcaggaggaattggagcagctgctgggggtgtggctggaggtgcaatatatgactatgcatccactcacaaacctaatcctgcaatgtctccatccggtttagggggaacaattaagcaaaaacccgaagggataccttcagaagcatggaactatgctgcgggaagattgtgtaattggagtccaaataatcttagtgatgtttgtttataagataccaggaggaaactgctatggatagaaaaagaacaaaattagagttgttatttgcatttataataaatgccaccgcaatatatattgcattagctatatatgattgtgtttttagaggaaaggactttttatccatgcatacattttgcttctctgcattaatgtctgcaatatgttactttgttggtgataattattattcaatatccgataagataaaaaggagatcatatgagaactctgactctaaatgaaggtccatggtacgtacccatagataggcgccgttatcgactgggcctcatgggccttccgctcactg

SEQ ID NO: 66 - DNA, 644 bp, artificial
  cassette encoding Microcin L
atccggagctcttagcaggaggtgaacatgagagaaataacgttaaatgaaatgaataatgtctctggtgctggtgatgtcaattgggttgatgtcgggaaaactgtagcaacaaacggtgcaggagtgattggcggtgcattcggagctggtctgtgcggccctgtttgtgctggtgccttcgctgttggatcttctgccgctgttgctgctttgtatgatgcagcaggaaattccaactcagcgaaacaaaaaccagaaggactacctccagaagcatggaactacgctgaaggtagaatgtgtaattggagtccaaataatcttagtgatgtttgtttataaactggaggaggtgcacatgaaaacttggcaggttttcttcatcatccttccgatctccatcattatctctctgattgttaaacagcttaacagctccaaccttgtacagagcgttgtaagcggcattgctatcgcactcatgatctccatctttttcaaccgtggcaaatgattgacgggcccatgagcctaggtcagtgcggccgctacggacatgtccgtagctagcgaaaaaaaaacccgcccctgacagggcgggtttttttttaatttaacccccctacgattaattaagtcaa

SEQ ID NO: 67 - DNA, 644 bp, artificial
  cassette encoding Microcin L
atccggagctcttagcaggaggtgaacatgagaactctgactctaaatgaattagattctgtttctggtggtggtgatgtcaattgggttgatgtcgggaaaactgtagcaacaaacggtgcaggagtgattggcggtgcattcggagctggtctgtgcggccctgtttgtgctggtgccttcgctgttggatcttctgccgctgttgctgctttgtatgatgcagcaggaaattccaactcagcgaaacaaaaaccagaaggactacctccagaagcatggaactacgctgaaggtagaatgtgtaattggagtccaaataatcttagtgatgtttgtttataaactggaggaggtgcacatgaaaacttggcaggttttcttcatcatccttccgatctccatcattatctctctgattgttaaacagcttaacagctccaaccttgtacagagcgttgtaagcggcattgctatcgcactcatgatctccatctttttcaaccgtggcaaatgattgacgggcccatgagcctaggtcagtgcggccgctacggacatgtccgtagctagcgaaaaaaaaacccgcccctgacagggcgggtttttttttaatttaacccccctacgattaattaagtcaa

SEQ ID NO: 68 - DNA, 750 bp, artificial
  cassette encoding Microcin N
caaggaggaaacagctgtgagctcttagcaggaggtgttcatgtatatgcgtgagctcgatcgtgaagaacttaactgcgtgggtggcgcgggggacccactcgcagatcctaacagtcagatcgttcgccagatcatgtccaatgcggcctggggggccgcctttggtgccagaggcggtttagggggcatggcggttggtgcggctgggggcgtgactcaaacagtattgcagggtgctgcggcgcatatgccggtcaacgtgccgattcctaaggtgccgatgggtcctagctggaacgggtccaaaggctaatcaggaggtgctaatgagctttttgaactttgcctttagccccgtgtttttttccatcatggcctgctatttcatcgtatggcgtaataaacgcaatgaatttgtttgcaaccgcctgctgtcgattatcatcatctcatttttaatctgcttcatttacccatggcttaactataaaattgaagtgaaatactacatctttgaacagttttacttgttctgcttcctgtcgagcctggttgcggtagtcattaacctgatcgtatacttcattctgtaccgtcgctgtatttgattgacgggcccatgagcctaggtcagtgcggccgctacggacatgtccgtagctagcgaaaaaaaaacccgcccctgacagggcgggtttttttttaatttaacccccctacgattaattaaactgaagctttccacca

SEQ ID NO: 69 - DNA, 747 bp, artificial
  cassette encoding Microcin N
caaggaggaaacagctgtgagctcttagcaggaggtgaacatgagaactctgactctaaatgaattagattctgtttctggtggtgcgggggacccactcgcagatcctaacagtcagatcgttcgccagatcatgtccaatgcggcctggggggccgcctttggtgccagaggcggtttagggggcatggcggttggtgcggctgggggcgtgactcaaacagtattgcagggtgctgcggcgcatatgccggtcaacgtgccgattcctaaggtgccgatgggtcctagctggaacgggtccaaaggctaatcaggaggtgctaatgagctttttgaactttgcctttagccccgtgtttttttccatcatggcctgctatttcatcgtatggcgtaataaacgcaatgaatttgtttgcaaccgcctgctgtcgattatcatcatctcatttttaatctgcttcatttacccatggcttaactataaaattgaagtgaaatactacatctttgaacagttttacttgttctgcttcctgtcgagcctggttgcggtagtcattaacctgatcgtatacttcattctgtaccgtcgctgtatttgattgacgggcccatgagcctaggtcagtgcggccgctacggacatgtccgtagctagcgaaaaaaaaacccgcccctgacagggcgggtttttttttaatttaacccccctacgattaattaaactgaagctttccacca

SEQ ID NO: 70 - DNA, 817 bp, artificial
  cassette encoding Enterocin A
gagaattcaaggaggaaacagctgtgagctcctgatcatgctcaaggaggagtcagatgaagcatttgaagatcctgagcattaaggagacccaactgatatacggaggtaccactcatagcggtaagtattacggaaatggagtttactgtaccaaaaataaatgcaccgttgattgggctaaagcgacaacttgtatcgctggtatgtctatcggcgggttcttagggggtgccattccaggcaaatgctaatcgtaggaggacgtacatgaagaaaaacgccaaacagattgttcacgagttatataacgacattagtatcagcaaagacccgaagtattcggacatccttgaggtactgcaaaaagtgtatctgaaactggaaaaacagaaatatgagttagatccaggaccattgatcaatcgtctggtaaactacctgtacttcaccgcctataccaacaagattcgtttcaccgaatatcaagaagagttaatccgcaatctgtctgagattggacgcacagcaggaatcaacggactgtaccgtgccgactatggagataaaagtcagttctaaggtcgaagctcagaggatcgtacaggagctcccacagtagttacagagggcccctgcagctatagcgaccctaggcctagaacatatagcggccgcccatcacactgtgagcacatgtggacacttcaccacgtatctgggctagcgaaaaaaaaacccgcccctgacagggcgggtttttttttaatttaacccccctacgattaattaaactgaagctttccaccataatgcc

SEQ ID NO: 71 - DNA, 710 bp, artificial
  cassette encoding Enterocin A
gagaattcaaggaggaaacagctgtgagctcttagcaggaggtgaacatgagaactctgactctaaatgaattagattctgtttctggtggtaccactcatagcggtaagtattacggaaatggagtttactgtaccaaaaataaatgcaccgttgattgggctaaagcgacaacttgtatcgctggtatgtctatcggcgggttcttagggggtgccattccaggcaaatgctaatgctaaggaggtgctaatgaagaaaaacgccaaacagattgttcacgagttatataacgacattagtatcagcaaagacccgaagtattcggacatccttgaggtactgcaaaaagtgtatctgaaactggaaaaacagaaatatgagttagatccaggaccattgatcaatcgtctggtaaactacctgtacttcaccgcctataccaacaagattcgtttcaccgaatatcaagaagagttaatccgcaatctgtctgagattggacgcacagcaggaatcaacggactgtaccgtgccgactatggagataaaagtcagttctaattgacgggcccatgagcctaggtcagtgcggccgctacggacatgtccgtagctagcgaaaaaaaaacccgcccctgacagggcgggtttttttttaatttaacccccctacgattaattaaactgaagctttccaccataatgcc

SEQ ID NO: 72 - DNA, 235 bp, artificial
  pMPES multiple cloning site
gagaattcaaggaggaaacagctgtgagctcccacagtagttacagagggcccctgcagctatagcgaccctaggcctagaacatatagcggccgcccatcacactgtgagcacatgtggacacttcaccacgtatctgggctagcgaaaaaaaaacccgcccctgacagggcgggtttttttttaatttaacccccctacgattaattaaactgaagctttccaccataatgcc

SEQ ID NO: 73 - DNA, 14195 bp, artificial
  pMPES nucleotide sequence
ctacatatcctggtatttttttccgattatctataacttgacgtgcaacggaaatttgccgtttagccactttaccgctattaccatggctacaatcaattatcaggcgatggttaatgccctcatcatgcattaatttcactgccttagttatatcagacaaaccatagttaggttctctacctccacgtaatattagatgtccatgtagattgccatcggttaatagtgtagatatactatttgttaaagatgtcatatatacaatatgttgttcacgggctgcaataatagcgtcaatagctaagttaatatttccatcagtactgtttttaaatccaactggacaatgtaaaccagacgcaagttgacgatgcgtttgtgatttcagtggttctggcaccaatagcaccccaacatattaaatcagcaatatagggagtaagaaaaggatcgaggaattctgtagcggttgcgacacgcatcgttgtgattgaagacaaacactggcgagcatatcgtatccctttctcaacatcataacttccatttagatcaggatcgtgcattattcccttccagcctttacgggttcgaggtttctcaaaataagtgcgcattacaatgtacattttcgataagtacttattctgtaatacgaacaatcgcttcgcataatcaactgcggcctgaacgtcatggattgagcatggaccaacaatcacaagtaagcgaggatcttttcccagcaaaatattcgctataatatctcgttgcagagagatccatgttacggtttcttccgatactgcgatttctttatgaatctcgcccacagtgggtaaactgccaagcaacttcccgcggtagattaatttactagctgactgcatcttgtctcctaaaagcgctatttcagaccatcagttgcacacattagatgataatgataatcatttactgtataattggcaatatatggggattcatagtcatttctgagataatatacgttaggccttctctcccgtacggaaatcacttgttttccgtaaattacctgaatcttaatccctgagctgcggctagctgtggaactcacaaaagctgaccgggccgtggcacagcctctctgctggcagaacagcgtactgcagaagtcgtcgtactgccgctggtcccggcatcaggtctgcccgcagatggcacttctgccactgatctgccggtggagctgaacggcatgacatatcaggcctgccgtggggattttgtggtgcgcctcgacggcagcacctgtctgcagttatagaataaagaaggcggggaggtacgcagggagggcgatccgctggaagtggcgtagtggctgtaggcctgtaatgatgcaggaatagaagttcgtgtacaggttaatgaaagcatcaccccgtaaaaacatgccggcacggtttacacatcattccatggtggcagccggttaactttcctgcgccaccagtggaaagaggccatgagcagtccgaacaaaaaaccactcgtgatacttataataatggcctcacctgctaccattcctgacggcccccagtacataaaccacattgcacatccccaggaaataccccataagccccccatcagcagcgtaacctgccagaacggcataaagggcaatggcggaagccggatacccagccgccagagaatacgcagcagaggaggagcataattactccgccacatctttttgctgtccatcagggcaattgcccgggctttttttgctcaaaagtcacaggagcacctccgttccatgatgtaaacgtgacacaacttattgttattgattattattatcataccgtgattatgttgtcatgcacctgtacactgataaaagaaagggagaacaggaggctggctgaacaaccaatccgttcgtccagttcaatggcggctatcaggttctgctccgcaatgtactcacgaatgcgcttcctgtcctggctggccagcatggtccagaatatctccatcatttaccggcgactttcctgctcatttcatcccgccatgcagcagcggaagattcgaccatgtcatggggaagcatatcgccgcgctcgagttgctgacgtcctgcctcaacctggtcacggaaccaggtattatgctgccgttccacggtctggcgcataaaatcccggattaactgagagccattacggtccatgctttttgcagggccataaaagcatccttcagttcagcgtcaattcttaaactcatattaacctgtgccatttatcaccccgtcattaccacgtatatacacagtatataacgatcagatatcgtcactaggatatgccgcgccagcggcatggaaggcggcactccgctgtttcatatgataccgccgaagcccgatgtaagccgctacagtcgtccgaaagtcaccagcctcctcccccctgccgtcatccgtgcatcagatatgcactgagtatgcctgcccttccctagagaatcctgccaggcttgccacactgatatatcttgactttatgtaaacgatatgacactttaacatgataatgattaccattctcttttaatatacagagaaactaggaaatagatgaatgagttatgttactttaatattctctgacaataacctaaatcagttagattattgtcatttaataaataatgacattctttcatcataaataaaaagactattgtttataatattgttctcagcattatatgattatttatcctgataactctcctatgttgtatgtttatatgattttccttgaaacatataatgcaaattttcgatttattttccatcattaatccagataaacaacaaactaatagtatgcaaggagacattatttgtttcgccaggatgctttagaaaacagaaaaatgaagtggcagggacgggcaatattacttcccggaataccactgtggttaatcatgctgggaagcattgtgtttattacggcatttctgatgttcattattgttggtacctatagccgccgtgttaatgtcagtggtgaggtcacacctggccaagagctgtcaatatatattcaggtgtacagggatttgttgtcaggcagtttgttcatgaagggcagttgataaaaaaaggggatcctgtttatctgattgacatcagtaaaagtacacgcaatggtattgtcactgataatcatcgccgggatatagaaaaccagctggttcgtgtggacaacattatttcccgtctggaagaaagtaaaaaaataacgctagataccctggaaaaacaacgtctgcaatacacagatgcgttccgtcgctcatcagacattatacagcgtgcagaggaagggataaaaataatgaaaaataatatggagaattacagatactatcagtcaaaaggactgattaataaagatcaattaactaaccaagttgcattatattatcaacaacaaaacaaccttctcagtctgagcggacaaaatgaacaaaatgccctgcagataaccactctggagagtcagattcagactcaggcagcagattttgataatcgtatctatcagatggaactgcaacgactcgaattgcagaaagaactggttaacactgatgtggaaggcgaaatcattatccgggcgttgtctgacgggaaagttgactccctgagtgtcactgtagggcaaatggtcaataccggagacagccttctgcaggttattcctgagaacattgaaaactattatcttattctctgggtcccgaatgatgctgttccttatatttcggctggtgacaaagtgaatattcgttatgaagccttcccctcagaaaaatttgggcagttctctgctacggttaaaactatatccaggactcctgcgtcaacacaggaaatgttgacctataagggagcacctcaaaatacgccgggtgcctctgttccctggtataaagtcattgcgacgcctgaaaagcagataatcaggtatgacgaaaaatacctccctctggaaaatggaatgaaagccgaaagtacactatttctggaaaaaaggcgtatttaccagtggatgctttctcctttctatgacatgaaacacagtgcaacaggaccgatcaatgactaacaggaatttcagacaaattataaatctgcttgatttgcgctggcaacgtcgtgttccggttattcatcagacagagaccgctgaatgtggactggcctgcctagcaatgatatgcggtcattttggtaagaatattgacctgatatatcttcgccggaagtttaatctctctgcccgtggagcaacccttgcaggaatcaatggaatagcggagcaactggggatggccacccgggctctttcactggagttggatgaacttcgagtcctcaaaacgccgtgtattctccactgggatttcagtcacttcgtcgttctggtcagcgtaaagcgtaaccgttatgtactgcatgatccggccaggggcataagatatatcagccgggaggaaatgagccgatattttacaggcgttgcacttgaggtctggcccggaagtgaattccagtcggaaaccctgcagacccgcataagtcttcgttcactgattaacagtatttacggtattaaaagaacgctggcgaaaattttctgtctgtcagttgtaattgaagcaatcaatctgctaatgccggtggggacacagctggttatggatcatgctattcctgcgggggacagagggctactgacgctaatttctgctgctcttatgttttttatattactcaaagctgcaacgagtacgctgcgcgcatggtcttcactggttatgagcacgctcatcaatgtacagtggcagtcggggctgttcgatcatcttctcagactaccgctggcgttttttgaacgccgaaaattaggtgatatccagtcacgttttgactcccttgacacattgagggccacatttaccaccagtgtgatcgggttcataatggacagcattatggttgtcggtgtttgtgtgatgatgctgttatacggaggatatctcacctggatagttctctgctttaccacaatttacatttttattcgactggtgacatacggcaattaccgacagatatcagaagaatgtcttgtcagggaggcccgtgccgcctcctattttatggaaacattatatggtattgccacggtaaaaatccaggggatggtcggaattcggggggcacactggcttaatatgaaaatagatgcgataaattcgggtattaagctaaccaggatggatttgctcttcggaggaataaatacctttgttaccgcctgtgatcagattgtaattttatggctgggagcaggccttgtgatcgataatcagatgacaataggaatgtttgtagcgtttagttcttttcgtgggcagttttcggaaagagttgcctctctgaccagttttcttcttcagctaagaataatgagtctgcacaatgagcgcattgcagatattgcattacatgaaaaggaggaaaagaaacctgaaattgaaatcgttgctgatatggggccaatatccctggaaaccaatggtttaagctatcgttatgacagtcagtcagcaccgatattcagtgctctgagtttatctgtagctccgggggaaagtgtggctataactggtgcttccggtgcgggaaaaaccacattaatgaaagtactatgtggactatttgaacctgatagcgggagggtactgataaatggtatagatatacgccaaattggaataaataattatcaccggatgatagcctgtgttatgcaggatgaccggctattttcaggctcaattcgtgaaaatatctgtggttttgcagaggaaatggatgaagagtggatggtagaatgtgccagagcaagtcatattcatgatgttataatgaatatgccaatgggatatgaaacattaataggtgaacttggggaaggtctttctggcggtcaaaaacagcgtatatttattgcacgagccttataccggaaaccaggaatattatttatggatgaggcaaccagtgctcttgattcagagagtgaacatttcgtgaatgttgccataaaaaacatgaatatcaccagggtaattattgcacacagagaaacaacgttgagaactgttgatagagttatttctatttaaaccatagaggaattacaagcgtatgaggaatatttcttcctgttataattcctcgttatgctcagatatctgttggaggtggaatggaagatagacaatccaccaagaagaaatatcattctgtgtggattgtccaataactgttctttcttatattaaataatactatttataaacaaacatcactaagattatttggactccaattacacaatcttcccgcagcatagttccatgcttctgaaggtatcccttcgggtttttgcttaattgttccccctaaaccggatggagacattgcaggattaggtttgtgagtggatgcatagtcatatattgcacctccagccacacccccagcagctgctccaattcctcctgcaacaaattgcccggatagtgttcctatagccatcgcaatatcacgccctgaagcaccaccagaaacagaatctaattcatttagagtcagagttctcatatgatctcctttttatcttatcggatattgaataataattatcaccaacaaagtaacatattgcagacattaatgcagagaagcaaaatgtatgcatggataaaaagtcctttcctctaaaaacacaatcatatatagctaatgcaatatatattgcggtggcatttattataaatgcaaataacaactctaattttgttctttttctatccattactttttatcccattactttctatcccattaccacacaaacactaacgataatgattatcgttaacatagtcaagagtgaagggtaggaggccctcaaccccctataaggggtccgcttggaaaacggatttccccacgtcaagagaattgatttgaacgagtggcttcgctacaaacagctcccgtttgtgtgtgaaaaacccctctcaacagatctcaactctgcacgatatctacaggagattgctccaaaaggcagtatcgtgtcttttaactaccgatataaccccgtcatcgacatgataatgcgtctgagaaatgagaagaaattaggtgaactgcatttcttctcagcagaatttaataaaaattctgcactgacccgccatcacctgacctggcgagactccgcacaacagagcaaaagcagcggtgctctgggcgatctatcctgtcatctgcttgatttattttgttttataggggaaagcccggtagtggttcatgggatcaaaacggtaaaggggacacgtgggtacaaaatcagatggtcaggttgaagtggatgataacggctatgtaatgggcagttcagaaaagggagcttatttcagagttcatgccagtaaatcggaaacagatcataatttgggactgcatatacagcttgtatttgaaaatggtgaaatcagatattcaacccaccatgaaaatcgcctgctcctgattctatttaatgatacgaatacggaaacaattggcttcgacgctctgaaacgcttgcctgatccaccacgggagcttccattctggtctgattcgtttattcatcttcatgacgactggtgtgctcttattaaatatggtcactcgtctcctaagctggcggacctgtcatccggccttcatatacaggaaataattgaggcattctgatatggataccattcttttaaccgggctgtttgctgcatttttcacaacgtttgcgttcgctccccagagcataaaaactatcaggacacgcaataccgaaggtatctccgttgttatgtatatcatgttcctgacaggtgtaatctcatggatagcgtatggcattatgcgatctgattttgccgtactgatcgccaatattgtcacactgtttctggctgcaccagtgcttgtaataacacttatcaaccgcaggaaaaaacacgtatgaatattgcaatcatcggcgcgggtcctgccggcatcatttctgcccgtaatgcgattaaagcaggtcactccgtggttctgtttgagaagaatacccgaattggggggatctggaacccctggagtggtggcgcttaccgtaatgcctgtatgcagaactctcgttatacatttcattatactggctttccccctggcgatatcgatgaatttccaggagtggaacaggtattcagatatctttcggccgtggccggagaggatgccctccgtgagtcgatccgactgaatactgaagttgtttcactcagaaaagacgccggacaccgggtgatccgctgcgcttctgaaggaaaagacacggaagacatttttgaccgggtcatcattgcaacaggtgaactctggcaaccccgccggccccccctgccaggtgaggaaaacttctccggaacgttgatcacgtcgagagattatcaggagccagaggcatttaaaggaaaaaatatcctcatcattggcggcggtgtcagcggtgcggatattgcctcagaccttgttccctttgccagaagcgtaagtctgtccgtcaaaaagatgggactttatctgccgagacaattcccgactggcccgaatgacatgatgcactcctatctgggcaggtgtctgctgagccaaatgaattatgaagattttatcggttatctcgacaccatgatgcctgactacatgcaggcctaccgagcatccagtctgttgcccgacatggcgaacaataacgcggtgcatgttaacgaaaaaatcataccgaatgtggccgccggtctgataaaagttaaaccccaggcagagcgtttcaccggagaaggcgctattaagtttcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgatactagatagatgacctcggggagcccgcctaatgagcgggctttttgcgcgccactctatcattgactctatcattgatagagtacttaacataagcacctgtaggatcgtacaggtttagcgaagaaaatggtttgttatagtcgaataaacctcgagttatctcgagtgagatattgttgacgcaccaaggaggaagcttctatgatgagccgtctggataaaagcaaagtgattaatagcgcactggaactgctgaatgaagttggtattgaaggtctgaccacccgtaaactggcccagaaactgggtgttgaacagccgaccctgtattggcatgtgaaaaataaacgtgcactgctggatgcactggccgttgaaattctggctcgccatcatgattatagcctgcctgcagcaggcgaaagctggcagagctttctgcgtaataatgccatgagctttcgtcgtgccctgctgcgttatcgtgatggtgcaaaagaacatctgggcacccgtccggatgaaaaacagtatgataccgttgaaacccagctgcgttttatgaccgaaaatggttttagcctgcgtgatggtctgtatgcaattagcgcagttagccattttaccctgggtgccgttctggaacagcaggaacataccgcagcactgaccgatcgtcctccggcaccggatgaaaatctgcctccgctgctgcgtgaagcactgatgattatggattctgatgatggtgaacaggcatttctgcatggtctggaaagcctgattcgtggttttgaagttcagctgaccgcactgctgcagattgttggtggtggtggtgcacgtacccagtatagcgaaagcatgggtgcccgtacacagtattctgaatctatgggtgctcgcacccagtattcagaaagtatgggtgcaagaacacagtatagcgagtctatgggagcgcgtactcagtatagtgaatcaatgggaggtggtatgccgagcctggttgataattaccgcaagattaatattgccaataataaaagcaacaacgatctgaccaaacgtgaaaaagaatgtctggcctgggcatgtgaaggtaaaagcagctgggatattagcaaaattctgggttgtagcgaacgtaccgtgacctttcatctgaccaatgcccagatgaaactgaataccaccaatcgttgtcagagcattagcaaagcaattctgaccggtgccattgattgtccgtatttcaaaaactgagaattcaaggaggaaacagctgtgagctcccacagtagttacagagggcccctgcagctatagcgaccctaggcctagaacatatagcggccgcccatcacactgtgagcacatgtggacacttcaccacgtatctgggctagcgaaaaaaaaacccgcccctgacagggcgggtttttttttaatttaacccccctacgattaattaaactgaagctttccaccataatgccag